(19) United States
(12) Patent Application Publication
(10) Pub. No.: US 2023/0312583 A1
(43) Pub. Date: Oct. 5, 2023

(54) 1H-IMIDAZO [4,5-H] QUINAZOLINE COMPOUND AS NOVEL SELECTIVE FLT3 INHIBITORS

(71) Applicant: SHENGKE PHARMACEUTICALS (JIANGSU) LTD., Suzhou, Jiangsu (CN)

(72) Inventors: Chengzhi Yu, San Diego, CA (US); Zuopeng Sun, Shenyang (CN); Bin Liu, San Diego, CA (US); Chenggang Zhang, Auckland (NZ)

(21) Appl. No.: 18/023,035

(22) PCT Filed: Aug. 27, 2021
(86) PCT No.: PCT/CN2021/114930

§ 371 Date: Feb. 24, 2023

(30) Foreign Application Priority Data
Aug. 27, 2020 (WO) PCT/CN2020/111656

(51) Int. Cl. C07D 487/04

(52) U.S. Cl. CPC C07D 487/04

(57) ABSTRACT

Provided is a 1H-imidazo [4,5-h] quinazoline compound of formula (I). The compound is a broad spectrum inhibitor having strong activity for FLT3 kinase, and is applicable in treating cell proliferative disorders.

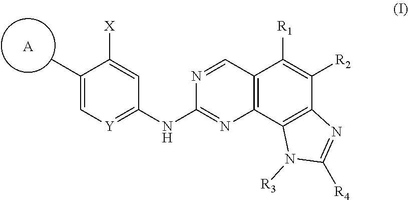

### TECHNICAL FIELD

The present disclosure relates to 1H-imidazo[4,5-h]quinazoline compounds having biological activities for inhibiting cell proliferation, modulating serine-threonine protein kinase activity, and modulating tyrosine kinase activity. More specifically, the present disclosure provides 1H-imidazo[4,5-h]quinazoline compounds as novel selective Flt3 kinase inhibitors, which are pan-inhibitors of Flt3 and its mutants, and are effective in the treatment of cell proliferative disorders related to Flt3 and its mutants.

### BACKGROUND OF THE INVENTION

Leukemia is a broad term for cancers of the blood cells. The type of leukemia depends on the type of blood cell that becomes cancer and whether it grows quickly or slowly. Leukemia occurs most often in adults older than 55, but it is also the most common cancer in children younger than 15. Acute myeloid leukemia (AML) is the most common form of acute leukemia in adults and the second most common leukemia in children. AML is characterized by the malignant transformation of a hematopoietic stem/progenitor cell (HSC). This occurs following the acquisition of somatic driver mutations that cooperate with accrued passenger mutations, or lesions that coincidentally occur following the acquisition of the driver mutations. The malignant precursor cells accumulate in the bone marrow and blood at the expense of healthy blood cells, leading to acute symptoms including anemia, bleeding and bruising, infections, and bone pain.

One kinase family of particular interest in AML is FLT3 (FMS-related tyrosine kinase 3). FLT3 is a membrane-spanning protein and composed of four domains; an extracellular ligand-binding domains consisting of five immunoglobin-like structures, a transmembrane (TM) domain, a juxtamembrane (JM) domain and a cytoplasmic C-Terminal tyrosine kinase (TK) domain. (Agnes F, et al. Gene 1994; 145:283-288; Scheijen B, et al. Oncogene 2002; 21:3314-3333). FLT3 is overexpressed at the levels in 70-100% of cases of AML, and in a high percentage of T-acute lymphocytic leukemia (ALL) cases (Griffin J D, et al. Haematol J. 2004; 5:188-190). It is also overexpressed in a smaller subset of chronic myeloid leukemia (CML) in blast crisis.

Evidence is rapidly accumulating that many types of leukemias and myeloproliferative syndromes have mutation in tyrosine kinases. There are two types of activating mutations in FLT3 described in patients with leukemia. These include a spectrum of internal tandem duplications (ITD) occurring within the auto-inhibitory juxtamembrane domain (Nakao M, et al. Leukemia 1996; 10:1911-1918; Thiede C, et al. Blood 2002; 99:4326-4335), and activation loop mutations that include Asp835Tyr (D835Y), Asp835Val (D835V), Asp835H is (D835H), Asp835Glu (D835E), Asp835Ala (D835A), Asp835Asn (D835N), Asp835 deletion and Ile836 deletion (Yamamoto Y, et al., Blood 2001:97:2434-2439; Abu-Duhier F M, et al. Br. J. Haematol. 2001; 113:983-988). Internal tandem duplication (ITD) mutations within the JM domain contribute to about 17-34% of FLT3 activating mutations in AML.

Due to the adverse significance and high frequency of FLT3, several FLT3 tyrosine kinase inhibitors have been developed. These inhibitors act via competitive inhibition with adenosine triphosphate on the TK domain, resulting in decreased autophosphorylation and its successive activation. First-generation FLT3 inhibitors, including tandutinib, sorafenib, midostaurin, lestaurtinib, SU11248, SU5614, and SU5416, are relatively nonspecific for FLT3 and usually inhibit other class III RTKs such as KIT and PDGFR. Second-generation FLT3 inhibitors, including quizartinib, crenolanib, ponatinib, pacritinib, and gilteritinib, are more potent and selective.

However, the therapeutic benefit of FLT3 inhibition, particularly as a monotherapy, frequently results in the development of treatment resistance and disease relapse. In addition, off-target inhibition may be associated with increased toxicity and modest clinical benefit. Therefore, there is still unmet needs for developing novel potent and selective FLT3 inhibitors.

### SUMMARY OF THE INVENTION

The present disclosure provides 1H-imidazo[4,5-h]quinazoline compounds as selective inhibitors of the Flt3 tyrosine kinase, as well as the mutants thereof, such as ITD, D835Y and F691L. The compounds disclosed herein are effective in the treatment of cell proliferative disorders related to Flt3 and mutants thereof.

In one aspect, the present disclosure provides a compound of formula (I), or a pharmaceutically acceptable salt, enantiomer, diastereomer, racemate, or mixture thereof:

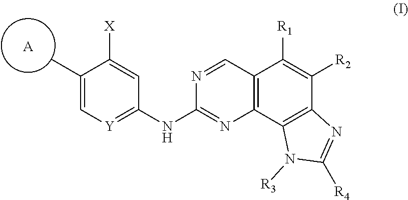

wherein:
Y is N, or CR₆;
   wherein R₆ is H, —ORₐ, —SRₐ, —NR_{b}R_{c}, —C(O)Rₐ, —C(O)ORₐ, —C(O)NR_{b}R_{c}, —O—C(O)Rₐ, —O—C(O)ORₐ, —O—C(O)NR_{b}R_{c}, —N(R_{b})—C(O)Rₐ, —N(R_{b})—C(O)ORₐ, or —N(R_{b})—C(O)NR_{b}R_{c}, C₁₋₆ alkyl, or C₁₋₆ haloalkyl;
X is —ORₐ, —SRₐ, —NR_{b}R_{c}, —C(O)Rₐ, —C(O)ORₐ, —C(O)NR_{b}R_{c}, —O—C(O)Rₐ, —O—C(O)ORₐ, —O—C(O)NR_{b}R_{c}, —N(R_{b})—C(O)Rₐ, —N(R_{b})—C(O)ORₐ, or —N(R_{b})—C(O)NR_{b}R_{c};
   wherein Rₐ is H, C₁₋₆ alkyl, C₁₋₆ haloalkyl, C₂₋₆ alkenyl, C₂₋₆ alkynyl, -L-C₃₋₇ cycloalkyl, -L-3- to 7-membered heterocyclyl, -L-C₆₋₁₀ aryl, or -L-5- to 10-membered heteroaryl;
   R_{b} is H, C₁₋₆ alkyl, C₁₋₆ haloalkyl, C₂₋₆ alkenyl, C₂₋₆ alkynyl, -L-C₃₋₇ cycloalkyl, -L-3- to 7-membered heterocyclyl, -L-C₆₋₁₀ aryl, or -L-5- to 10-membered heteroaryl;
   R_{c} is H, C₁₋₆ alkyl, C₁₋₆ haloalkyl, C₂₋₆ alkenyl, C₂₋₆ alkynyl, -L-C₃₋₇ cycloalkyl, -L-3- to 7-membered heterocyclyl, -L-C₆₋₁₀ aryl, or -L-5- to 10-membered heteroaryl;
   or R_{b}, R_{c} and N atom to which they are attached to form a 3- to 7-membered heterocyclyl, or 5- to 10-membered heteroaryl;
      wherein L is selected from a chemical bond, —C₁₋₆ alkylene-, —C₂₋₆ alkenylene-, or —C₂₋₆ alkynylene-;
ring A is -L′-3- to 11-membered heterocyclyl, which is optionally substituted with 1, 2, 3, 4, 5, 6, 7, or 8 R₅ groups;
   wherein L′ is selected from a chemical bond, —O—, —S—, —NH—, —O—CH₂—, —CH₂—O—, —NH—CH₂—, or —CH₂—NH—;
   R₅ is H, halo, oxo, —OR, —SR—, —NR′R″, C₁₋₆ alkyl, or C₁₋₆ haloalkyl; or, two of R₅s may link together to form a —C₁₋₄ alkylene-, —C₂₋₄ alkenylene- or —C₂₋₄ alkynylene-;
      R, R′ and R″ is each independently H, C₁₋₆ alkyl, C₁₋₆ haloalkyl, C₂₋₆ alkenyl, or C₂₋₆ alkynyl; or R′, R″, and N atom to which they are attached to form a 3- to 7-membered heterocyclyl, or 5- to 10-membered heteroaryl;
R₁ is H, halogen, —CN, —ORₐ, —SRₐ, —NR_{b}R_{c}, C₁₋₆ alkyl, or C₁₋₆ haloalkyl;
R₂ is H, halogen, —CN, —ORₐ, —SRₐ, —NR_{b}R_{c}, C₁₋₆ alkyl, or C₁₋₆ haloalkyl;
R₃ is C₁₋₆ alkyl, C₁₋₆ haloalkyl, C₃₋₇ cycloalkyl, 3- to 7-membered heterocyclyl, C₆₋₁₀ aryl, or 5- to 10-membered heteroaryl; and
R₄ is H, halogen, —CN, —ORₐ, —SRₐ, —NR_{b}R_{c}, C₁₋₆ alkyl, or C₁₋₆ haloalkyl.

In another aspect, the present disclosure provides a pharmaceutical composition comprising a compound disclosed herein, and optionally pharmaceutically acceptable excipients.

In another aspect, the present disclosure provides a pharmaceutical composition comprising a compound disclosed herein and pharmaceutically acceptable excipients, which further comprises other therapeutic agent(s).

In another aspect, the present disclosure provides a kit comprising a compound disclosed herein, other therapeutic agent(s), and pharmaceutically acceptable carriers, adjuvants or vehicles.

In another aspect, the present disclosure provides a use of a compound disclosed herein in the manufacture of a medicament for treating and/or preventing a Flt3 mediated disease.

In another aspect, the present disclosure provides a method of treating and/or preventing a Flt3 mediated disease in a subject, comprising administering to the subject a compound disclosed herein or a composition disclosed herein.

In another aspect, the present disclosure provides a compound disclosed herein or a composition disclosed herein, for use in treating and/or preventing a Flt3 mediated disease.

In another aspect, the Flt3 mediated disease is a proliferative disease, which is selected from a leukemia, myeloma, myeloproliferative disease, mylodysplastic syndrome, idiopathic hypereosinophilic syndrome (HES), bladder cancer, breast cancer, cervical cancer, CNS cancer, colon cancer, esophageal cancer, head and neck cancer, liver cancer, lung cancer, nasopharyngeal cancer, neuroendocrine cancer, ovarian cancer, pancreatic cancer, prostate cancer, renal cancer, salivary gland cancer, small cell lung cancer, skin cancer, stomach cancer, testicular cancer, thyroid cancer, uterine cancer, and hematologic malignancy.

Other objects and advantages of the present disclosure will be apparent to those skilled in the art from the specific embodiments, examples and claims disclosed herein.

### Definition

### Chemical Definitions

Definitions of specific functional groups and chemical terms are described in more detail below.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂ 6, C₂-5, C₂-4, C₂₋₃, C₃₋₆, C₃₋₅, C₃₋₄, C₄₋₆, C₄₋₅ and C₅₋₆ alkyl.

It should be understood that when described herein any of the moieties defined forth below may be substituted with a variety of substituents, and that the respective definitions are intended to include such substituted moieties within their scope as set out below. Unless otherwise stated, the term “substituted” is to be defined as set out below.

“C₁₋₆ alkyl” refers to a radical of a straight or branched, saturated, monovalent hydrocarbon group having from 1 to 6 carbon atoms. In some embodiments, C₁₋₄ alkyl is preferred. Typical C₁₋₆ alkyl includes methyl, ethyl, n-propyl, isopropyl, n-butyl, tert-butyl, iso-butyl, pentyl, n-hexyl, iso-hexyl, and the like. The term “C₁₋₆ alkyl” also includes heteroalkyl groups in which carbon atoms may be replaced by 1 to 3 atoms selected from O, S, N or substituted nitrogen atoms. The alkyl group can be substituted at any available point of attachment, for example, by from 1 to 5 substituents, 1 to 3 substituents, or 1 substituent.

“C₂₋₆ alkenyl” refers to a straight or branched hydrocarbon group having from 2 to 6 carbon atoms and at least one carbon-carbon double bonds, including but not limited to ethenyl, 3-buten-1-yl, 2-ethenylbutyl, 3-hexen-1-yl, and the like. In some embodiments, C₂₋₄ alkenyl is preferred. The term “C₂₋₆ alkenyl” also includes heteroalkenyl groups in which carbon atoms may be replaced by 1 to 3 atoms selected from O, S, N or substituted nitrogen atoms. The alkenyl group can be substituted at any available point of attachment, for example, by from 1 to 5 substituents, 1 to 3 substituents, or 1 substituent.

“C₂₋₆ alkynyl” refers to a straight or branched hydrocarbon group having from 2 to 6 carbon atoms, wherein at least one carbon-carbon triple bonds and optionally one or more unsaturated double bonds are contained. In some embodiments, C₂₋₄ alkynyl is preferred. Typical alkynyl includes ethynyl, propynyl, iso-propynyl, butynyl, iso-butynyl, pentynyl and hexynyl. The term “C₂₋₆ alkynyl” also includes heteroalkynyl groups in which carbon atoms may be replaced by 1 to 3 atoms selected from O, S, N or substituted nitrogen atoms. The alkynyl group can be substituted at any available point of attachment, for example, by from 1 to 5 substituents, 1 to 3 substituents, or 1 substituent.

“—C₁₋₆ alkylene-, —C₂₋₆ alkenylene- or —C₂₋₆ alkynylene-” refers to a divalent group of the “C₁₋₆ alkyl, C₂₋₆ alkenyl or C₂₋₆ alkynyl” as defined above.

“C₁₋₆ alkylene” refers to a C₁₋₆ alkyl group wherein another hydrogen is removed to provide a divalent radical of alkylene, and which may be substituted or unsubstituted alkylene. In some embodiments, C₁₋₄ alkylene is particularly preferred. Unsubstituted alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted alkylene groups, e.g., substituted with one or more alkyl (methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), and the like.

“C₂₋₆ alkenylene” refers to a C₂₋₆ alkenyl group wherein another hydrogen is removed to provide a divalent radical of alkenylene, and which may be substituted or unsubstituted alkenylene. In some embodiments, C₂₋₄ alkenylene is particularly preferred. Exemplary unsubstituted alkenylene groups include, but are not limited to, ethenylene (—CH═CH—) and propenylene (e.g., —CH═CHCH₂—, —CH₂—CH═CH—). Exemplary substituted alkenylene groups, e.g., substituted with one or more alkyl (methyl) groups, include but are not limited to, substituted ethenylene (—C(CH₃)═CH—, —CH═C(CH₃)—), substituted propenylene (e.g., —C(CH₃)═CHCH₂—, —CH═C(CH₃)CH₂—, —CH═CHCH(CH₃)—, —CH═CHC(CH₃)₂—, —CH(CH₃)—CH═CH—, —C(CH₃)₂—CH═CH—, —CH₂—C(CH₃)═CH—, —CH₂—CH═C(CH₃)—), and the like.

“C₂₋₆ alkynylene” refers to a C₂₋₆ alkynyl group wherein another hydrogen is removed to provide a divalent radical of alkynylene, and which may be substituted or unsubstituted alkynylene. In some embodiments, C₂₋₄ alkynylene is particularly preferred. Exemplary alkynylene groups include, but are not limited to, ethynylene (—C≡C—), substituted or unsubstituted propynylene (—C≡CCH₂—), and the like.

“Halo” or “halogen” means fluorine (F), chlorine (Cl), bromine (Br) and iodine (I).

“C₁₋₆ haloalkyl” means the above “C₁₋₆ alkyl” which is substituted with one or more halogen groups. Examples include mono-, di-, and poly-halogenated, including perhalogenated, alkyl. A monohalogen substituent in the group may be an iodine, bromine, chlorine or fluorine atom; dihalogen substituents and polyhalogen substituents may be two or more identical halogen atoms or a combination of different halogens. Examples of preferred haloalkyl groups include monofluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. The haloalkyl group can be substituted at any available point of attachment, for example, by 1 to 5 substituents, 1 to 3 substituents or 1 substituent.

“C₃₋₇ cycloalkyl” refers to a radical of a non-aromatic cyclic hydrocarbon group having from 3 to 7 ring carbon atoms and zero heteroatoms. In some embodiments, C₃₋₆ cycloalkyl is especially preferred, and C₅₋₆ cycloalkyl is more preferred. Cycloalkyl also includes ring systems wherein the cycloalkyl ring, as defined above, is fused with one or more aryl or heteroaryl groups wherein the point of attachment is on the cycloalkyl ring, and in such instances, the number of carbons continue to designate the number of carbons in the cycloalkyl ring system. Exemplary cycloalkyl groups include, but is not limited to, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), and the like.

“3- to 11-membered heterocyclyl” refers to a radical of a 3- to 11-membered non-aromatic ring system having ring carbon atoms and 1 to 5 ring heteroatoms, wherein each heteroatom is independently selected from nitrogen, oxygen, sulfur, boron, phosphorus, and silicon. In heterocyclyl groups that contain one or more nitrogen atoms, the point of attachment can be a carbon or nitrogen atom, as valency permits. In some embodiments, 3- to 9-membered heterocyclyl is preferred, which is a radical of a 3- to 9-membered non-aromatic ring system having ring carbon atoms and 1 to 5 ring heteroatoms; in some embodiments, 3- to 7-membered heterocyclyl is preferred, which is a radical of a 3- to 7-membered non-aromatic ring system having ring carbon atoms and 1 to 4 ring heteroatoms; in some embodiments, 3- to 6-membered heterocyclyl is preferred, which is a radical of a 3- to 6-membered non-aromatic ring system having ring carbon atoms and 1 to 3 ring heteroatoms; in some embodiments, 4- to 6-membered heterocyclyl is preferred, which is a radical of a 4- to 6-membered non-aromatic ring system having ring carbon atoms and 1 to 3 ring heteroatoms; in some embodiments, 5- to 6-membered heterocyclyl is more preferred, which is a radical of a 5- to 6-membered non-aromatic ring system having ring carbon atoms and 1 to 3 ring heteroatoms. Heterocyclyl also includes ring systems wherein the heterocyclyl ring, as defined above, is fused with one or more cycloalkyl groups, wherein the point of attachment is on the cycloalkyl ring; or wherein the heterocyclyl ring, as defined above, is fused with one or more aryl or heteroaryl groups, wherein the point of attachment is on the heterocyclyl ring; and in such instances, the number of ring members continue to designate the number of ring members in the heterocyclyl ring system. Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing three heteroatoms include, without limitation, triazinanyl. Exemplary 7-membered heterocyclyl groups containing one heteroatom include, without limitation, azepanyl, oxepanyl and thiepanyl. Exemplary 5-membered heterocyclyl groups fused to a C₆ aryl ring (also referred to herein as a 5, 6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to a C₆ aryl ring (also referred to herein as a 6,6-bicyclic heterocyclic ring) include, without limitation, tetrahydroquinolinyl, tetrahydroisoquinolinyl, and the like.

The 3- to 11-membered heterocyclyl also includes a spiroheterocyclic group, that is, a group in which two rings (e.g., a heterocyclyl and a carbocyclyl) share one carbon atom, wherein at least one ring is a heterocyclyl as defined above. More specifically, the spiroheterocyclyl is a spiro ring formed by two 4-membered rings, two 5-membered rings, two 6-membered rings, one 4-membered ring and one 5-membered ring, one 4-membered ring and one 6-membered ring, or one 5-membered ring and one 6-membered ring, wherein at least one ring is a 4- to 6-membered heterocyclyl as defined above, 4- to 6-membered heterocyclyl containing 1, 2 or 3 O, N or S heteroatoms is preferred, and 4- to 6-membered heterocyclyl containing 1 N heteroatom is more preferred. Specific spiroheterocyclyl groups include, but are not limited to:

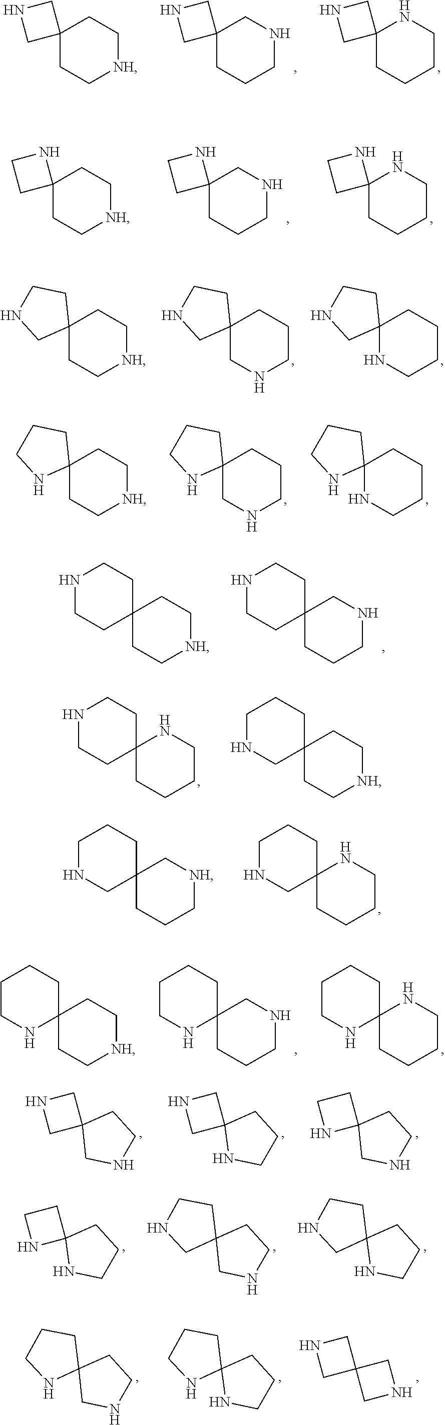

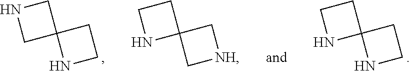 [SMILES: C1CC2(CNC2)N1 ; C1CC2(CNC2)N1 ; C1CC2(CCN2)N1]

Specific examples of preferred heterocyclyl groups include: pyrrolinyl, imidazolidinyl, pyrazolidinyl, tetrahydropyranyl, dihydropyranyl, dihydrofuryl, thiazolidinyl, dihydrothiazolyl, 2,3-dihydro-benzo[1,4]dioxol, indolinyl, isoindolinyl, dihydrobenzothiophene, dihydrobenzofuranyl, isodihydrobenzopyranyl, dihydrobenzopyranyl, 1,2-dihydroisoquinoline, 1,2,3,4-tetrahydroisoquinoline, 1,2,3,4-tetrahydroquinoline, 2,3,4,4a,9,9a-hexahydro-1H-3-azafluorene, 5,6,7-trihydro-1,2,4-triazolo[3,4-a]isoquinolyl, 3,4-dihydro-2H-benzo[1,4]oxazinyl, benzo[1,4]dioxol, 2,3-dihydro-1H-lk′-benzo[d]isothiazol-6-yl, 2,3-di-benzo[1,4]dioxinyl, dihydrobenzofuran, 2-oxoazirdin-1-yl, 2-oxoazetidin-1-yl, 2-oxopyrrolidin-1-yl, 2-oxopiperidin-1-yl, 2-oxoazepan-1-yl, 2-oxoazocan-1-yl, 2-oxoazonan-1-yl, 2-oxoazecan-1-yl, aziridine, azetidine, pyrrolidinyl, piperidine, azepane, azocane, azonane, azecane, piperidinyl, piperazinyl, morpholinyl, diazaspiro[3.3]heptane, diazaspiro[3.4]octane, diazaspiro[3.5]nonane, diazaspiro[4.4]nonane, diazaspiro[4.5]decane, and diazaspiro[5.5]undecane.

“C₆₋₁₀ aryl” refers to a radical of a monocyclic or polycyclic (e.g., bicyclic) 4n+2 aromatic ring system (e.g., having 6 or 10 π electrons shared in a cyclic array) having 6-10 ring carbon atoms and zero heteroatoms. In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). Aryl also includes ring systems wherein the aryl ring, as defined above, is fused with one or more cycloalkyl, or heterocyclyl groups wherein the point of attachment is on the aryl ring, and in such instances, the number of carbon atoms continue to designate the number of carbon atoms in the aryl ring system.

“5- to 10-membered heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms, wherein each heteroatom is independently selected from nitrogen, oxygen and sulfur. In heteroaryl groups that contain one or more nitrogen atoms, the point of attachment can be a carbon or nitrogen atom, as valency permits. Heteroaryl bicyclic ring systems can include one or more heteroatoms in one or both rings. Heteroaryl further includes ring systems wherein the heteroaryl ring, as defined above, is fused with one or more cycloalkyl, or heterocyclyl groups wherein the point of attachment is on the heteroaryl ring, and in such instances, the number of ring members continue to designate the number of ring members in the heteroaryl ring system. In some embodiments, 5- to 6-membered heteroaryl is especially preferred, which is a radical of a 5-6 membered monocyclic or bicyclic 4n+2 aromatic ring system having ring carbon atoms and 1-4 ring heteroatoms. Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Specific examples of preferred heteroaryl groups include: pyrrolyl, imidazolyl, pyrazolyl, 2-pyridyl, 3-pyridyl, 4-pyridyl, pyrimidinyl, pyrazinyl, pyridazinyl, triazolyl (4H-1,2,4-triazolyl, 1H-1,2,3-triazolyl, 2H-1,2,3-triazolyl), pyranyl, 2-furyl, 3-furan and etc., 2-thienyl, 3-thienyl, oxazolyl, isoxazolyl, oxazolyl (1,2,4-oxazolyl, 1,3,4-oxazolyl, 1,2,5-oxazolyl), thiazolyl, thiadiazolyl (1,2,4-thiadiazolyl, 1,3,4-thiadiazolyl, 1,2,5-thiadiazolyl).

“Oxo” represents ═O.

Alkyl, alkenyl, alkynyl, carbocyclyl, heterocyclyl, aryl, and heteroaryl groups, as defined herein, are optionally substituted groups. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), alkyl, haloalkyl, alkenyl, alkynyl, carbocyclyl, heterocyclyl, aryl, and heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, ═NNR^{bb}C(═O)R^{aa}, ═NNR^{bb}C(═O)ORₐ, ═NNR^{bb}S(═O)₂R^{aa}, ═NR^{bb}, or ═NOR^{cc};
each instance of R^{aa} is, independently, selected from alkyl, haloalkyl, alkenyl, alkynyl, carbocyclyl, heterocyclyl, aryl, and heteroaryl, or two R^{aa} groups are joined to form a heterocyclyl or heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{a}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{a})₂, —P(═O)₂N(R^{cc})₂, —P(═O)(NR^{c})₂, alkyl, haloalkyl, alkenyl, alkynyl, carbocyclyl, heterocyclyl, aryl, and heteroaryl, or two R^{bb} groups are joined to form a heterocyclyl or heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, alkyl, haloalkyl, alkenyl, alkynyl, carbocyclyl, heterocyclyl, aryl, and heteroaryl, or two R^{cc} groups are joined to form a heterocyclyl or heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, alkyl, haloalkyl, alkenyl, alkynyl, carbocyclyl, heterocyclyl, aryl, and heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from alkyl, haloalkyl, alkenyl, alkynyl, carbocyclyl, aryl, heterocyclyl, and heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, alkyl, haloalkyl, alkenyl, alkynyl, carbocyclyl, heterocyclyl, aryl and heteroaryl, or two R^{ff} groups are joined to form a heterocyclyl or heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)+X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ haloalkyl, C₂-C₆ alkenyl, C₂-C₆ alkynyl, C₃-C₇ carbocyclyl, C₆-C₁₀ aryl, C₃-C₇ heterocyclyl, C₅-C₁₀ heteroaryl; or two geminal R⁹⁹ substituents can be joined to form ═O or ═S; wherein X is a counterion.

Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, alkyl, haloalkyl, alkenyl, alkynyl, carbocyclyl, heterocyclyl, aryl, and heteroaryl, or two R^{cc} groups attached to a nitrogen atom are joined to form a heterocyclyl or heteroaryl ring, wherein each alkyl, alkenyl, alkynyl, carbocyclyl, heterocyclyl, aryl, and heteroaryl is independently substituted with 1, 2, 3, 4, or 5 R^{dd} groups, and wherein R^{a}, R^{bb}, R^{cc} and R^{dd} are as defined above.

### Other Definitions

The term “cancer” includes, but is not limited to, the following cancers: breast, ovary, cervix, prostate, testis, esophagus, stomach, skin, lung, bone, colon, pancreas, thyroid, biliary tract, buccal cavity and pharynx (mouth), lips, tongue, mouth, pharynx, small intestine, colorectal, large intestine, rectum, cancer of brain and central nervous system, glioblastoma, neuroblastoma, keratoacanthoma, epidermoid carcinoma, large cell carcinoma, adenocarcinoma, adenoma, follicular carcinoma, undifferentiated carcinoma, papillary carcinoma, seminoma, melanoma, sarcoma, bladder cancer, liver cancer, kidney cancer, bone marrow disorder, lymphatic disorder, Hodgkin's disease, hairy cell carcinoma and leukemia.

The term “pharmaceutically acceptable salt” as used herein denotes those carboxylate salts, and amino acid addition salts of the compounds disclosed herein, which are, within the scope of sound medical judgment, suitable for use in contact with the patient's tissue without undue toxicity, irritation, allergic response and the like, and are commensurate with a reasonable benefit/risk ratio, and are effective for their intended use, including, if possible, the zwitterionic form of the compounds disclosed herein.

The term “salt” denotes relatively non-toxic, inorganic and organic acid addition salts of the compounds disclosed herein. These salts can be prepared in situ during the final isolation and purification of the compound, or by isolating salts produced by separately reacting the purified compound in the free base form with a suitable organic or inorganic acid. As long as the compounds disclosed herein are basic compounds, they are capable of forming a plurality of different salts with various inorganic and organic acids. Although such salts must be pharmaceutically acceptable for administration to animals, it is often happened in practice that the pharmaceutically unacceptable salts of the basic compounds are first isolated from the reaction mixture, and then they are simply treated with an alkaline agent to convert to the free base compound, followed by the conversion of the free base to pharmaceutically acceptable acid addition salts. The acid addition salts of the basic compound are prepared by contacting the free base form with a sufficient amount of the desired acid in a conventional manner to form the salts. The free base can be regenerated by contacting the salt form with a base in a conventional manner and then isolating the free base. The free base forms differ somewhat in their physical properties from their respective salt forms, such as solubility in polar solvents, but for the purposes of the present invention, the salts are also equivalent to their respective free bases.

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

In general, the “effective amount” of a compound refers to an amount sufficient to elicit the desired biological response. As will be appreciated by those of ordinary skill in this art, the effective amount of a compound disclosed herein may vary depending on such factors as the desired biological endpoint, the pharmacokinetics of the compound, the disease being treated, the mode of administration, and the age, health, and condition of the subject. An effective amount encompasses therapeutically effective amount and prophylactically effective amount.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prophylaxis of the disease, disorder or condition. The term “prophylactically effective amount” can encompass an amount that improves overall prophylaxis or enhances the prophylactic efficacy of another prophylactic agent.

“Combination” and related terms mean the simultaneous or sequential administration of a compound disclosed herein and one or more other therapeutic agents. For example, the compound disclosed herein may be administered simultaneously or sequentially with other therapeutic agents in separate unit dosage forms, or together with one or more other therapeutic agents in a single unit dosage form.

### DETAILED DESCRIPTION OF THE EMBODIMENTS

As used herein, the term “compound disclosed herein” refers to the following compound of formula (I) or (II), or a pharmaceutically acceptable salt, enantiomer, diastereomer, racemate, or mixture thereof.

Compounds are generally described herein using standard nomenclature. For compounds having an asymmetric center, it should be understood, unless otherwise stated, that all optical isomers and mixtures thereof are included. Furthermore, all isomer compounds and carbon-carbon double bonds included in the present disclosure may occur in the form of Z and E unless otherwise specified. Compounds which exist in different tautomeric forms, one of which is not limited to any particular tautomer, but is intended to cover all tautomeric forms.

In one embodiment, the present disclosure refers to a compound of formula (I), or a pharmaceutically acceptable salt, enantiomer, diastereomer, racemate, or mixture thereof:

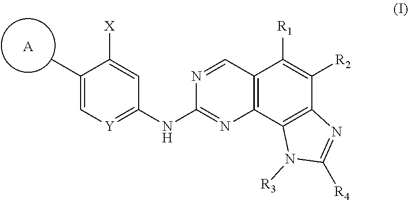

wherein:
Y is N, or CR₆;
   wherein R₆ is H, —ORₐ, —SRₐ, —NR_{b}R_{c}, —C(O)Rₐ, —C(O)ORₐ, —C(O)NR_{b}R_{c}, —O—C(O)Rₐ, —O—C(O)ORₐ, —O—C(O)NR_{b}R_{c}, —N(R_{b})—C(O)Rₐ, —N(R_{b})—C(O)ORₐ, or —N(R_{b})—C(O)NR_{b}R_{c}, C₁₋₆ alkyl, or C₁₋₆ haloalkyl;
X is —ORₐ, —SRₐ, —NR_{b}R_{c}, —C(O)Rₐ, —C(O)ORₐ, —C(O)NR_{b}R_{c}, —O—C(O)Rₐ, —O—C(O)ORₐ, —O—C(O)NR_{b}R_{c}, —N(R_{b})—C(O)Rₐ, —N(R_{b})—C(O)ORₐ, or —N(R_{b})—C(O)NR_{b}R_{c};
   wherein Rₐ is H, C₁₋₆ alkyl, C₁₋₆ haloalkyl, C₂₋₆ alkenyl, C₂₋₆ alkynyl, -L-C₃₋₇ cycloalkyl, -L-3- to 7-membered heterocyclyl, -L-C₆₋₁₀ aryl, or -L-5- to 10-membered heteroaryl;
   R_{b} is H, C₁₋₆ alkyl, C₁₋₆ haloalkyl, C₂₋₆ alkenyl, C₂₋₆ alkynyl, -L-C₃₋₇ cycloalkyl, -L-3- to 7-membered heterocyclyl, -L-C₆₋₁₀ aryl, or -L-5- to 10-membered heteroaryl;
   R_{c} is H, C₁₋₆ alkyl, C₁₋₆ haloalkyl, C₂₋₆ alkenyl, C₂₋₆ alkynyl, -L-C₃₋₇ cycloalkyl, -L-3- to 7-membered heterocyclyl, -L-C₆-10 aryl, or -L-5- to 10-membered heteroaryl;
   or R_{b}, R_{c} and N atom to which they are attached to form a 3- to 7-membered heterocyclyl, or 5- to 10-membered heteroaryl;
      wherein L is selected from a chemical bond, —C₁₋₆ alkylene-, —C₂₋₆ alkenylene-, or —C₂₋₆ alkynylene-;
ring A is -L′-3- to 11-membered heterocyclyl, which is optionally substituted with 1, 2, 3, 4, 5, 6, 7, or 8 R₅ groups;
   wherein L′ is selected from a chemical bond, —O—, —S—, —NH—, —O—CH₂—, —CH₂—O—, —NH—CH₂—, or —CH₂—NH—;
   R₅ is H, halo, oxo, —OR, —SR—, —NR′R″, C₁₋₆ alkyl, or C₁₋₆ haloalkyl; or, two of R₅s may link together to form a —C₁₋₄ alkylene-, —C₂₋₄ alkenylene- or —C₂₋₄ alkynylene-;
      R, R′ and R″ is each independently H, C₁₋₆ alkyl, C₁₋₆ haloalkyl, C₂₋₆ alkenyl, or C₂₋₆ alkynyl; or R′, R″, and N atom to which they are attached to form a 3- to 7-membered heterocyclyl, or 5- to 10-membered heteroaryl;
R₁ is H, halogen, —CN, —ORₐ, —SRₐ, —NR_{b}R_{c}, C₁₋₆ alkyl, or C₁₋₆ haloalkyl;
R₂ is H, halogen, —CN, —ORₐ, —SRₐ, —NR_{b}R_{c}, C₁₋₆ alkyl, or C₁₋₆ haloalkyl;
R₃ is C₁₋₆ alkyl, C₁₋₆ haloalkyl, C₃₋₇ cycloalkyl, 3- to 7-membered heterocyclyl, C₆₋₁₀ aryl, or 5- to 10-membered heteroaryl; and
R₄ is H, halogen, —CN, —ORₐ, —SRₐ, —NR_{b}R_{c}, C₁₋₆ alkyl, or C₁₋₆ haloalkyl.

### Y

In a specific embodiment, Y is N; in another specific embodiment, Y is CR₆; in another specific embodiment, Y is CH.

### X

In a specific embodiment, X is —ORₐ; in another specific embodiment, X is —SRₐ; in another specific embodiment, X is —NR_{b}R_{c}; in another specific embodiment, X is —C(O)Rₐ; in another specific embodiment, X is —C(O)ORₐ; in another specific embodiment, X is —C(O)NR_{b}R_{c}; in another specific embodiment, X is —O—C(O)Rₐ; in another specific embodiment, X is —O—C(O)ORₐ; in another specific embodiment, X is —O—C(O)NR_{b}R_{c}; in another specific embodiment, X is —N(R_{b})—C(O)Rₐ; in another specific embodiment, X is —N(R_{b})—C(O)ORₐ; in another specific embodiment, X is —N(R_{b})—C(O)NR_{b}R_{c}.

In a more specific embodiment of X, Rₐ is H; in another more specific embodiment of X, Rₐ is C₁₋₆ alkyl; in another more specific embodiment of X, Rₐ is C₁₋₆ haloalkyl; in another more specific embodiment of X, Rₐ is C₂₋₆ alkenyl; in another more specific embodiment of X, Rₐ is C₂₋₆ alkynyl; in another more specific embodiment of X, Rₐ is -L-C₃₇ cycloalkyl; in another more specific embodiment of X, Rₐ is -L-3- to 7-membered heterocyclyl; in another more specific embodiment of X, Rₐ is -L-C₆₋₁₀ aryl; in another more specific embodiment of X, Rₐ is -L-5- to 10-membered heteroaryl.

In a more specific embodiment of X, R_{b} is H; in another more specific embodiment of X, R_{b} is C₁₋₆ alkyl; in another more specific embodiment of X, R_{b} is C₁₋₆ haloalkyl; in another more specific embodiment of X, R_{b} is C₂₋₆ alkenyl; in another more specific embodiment of X, R_{b} is C₂₋₆ alkynyl; in another more specific embodiment of X, R_{b} is -L-C₃₋₇ cycloalkyl; in another more specific embodiment of X, R_{b} is -L-3- to 7-membered heterocyclyl; in another more specific embodiment of X, R_{b} is -L-C₆-10 aryl; in another more specific embodiment of X, R_{b} is -L-5- to 10-membered heteroaryl.

In a more specific embodiment of X, R_{c} is H; in another more specific embodiment of X, R_{c} is C₁₋₆ alkyl; in another more specific embodiment of X, R_{c} is C₁₋₆ haloalkyl; in another more specific embodiment of X, R_{c} is C₂₋₆ alkenyl; in another more specific embodiment of X, R_{c} is C₂₋₆ alkynyl; in another more specific embodiment of X, R_{c} is -L-C₃₋₇ cycloalkyl; in another more specific embodiment of X, R_{c} is -L-3- to 7-membered heterocyclyl; in another more specific embodiment of X, R_{c} is -L-C₆₋₁₀ aryl; in another more specific embodiment of X, R_{c} is -L-5- to 10-membered heteroaryl.

In a more specific embodiment of X, R_{b}, R_{c} and N atom to which they are attached to form a 3- to 7-membered heterocyclyl; in another more specific embodiment of X, R_{b}, R_{c} and N atom to which they are attached to form a 5- to 10-membered heteroaryl.

In a still more specific embodiment of Rₐ, R_{b} or R_{c}, L is a chemical bond; in another still more specific embodiment of Rₐ, R_{b} or R_{c}, L is —C₁₋₆ alkylene-; in another still more specific embodiment of Rₐ, R_{b} or R_{c}, L is —C₂₋₆ alkenylene-; in another still more specific embodiment of Rₐ, R_{b} or R_{c}, L is —C₂₋₆ alkynylene-.

### Ring A

In a specific embodiment, ring A is -L′-3- to 11-membered heterocyclyl; in another specific embodiment, ring A is 3- to 11-membered heterocyclyl; in another specific embodiment, ring A is -L′-3- to 7-membered heterocyclyl; in another specific embodiment, ring A is 3- to 7-membered heterocyclyl; in another specific embodiment, ring A is -L′-4- to 6-membered heterocyclyl; in another specific embodiment, ring A is 4- to 6-membered heterocyclyl; in another specific embodiment, ring A is 6-membered heterocyclyl.

In a more specific embodiment, ring A is

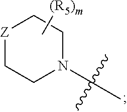

wherein Z is O, S, or NRs; R₅ is H, C₁₋₆ alkyl, or C₁₋₆ haloalkyl; or, two of R₅s may link together to form a —C₁₋₄ alkylene-, —C₂₋₄ alkenylene- or —C₂₋₄ alkynylene-; and m=1, 2, 3, 4, 5, 6, 7, or 8.

In a still more specific embodiment, ring A is

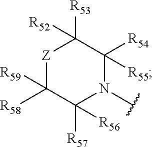

wherein Z: O, or NR₅₁; and R₅₁ to R₅₉ is H, or C₁₋₆ alkyl; or, two of R₅₁ to R₅₉ may link together to form a —C₁₋₄ alkylene-.

In a yet more specific embodiment, ring A is

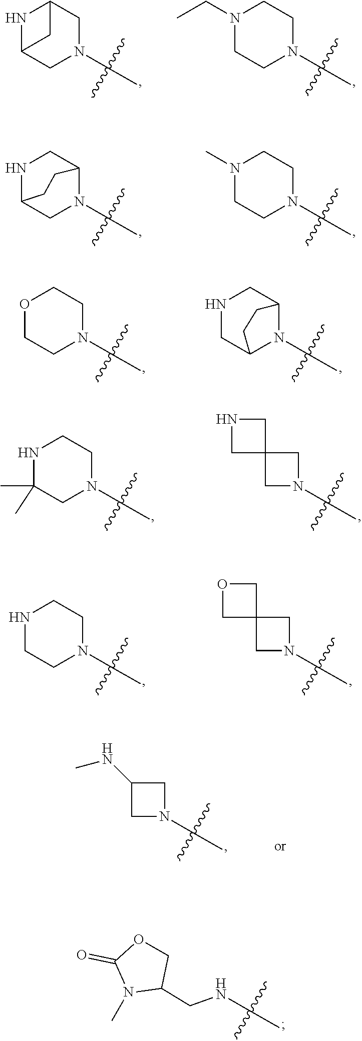

preferably, ring A is

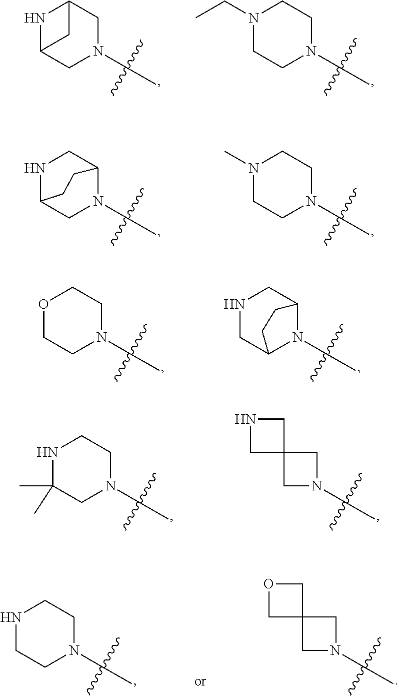

In a specific embodiment, ring A is unsubstituted; in another specific embodiment, ring A is substituted with 1 R₅ groups; in another specific embodiment, ring A is substituted with 2 R₅ groups; in another specific embodiment, ring A is substituted with 3 R₅ groups; in another specific embodiment, ring A is substituted with 4 R₅ groups; in another specific embodiment, ring A is substituted with 5 R₅ groups; in another specific embodiment, ring A is substituted with 6 R₅ groups; in another specific embodiment, ring A is substituted with 7 R₅ groups; in another specific embodiment, ring A is substituted with 8 R₅ groups.

In a specific embodiment of ring A, R₅ is H; in another specific embodiment of ring A, R₅ is halo; in another specific embodiment of ring A, R₅ is oxo; in another specific embodiment of ring A, R₅ is —OR; in another specific embodiment of ring A, R₅ is —SR—; in another specific embodiment of ring A, R₅ is —NR′R″; in another specific embodiment of ring A, R₅ is C₁₋₆ alkyl; in another specific embodiment of ring A, R₅ is C₁₋₆ haloalkyl; in another specific embodiment of ring A, two of R₅s may link together to form a —C₁₋₄ alkylene-; in another specific embodiment of ring A, two of R₅s may link together to form a —C₂₋₄ alkenylene-; in another specific embodiment of ring A, two of R₅s may link together to form a —C₂₋₄ alkynylene-.

### R₁

In a specific embodiment, R₁ is H; in another specific embodiment, R₁ is halogen; in another specific embodiment, R₁ is —CN; in another specific embodiment, R₁ is —ORₐ; in another specific embodiment, R₁ is —SRₐ; in another specific embodiment, R₁ is —NR_{b}R_{c}; in another specific embodiment, R₁ is C₁₋₆ alkyl; in another specific embodiment, R₁ is C₁₋₆ haloalkyl.

### R₂

In a specific embodiment, R₂ is H; in another specific embodiment, R₂ is halogen; in another specific embodiment, R₂ is —CN; in another specific embodiment, R₂ is —ORₐ; in another specific embodiment, R₂ is —SRₐ; in another specific embodiment, R₂ is —NR_{b}R_{c}; in another specific embodiment, R₂ is C₁₋₆ alkyl; in another specific embodiment, R₂ is C₁₋₆ haloalkyl.

### R₃

In a specific embodiment, R₃ is C₁₋₆ alkyl; in another specific embodiment, R₃ is C₁₋₆ haloalkyl; in another specific embodiment, R₃ is C₃₋₇ cycloalkyl; in another specific embodiment, R₃ is 3- to 7-membered heterocyclyl; in another specific embodiment, R₃ is C₆₋₁₀ aryl; in another specific embodiment, R₃ is 5- to 10-membered heteroaryl.

### R₄

In a specific embodiment, R₄ is H; in another specific embodiment, R₄ is halogen; in another specific embodiment, R₄ is —CN; in another specific embodiment, R₄ is —ORₐ; in another specific embodiment, R₄ is —SRₐ; in another specific embodiment, R₄ is —NR_{b}R_{c}; in another specific embodiment, R₄ is C₁₋₆ alkyl; in another specific embodiment, R₄ is C₁₋₆ haloalkyl.

Any technical solution in any one of the above specific embodiments, or any combination thereof, may be combined with any technical solution in other specific embodiments or any combination thereof. For example, any technical solution of Y or any combination thereof may be combined with any technical solution of X, ring A, R₁, R₂, R₃ and R₄ or any combination thereof. The present disclosure is intended to include all combination of such technical solutions, which are not exhaustively listed here to save space.

In a specific embodiment, the present disclosure refers to a compound of formula (I), or a pharmaceutically acceptable salt, enantiomer, diastereomer, racemate, or mixture thereof, wherein, Y is N.

In a specific embodiment, the present disclosure refers to a compound of formula (I), or a pharmaceutically acceptable salt, enantiomer, diastereomer, racemate, or mixture thereof, wherein,
X is —ORₐ, —SRₐ, —NR_{b}R_{c}, —C(O)Rₐ, —C(O)ORₐ, —C(O)NR_{b}R_{c}, —O—C(O)Rₐ, or —N(R_{b})—C(O)Rₐ;
preferably, X is —ORₐ, —SRₐ, or —NR_{b}R_{c};
preferably, X is —ORₐ;
preferably, X is —NR_{b}R_{c}.

In a specific embodiment, the present disclosure refers to a compound of formula (I), or a pharmaceutically acceptable salt, enantiomer, diastereomer, racemate, or mixture thereof, wherein,
Rₐ is H, C₁₋₆ alkyl, C₁₋₆ haloalkyl, C₂₋₆ alkenyl, C₂₋₆ alkynyl, -L-C₃₋₇ cycloalkyl, or -L-C₆₋₁₀ aryl; and
wherein L is selected from a chemical bond, or —C₁₋₆ alkylene-;
preferably,
Rₐ is C₁₋₆ alkyl, C₁₋₆ haloalkyl, C₂₋₆ alkenyl, C₂₋₆ alkynyl, or -L-C₃₋₇ cycloalkyl; and
wherein L is selected from a chemical bond, or —C₁₋₆ alkylene-.

In a specific embodiment, the present disclosure refers to a compound of formula (I), or a pharmaceutically acceptable salt, enantiomer, diastereomer, racemate, or mixture thereof, wherein,
R_{b} is H, C₁₋₆ alkyl, C₁₋₆ haloalkyl, C₂₋₆ alkenyl, C₂₋₆ alkynyl, -L-C₃₋₇ cycloalkyl, or -L-C₆₋₁₀ aryl;
R_{c} is H, C₁₋₆ alkyl, C₁₋₆ haloalkyl, C₂₋₆ alkenyl, C₂₋₆ alkynyl, -L-C₃₋₇ cycloalkyl, or -L-C₆₋₁₀ aryl;
or R_{b}, R_{c} and N atom to which they are attached to form a 3- to 7-membered heterocyclyl, or 5- to 10-membered heteroaryl; and
wherein L is selected from a chemical bond, or —C₁₋₆ alkylene-;
preferably,
R_{b} is H, C₁₋₆ alkyl, C₁₋₆ haloalkyl, C₂₋₆ alkenyl, C₂₋₆ alkynyl, or -L-C₃₋₇ cycloalkyl;
R_{c} is H, C₁₋₆ alkyl, C₁₋₆ haloalkyl, C₂₋₆ alkenyl, C₂₋₆ alkynyl, or -L-C₃₋₇ cycloalkyl;
or R_{b}, R_{c} and N atom to which they are attached to form a 3- to 7-membered heterocyclyl; and wherein L is selected from a chemical bond, or —C₁₋₆ alkylene-;
preferably,
R_{b} is H, C₁₋₆ alkyl, or C₁₋₆ haloalkyl;
R_{c} is H, C₁₋₆ alkyl, or C₁₋₆ haloalkyl;
or R_{b}, R_{c} and N atom to which they are attached to form a 4- to 6-membered heterocyclyl.

In a specific embodiment, the present disclosure refers to a compound of formula (I), or a pharmaceutically acceptable salt, enantiomer, diastereomer, racemate, or mixture thereof, wherein,
ring A is -L′-3- to 7-membered heterocyclyl, which is optionally substituted with 1, 2, 3, 4, 5, 6, 7, or 8 R₅ groups;
wherein L′ is selected from a chemical bond, —O—CH₂—, —CH₂—O—, —NH—CH₂—, or —CH₂—NH—;
R₅ is H, halo, oxo, —OR, —SR—, —NR′R″, C₁₋₆ alkyl, or C₁₋₆ haloalkyl; or, two of R₅s may link together to form a —C₁₋₄ alkylene-, —C₂₋₄ alkenylene- or —C₂₋₄ alkynylene-; and
R, R′ and R″ is each independently H, C₁₋₆ alkyl, or C₁₋₆ haloalkyl; or R′, R″, and N atom to which they are attached to form a 3- to 7-membered heterocyclyl, or 5- to 10-membered heteroaryl;
preferably,
ring A is -L′-4- to 6-membered heterocyclyl, which is optionally substituted with 1, 2, 3, 4, 5, 6, 7, or 8 R₅ groups;
wherein L′ is selected from a chemical bond, —O—CH₂—, —CH₂—O—, —NH—CH₂—, or —CH₂—NH—;
R₅ is H, oxo, —OR, —NR′R″, C₁₋₆ alkyl, or C₁₋₆ haloalkyl; or, two of R₅s may link together to form a —C₁₋₄ alkylene-; and
R, R′ and R″ is each independently H, C₁₋₆ alkyl, or C₁₋₆ haloalkyl; or R′, R″, and N atom to which they are attached to form a 4- to 6-membered heterocyclyl;
preferably,
ring A is -L′-4- to 6-membered heterocyclyl, which is optionally substituted with 1, 2, 3, 4, 5, 6, 7, or 8 R₅ groups;
wherein L′ is selected from a chemical bond, —O—CH₂—, —CH₂—O—, —NH—CH₂—, or —CH₂—NH—;
R₅ is H, oxo, —OR, —NR′R″, or C₁₋₆ alkyl; or, two of R₅s may link together to form a —C₁₋₄ alkylene-; and
R, R′ and R″ is each independently H, or C₁₋₆ alkyl; or R′, R″, and N atom to which they are attached to form a 4- to 6-membered heterocyclyl;
preferably,
ring A is

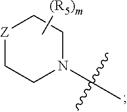

wherein Z is O, S, or NRS;
R₅ is H, C₁₋₆ alkyl, or C₁₋₆ haloalkyl; or, two of R₅s may link together to form a —C₁₋₄ alkylene-, —C₂₋₄ alkenylene- or —C₂₋₄ alkynylene-;
m=1, 2, 3, 4, 5, 6, 7, or 8;
preferably,
ring A is

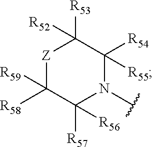

wherein Z is O, or NR₅₁;
R₅₁ to R₅₉ is H, or C₁₋₆ alkyl; or, two of R₅₁ to R₅₉ may link together to form a —C₁₋₄ alkylene-;
preferably
ring A is

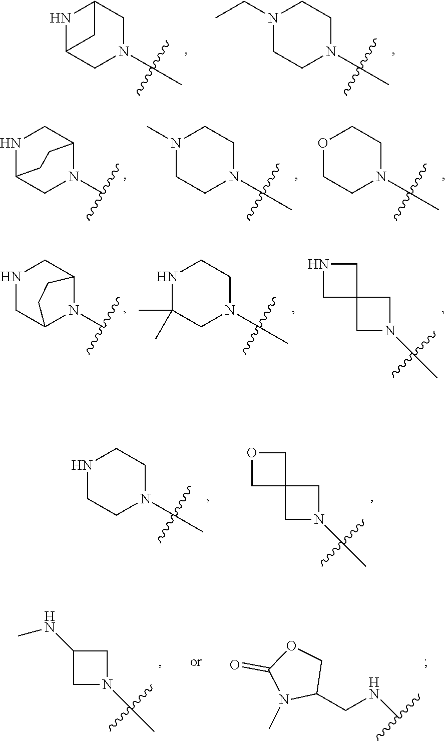

preferably,
ring A is

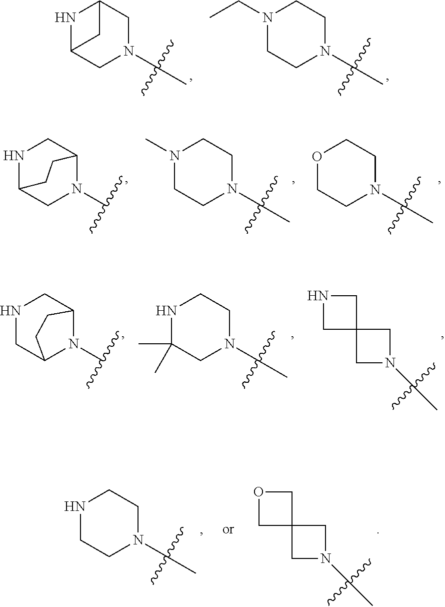

In a specific embodiment, the present disclosure refers to a compound of formula (I), or a pharmaceutically acceptable salt, enantiomer, diastereomer, racemate, or mixture thereof, wherein,
R₁ is H, C₁₋₆ alkyl, or C₁₋₆ haloalkyl;
preferably, R₁ is H.

In a specific embodiment, the present disclosure refers to a compound of formula (I), or a pharmaceutically acceptable salt, enantiomer, diastereomer, racemate, or mixture thereof, wherein,
R₂ is H, C₁₋₆ alkyl, or C₁₋₆ haloalkyl;
preferably, R₂ is H.

In a specific embodiment, the present disclosure refers to a compound of formula (I), or a pharmaceutically acceptable salt, enantiomer, diastereomer, racemate, or mixture thereof, wherein,
R₃ is C₁₋₆ alkyl, C₁₋₆ haloalkyl, C₃₋₇ cycloalkyl, or 3- to 7-membered heterocyclyl;
preferably, R₃ is C₁₋₆ alkyl, or C₁₋₆ haloalkyl;
preferably, R₃ is C₁₋₆ alkyl;
preferably, R₃ is Et or iPr.

In a specific embodiment, the present disclosure refers to a compound of formula (I), or a pharmaceutically acceptable salt, enantiomer, diastereomer, racemate, or mixture thereof, wherein,
R₄ is H, C₁₋₆ alkyl, or C₁₋₆ haloalkyl.
preferably, R₄ is H.

In a specific embodiment, the present disclosure refers to a compound of formula (I), or a pharmaceutically acceptable salt, enantiomer, diastereomer, racemate, or mixture thereof, wherein,
Y is N, or CR₆;
wherein R₆ is H, C₁₋₆ alkyl, or C₁₋₆ haloalkyl;
X is —ORₐ, —SRₐ, or —NR_{b}R_{c};
wherein Rₐ is H, C₁₋₆ alkyl, C₁₋₆ haloalkyl, C₂₋₆ alkenyl, C₂₋₆ alkynyl, -L-C₃₋₇ cycloalkyl, -L-3- to 7-membered heterocyclyl, -L-C₆₋₁₀ aryl, or -L-5- to 10-membered heteroaryl;
R_{b} is H, C₁₋₆ alkyl, or C₁₋₆ haloalkyl;
R_{c} is H, C₁₋₆ alkyl, or C₁₋₆ haloalkyl;
or R_{b}, R_{c} and N atom to which they are attached to form a 4- to 6-membered heterocyclyl, or 5- to 10-membered heteroaryl;
wherein L is selected from a chemical bond, —C₁₋₆ alkylene-, —C₂₋₆ alkenylene-, or —C₂₋₆ alkynylene-;
ring A is -L′-3- to 7-membered heterocyclyl, which is optionally substituted with 1, 2, 3, 4, 5, 6, 7, or 8 R₅ groups;
wherein L′ is selected from a chemical bond, —O—, —S—, —NH—, —O—CH₂—, —CH₂—O—, —NH—CH₂—, or —CH₂—NH—;
R₅ is H, halo, oxo, C₁₋₆ alkyl, C₁₋₆ haloalkyl, —OR, —SR—, or —NR′R″; or, two of R₅s may link together to form a —C₁₋₄ alkylene-, —C₂₋₄ alkenylene- or —C₂₋₄ alkynylene-;
R, R′ and R″ is each independently H, C₁₋₆ alkyl, or C₁₋₆ haloalkyl; or R′, R″, and N atom to which they are attached to form a 3- to 7-membered heterocyclyl, or 5- to 10-membered heteroaryl;
R₁ is H, C₁₋₆ alkyl, or C₁₋₆ haloalkyl;
R₂ is H, C₁₋₆ alkyl, or C₁₋₆ haloalkyl;
R₃ is C₁₋₆ alkyl, or C₁₋₆ haloalkyl; and
R₄ is H, C₁₋₆ alkyl, or C₁₋₆ haloalkyl.

In a specific embodiment, the present disclosure refers to a compound of formula (I), or a pharmaceutically acceptable salt, enantiomer, diastereomer, racemate, or mixture thereof, wherein,
Y is N, or CR₆;
wherein R₆ is H, or C₁₋₆ alkyl;
X is —ORₐ, or —NR_{b}R_{c};
wherein Rₐ is C₁₋₆ alkyl, C₁₋₆ haloalkyl, C₂₋₆ alkenyl, C₂₋₆ alkynyl, -L-C₃₋₇ cycloalkyl, or -L-C₆₋₁₀ aryl;
R_{b} is H, or C₁₋₆ alkyl;
R_{c} is H, or C₁₋₆ alkyl;
or R_{b}, R_{c} and N atom to which they are attached to form a 4- to 6-membered heterocyclyl;
wherein L is selected from a chemical bond, or —C₁₋₆ alkylene-;
ring A is -L′-4- to 6-membered heterocyclyl, which is optionally substituted with 1, 2, 3, 4, 5, 6, 7, or 8 R₅ groups;
wherein L′ is selected from a chemical bond, —O—CH₂—, —CH₂—O—, —NH—CH₂—, or —CH₂—NH—;
R₅ is H, oxo, C₁₋₆ alkyl, —OR, or —NR′R″; or, two of R₅s may link together to form a —C₁₋₄ alkylene-;
R, R′ and R″ is each independently H, or C₁₋₆ alkyl; or R′, R″, and N atom to which they are attached to form a 4- to 6-membered heterocyclyl, or 5- to 6-membered heteroaryl;
R₁ is H, or C₁₋₆ alkyl;
R₂ is H, or C₁₋₆ alkyl;
R₃ is C₁₋₆ alkyl; and
R₄ is H, or C₁₋₆ alkyl.

In a specific embodiment, the present disclosure refers to a compound of formula (I), or a pharmaceutically acceptable salt, enantiomer, diastereomer, racemate, or mixture thereof, wherein,
Y is N, or CH;
X is —ORₐ, or —NR_{b}R_{c};
wherein Rₐ is Me, Et, iPr,

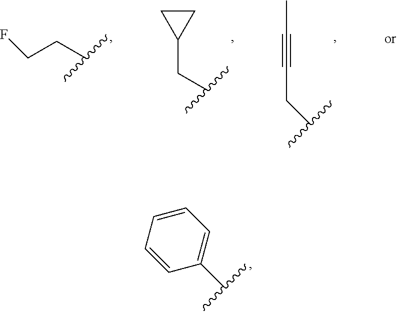

R_{b} is H, Me;
R_{c} is H, Me;
or R_{b}, R_{c} and N atom to which they are attached to form a

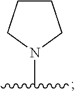 [SMILES: *N1CCCC1]

ring A is

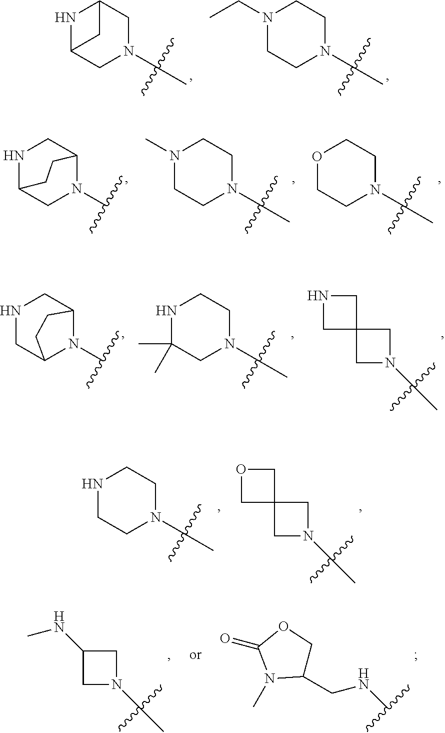

R₁ is H, or Me;
R₂ is H, or Me;
R₃ is Me, Et, or iPr; and
R₄ is H, or Me.

In a specific embodiment, the present disclosure refers to a compound of formula (I), or a pharmaceutically acceptable salt, enantiomer, diastereomer, racemate, or mixture thereof, which is a compound of formula (II):

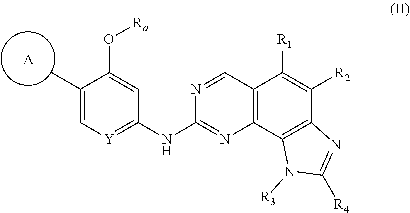

Y is N, or CH;
ring A is -L′-3- to 7-membered heterocyclyl, which is optionally substituted with 1, 2, 3, 4, 5, 6, 7, or 8 R₅ groups;
wherein L′ is selected from a chemical bond, —O—, —S—, —NH—, —O—CH₂—, —CH₂—O—, —NH—CH₂—, or —CH₂—NH—;
R₅ is H, halo, oxo, C₁₋₆ alkyl, C₁₋₆ haloalkyl, —OR, —SR—, or —NR′R″; or, two of R₅s may link together to form a —C₁₋₄ alkylene-, —C₂₋₄ alkenylene- or —C₂₋₄ alkynylene-;
R, R′ and R″ is each independently H, C₁₋₆ alkyl, or C₁₋₆ haloalkyl; or R′, R″, and N atom to which they are attached to form a 4- to 6-membered heterocyclyl, or 5- to 10-membered heteroaryl;
Rₐ is H, C₁₋₆ alkyl, C₁₋₆ haloalkyl, C₂₋₆ alkenyl, C₂₋₆ alkynyl, -L-C₃₋₇ cycloalkyl, -L-3- to 7-membered heterocyclyl, -L-C₆ 10 aryl, or -L-5- to 10-membered heteroaryl;
wherein L is selected from a chemical bond, —C₁₋₆ alkylene-, —C₂₋₆ alkenylene-, or —C₂₋₆ alkynylene-;
R₂ is H, C₁₋₆ alkyl, or C₁₋₆ haloalkyl;
R₃ is Et, or iPr; and
R₄ is H, C₁₋₆ alkyl, or C₁₋₆ haloalkyl.

In a specific embodiment, the present disclosure refers to a compound of formula (II), or a pharmaceutically acceptable salt, enantiomer, diastereomer, racemate, or mixture thereof, wherein,
Y is N, or CH;
ring A is -L′-4- to 6-membered heterocyclyl, which is optionally substituted with 1, 2, 3, 4, 5, 6, 7, or 8 R₅ groups;
L′ is selected from a chemical bond, —O—CH₂—, —CH₂—O—, —NH—CH₂—, or —CH₂—NH—;
R₅ is H, oxo, C₁₋₆ alkyl, —OR, or —NR′R″; or, two of R₅s may link together to form a —C₁₋₄ alkylene-;
R, R′ and R″ is each independently H, or C₁₋₆ alkyl; or R′, R″, and N atom to which they are attached to form a 4- to 6-membered heterocyclyl, or 5- to 6-membered heteroaryl;
Rₐ is C₁₋₆ alkyl, C₁₋₆ haloalkyl, C₂₋₆ alkenyl, C₂₋₆ alkynyl, -L-C₃₋₇ cycloalkyl, or -L-C₆₋₁₀ aryl;
wherein L is selected from a chemical bond, or —C₁₋₆ alkylene-;
R₂ is H, or C₁₋₆ alkyl;
R₃ is Et, or iPr; and
R₄ is H, or C₁₋₆ alkyl.

In a specific embodiment, the present disclosure refers to a compound of formula (II), or a pharmaceutically acceptable salt, enantiomer, diastereomer, racemate, or mixture thereof, wherein,
Y is N, or CH;
ring A is

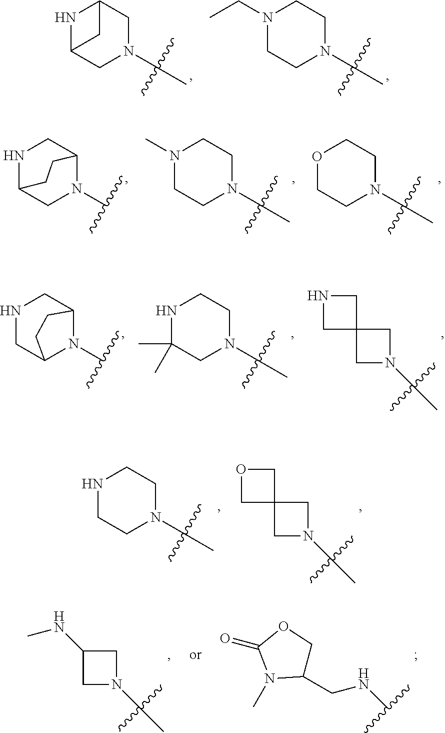

Rₐ is Me, Et, iPr,

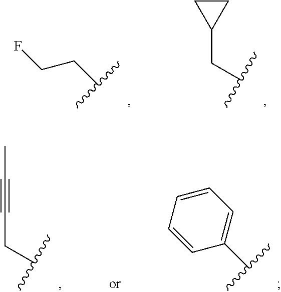

R₂ is H, or Me;
R₃ is Et, or iPr; and
R₄ is H, or Me.

In a specific embodiment, the present disclosure refers to a compound of formula (I), or a pharmaceutically acceptable salt, enantiomer, diastereomer, racemate, or mixture thereof, which is a compound of formula (II):

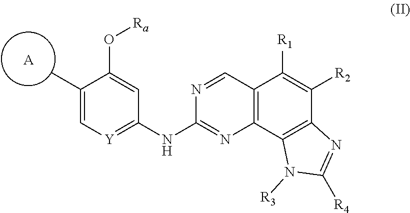

wherein:
Y is N, or CH;
ring A is

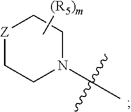

wherein Z is O, S, or NR₅;
R₅ is H, C₁₋₆ alkyl, or C₁₋₆ haloalkyl; or, two of R₅s may link together to form a —C₁₋₄ alkylene-, —C₂₋₄ alkenylene- or —C₂₋₄ alkynylene-;
m=1, 2, 3, 4, 5, 6, 7, or 8;
Rₐ is H, C₁₋₆ alkyl, C₁₋₆ haloalkyl, C₂₋₆ alkenyl, C₂₋₆ alkynyl, -L-C₃₋₇ cycloalkyl, or -L-3- to 7-membered heterocyclyl;
wherein L is selected from a chemical bond, —C₁₋₆ alkylene-, —C₂₋₆ alkenylene-, or —C₂₋₆ alkynylene-;
R₂ is H, C₁₋₆ alkyl, or C₁₋₆ haloalkyl;
R₃ is Et, or iPr; and
R₄ is H, C₁₋₆ alkyl, or C₁₋₆ haloalkyl.

In a specific embodiment, the present disclosure refers to a compound of formula (II), or a pharmaceutically acceptable salt, enantiomer, diastereomer, racemate, or mixture thereof, wherein,
Y is N, or CH;
ring A is

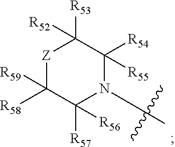

wherein Z is O, or NR₅₁;
R₅₁ to R₅₉ is each independently H, or C₁₋₆ alkyl; or, two of R₅₁ to R₅₉ may link together to form a —C₁₋₄ alkylene-;
Rₐ is C₁₋₆ alkyl, C₁₋₆ haloalkyl, C₂₋₆ alkenyl, C₂₋₆ alkynyl, or -L-C₃₋₇ cycloalkyl;
wherein L is selected from a chemical bond, or —C₁₋₆ alkylene-;
R₂ is H, or C₁₋₆ alkyl;
R₃ is Et, or iPr; and
R₄ is H, or C₁₋₆ alkyl.

In a specific embodiment, the present disclosure refers to a compound of formula (II), or a pharmaceutically acceptable salt, enantiomer, diastereomer, racemate, or mixture thereof, wherein,
Y is N, or CH;
ring A is

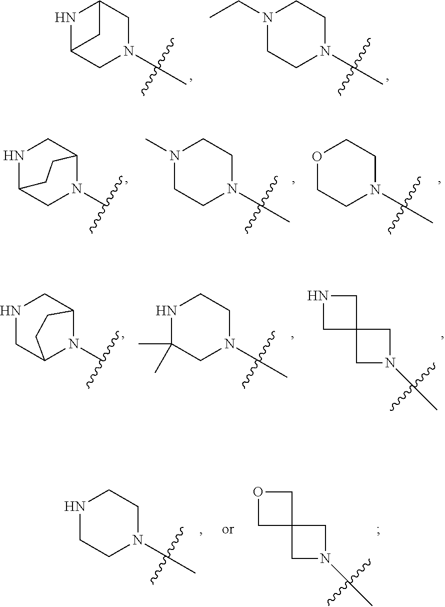

Rₐ is Me, Et, iPr,

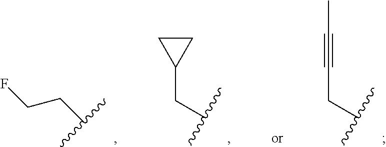

R₂ is H, or Me;
R₃ is Et, or iPr;
R₄ is H, or Me.

In a specific embodiment, the present disclosure refers to a compound as disclosed herein, or a pharmaceutically acceptable salt, enantiomer, diastereomer, racemate, or mixture thereof, wherein the said compound is selected from:

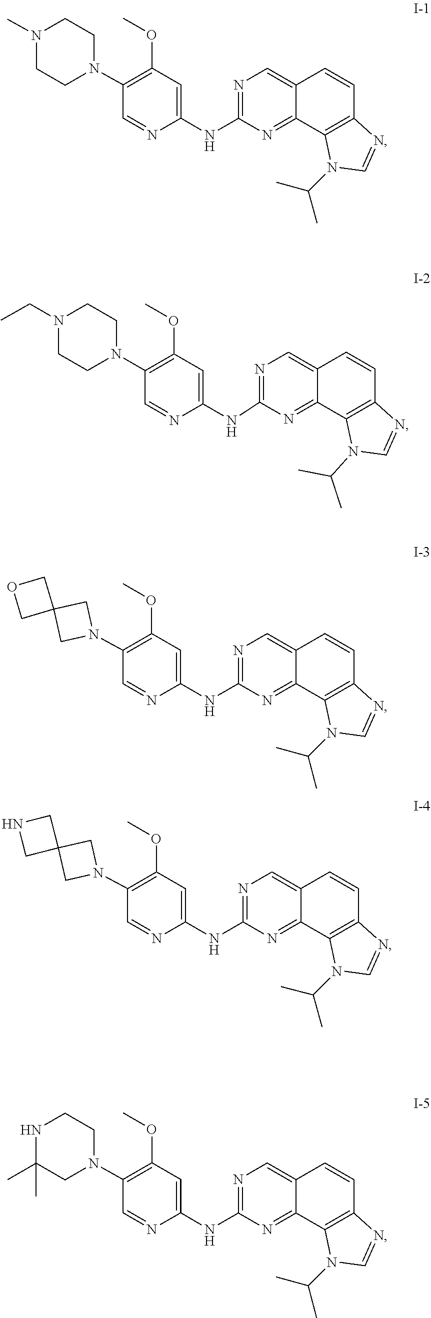 [SMILES: COc1cc(Nc2ncc3ccc4ncn(C(C)C)c4c3n2)ncc1N1CCN(C)CC1 ; CCN1CCN(c2cnc(Nc3ncc4ccc5ncn(C(C)C)c5c4n3)cc2OC)CC1 ; COc1cc(Nc2ncc3ccc4ncn(C(C)C)c4c3n2)ncc1N1CC2(COC2)C1 ; COc1cc(Nc2ncc3ccc4ncn(C(C)C)c4c3n2)ncc1N1CC2(CNC2)C1 ; COc1cc(Nc2ncc3ccc4ncn(C(C)C)c4c3n2)ncc1N1CCNC(C)(C)C1]

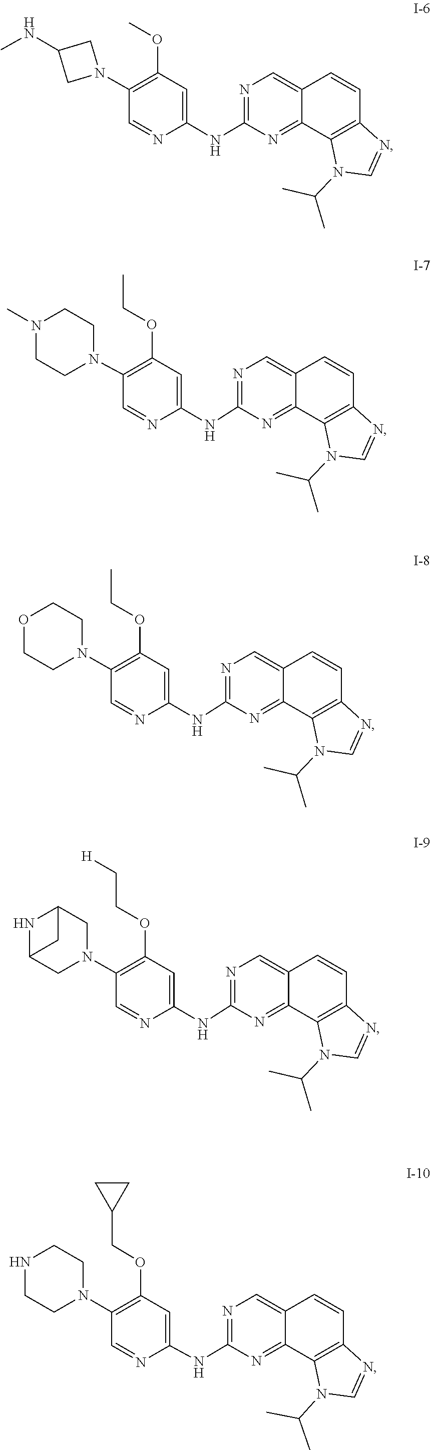 [SMILES: CNC1CN(c2cnc(Nc3ncc4ccc5ncn(C(C)C)c5c4n3)cc2OC)C1 ; CCOc1cc(Nc2ncc3ccc4ncn(C(C)C)c4c3n2)ncc1N1CCN(C)CC1 ; CCOc1cc(Nc2ncc3ccc4ncn(C(C)C)c4c3n2)ncc1N1CCOCC1 ; CCOc1cc(Nc2ncc3ccc4ncn(C(C)C)c4c3n2)ncc1N1CC2CC(C1)N2 ; CC(C)n1cnc2ccc3cnc(Nc4cc(OCC5CC5)c(N5CCNCC5)cn4)nc3c21]

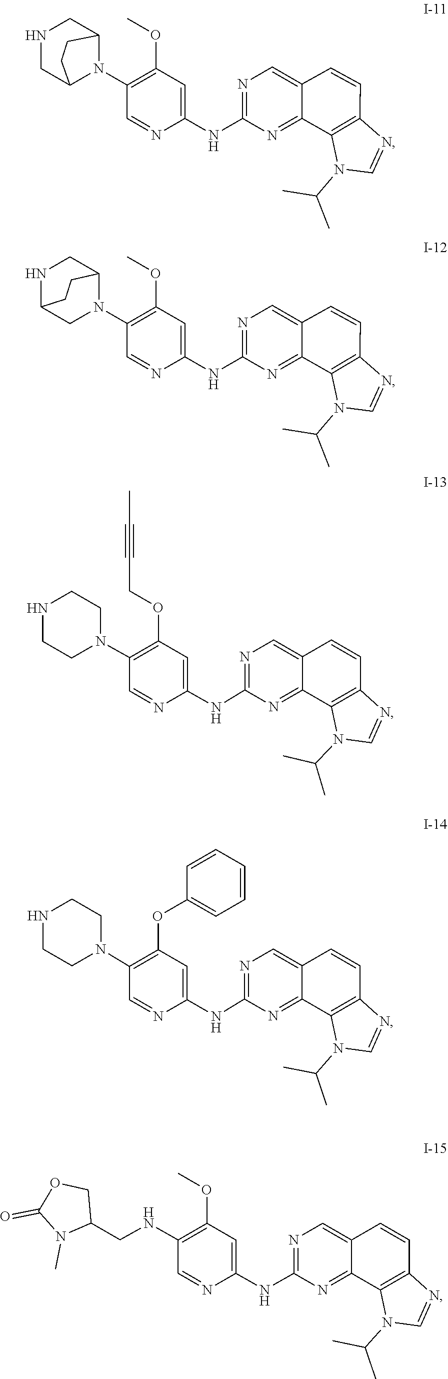

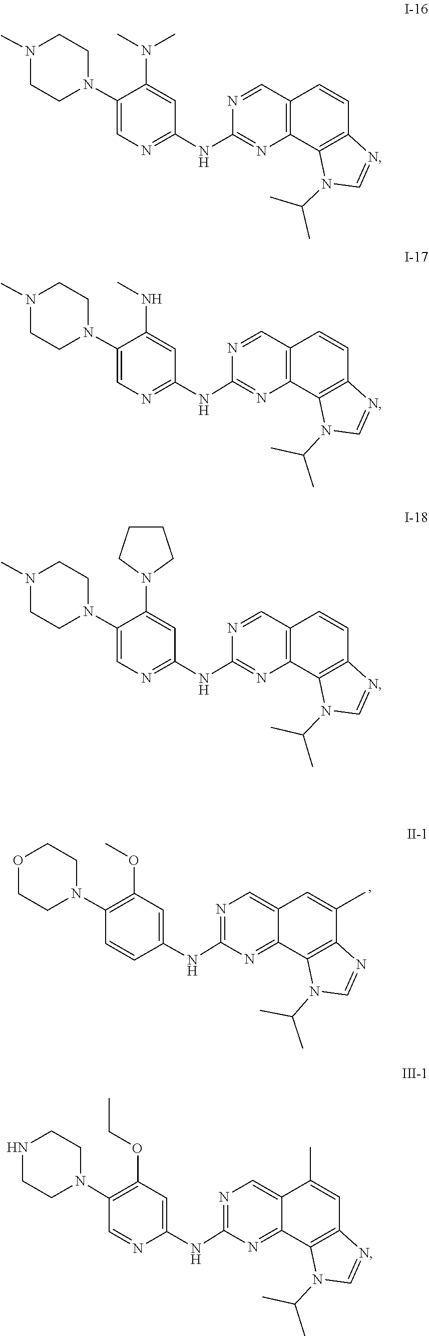 [SMILES: CC(C)n1cnc2ccc3cnc(Nc4cc(N(C)C)c(N5CCN(C)CC5)cn4)nc3c21 ; CNc1cc(Nc2ncc3ccc4ncn(C(C)C)c4c3n2)ncc1N1CCN(C)CC1 ; CC(C)n1cnc2ccc3cnc(Nc4cc(N5CCCC5)c(N5CCN(C)CC5)cn4)nc3c21 ; COc1cc(Nc2ncc3cc(C)c4ncn(C(C)C)c4c3n2)ccc1N1CCOCC1 ; CCOc1cc(Nc2ncc3c(C)cc4ncn(C(C)C)c4c3n2)ncc1N1CCNCC1]

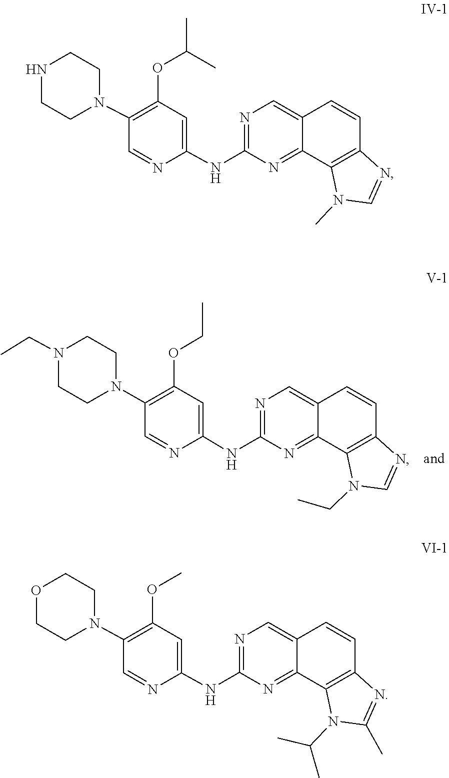 [SMILES: CC(C)Oc1cc(Nc2ncc3ccc4ncn(C)c4c3n2)ncc1N1CCNCC1 ; CCOc1cc(Nc2ncc3ccc4ncn(CC)c4c3n2)ncc1N1CCN(CC)CC1 ; COc1cc(Nc2ncc3ccc4nc(C)n(C(C)C)c4c3n2)ncc1N1CCOCC1]

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer (such as cis- and trans-isomer), or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses.

### Pharmaceutical Compositions, Formulations and Kits

In another aspect, the disclosure provides a pharmaceutical composition comprising a compound of the present disclosure (also referred to as the “active ingredient”) and a pharmaceutically acceptable excipient. In certain embodiments, the pharmaceutical composition comprises an effective amount of the compound of the present disclosure. In certain embodiments, the pharmaceutical composition comprises a therapeutically effective amount of the compound of the present disclosure. In certain embodiments, the pharmaceutical composition comprises a prophylactically effective amount of the compound of the present disclosure.

A pharmaceutically acceptable excipient for use in the present disclosure refers to a non-toxic carrier, adjuvant or vehicle which does not destroy the pharmacological activity of the compound formulated together. Pharmaceutically acceptable carriers, adjuvants, or vehicles that can be used in the compositions of the present disclosure include, but are not limited to, ion exchangers, alumina, aluminum stearate, lecithin, serum proteins (e.g., human serum albumin), buffer substances (such as phosphate), glycine, sorbic acid, potassium sorbate, a mixture of partial glycerides of saturated plant fatty acids, water, salt or electrolyte (such as protamine sulfate), disodium hydrogen phosphate, potassium hydrogen phosphate, sodium chloride, zinc salt, silica gel, magnesium trisilicate, polyvinyl pyrrolidone, cellulose-based materials, polyethylene glycol, sodium carboxymethyl cellulose, polyacrylate, wax, polyethylene-polyoxypropylene block polymers, polyethylene glycol and lanolin.

The present disclosure also includes kits (e.g., pharmaceutical packs). Kits provided may include a compound disclosed herein, other therapeutic agent(s), and a first and a second containers (eg, vials, ampoules, bottles, syringes, and/or dispersible packages or other suitable container) containing the compound disclosed herein or other therapeutic agent(s). In some embodiments, kits provided can also optionally include a third container containing a pharmaceutically acceptable excipient for diluting or suspending the compound disclosed herein and/or other therapeutic agent(s). In some embodiments, the compound disclosed herein provided in the first container and the other therapeutic agent(s) provided in the second container is combined to form a unit dosage form.

### Administration

The pharmaceutical composition provided by the present disclosure can be administered by a variety of routes including, but not limited to, oral administration, parenteral administration, inhalation administration, topical administration, rectal administration, nasal administration, oral administration, vaginal administration, administration by implant or other means of administration. For example, parenteral administration as used herein includes subcutaneous administration, intradermal administration, intravenous administration, intramuscular administration, intra-articular administration, intraarterial administration, intrasynovial administration, intrasternal administration, intracerebroventricular administration, intralesional administration, and intracranial injection or infusion techniques.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

When used to prevent the disorder disclosed herein, the compounds provided herein will be administered to a subject at risk for developing the condition, typically on the advice and under the supervision of a physician, at the dosage levels described above. Subjects at risk for developing a particular condition generally include those that have a family history of the condition, or those who have been identified by genetic testing or screening to be particularly susceptible to developing the condition.

The pharmaceutical compositions provided herein can also be administered chronically (“chronic administration”). Chronic administration refers to administration of a compound or pharmaceutical composition thereof over an extended period of time, e.g., for example, over 3 months, 6 months, 1 year, 2 years, 3 years, 5 years, etc, or may be continued indefinitely, for example, for the rest of the subject's life. In certain embodiments, the chronic administration is intended to provide a constant level of the compound in the blood, e.g., within the therapeutic window over the extended period of time.

The pharmaceutical compostions of the present disclosure may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or excipients and processing aids helpful for forming the desired dosing form.

With oral dosing, one to five and especially two to four and typically three oral doses per day are representative regimens. Using these dosing patterns, each dose provides from about 0.01 to about 20 mg/kg of the compound provided herein, with preferred doses each providing from about 0.1 to about 10 mg/kg, and especially about 1 to about 5 mg/kg.

Transdermal doses are generally selected to provide similar or lower blood levels than are achieved using injection doses, generally in an amount ranging from about 0.01 to about 20% by weight, preferably from about 0.1 to about 20% by weight, preferably from about 0.1 to about 10% by weight, and more preferably from about 0.5 to about 15% by weight.

Injection dose levels range from about 0.1 mg/kg/hour to at least 10 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 2 g/day for a 40 to 80 kg human patient.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable excipients known in the art. As before, the active compound in such compositions is typically a minor component, often being from about 0.05 to 10% by weight with the remainder being the injectable excipient and the like.

Transdermal compositions are typically formulated as a topical ointment or cream containing the active ingredient(s). When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or Formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The compounds of the present disclosure can also be administered in sustained release forms or from sustained release drug delivery systems. A description of representative sustained release materials can be found in Remington's Pharmaceutical Sciences.

The present disclosure also relates to the pharmaceutically acceptable formulations of a compound of the present disclosure. In one embodiment, the formulation comprises water. In another embodiment, the formulation comprises a cyclodextrin derivative. The most common cyclodextrins are α-, β- and γ-cyclodextrins consisting of 6, 7 and 8 α-1,4-linked glucose units, respectively, optionally comprising one or more substituents on the linked sugar moieties, which include, but are not limited to, methylated, hydroxyalkylated, acylated, and sulfoalkylether substitution. In certain embodiments, the cyclodextrin is a sulfoalkyl ether β-cyclodextrin, e.g., for example, sulfobutyl ether β-cyclodextrin, also known as Captisol. See, e.g., U.S. Pat. No. 5,376,645. In certain embodiments, the formulation comprises hexapropyl-β-cyclodextrin (e.g., 10-50% in water).

### Treatment

The present disclosure provides methods for treating the following disorders or conditions in mammals, including humans: cell proliferative disorders such as cancer, vascular smooth muscle hyperplasia associated with atherosclerosis, postoperative vascular stenosis, restenosis, and endometriosis; infection, including viral infections such as DNA viruses e.g. herpes, and RNA viruses e.g. HIV, and fungal infections; autoimmune diseases such as psoriasis, inflammation, e.g. rheumatoid arthritis, lupus, type I diabetes, diabetic nephropathy, multiple sclerosis and glomerulonephritis; organ transplant rejection, including host versus graft disease, the method comprises administering to the mammal a therapeutically effective amount of a compound disclosed herein or a composition thereof.

The present disclosure further provides compounds disclosed herein useful in the treatment of abnormal cell proliferation, such as cancer. The disclosure further provides a method of treating abnormal cell proliferation, such as cancer selected from the following: breast, ovary, cervix, prostate, testis, esophagus, stomach, skin, lung, bone, colon, pancreas, thyroid, biliary tract, buccal cavity and pharynx (mouth), lips, tongue, mouth, pharynx, small intestine, colorectal, large intestine, rectum, cancer of brain and central nervous system, glioblastoma, neuroblastoma, keratoacanthoma, epidermoid carcinoma, large cell carcinoma, adenocarcinoma, adenoma, follicular carcinoma, undifferentiated carcinoma, papillary carcinoma, seminoma, melanoma, sarcoma, bladder cancer, liver cancer, kidney cancer, bone marrow disorder, lymphatic disorder, Hodgkin's disease, hairy cell carcinoma and leukemia, comprising administering to the patient in need of such treatment a therapeutically effective amount of a compound disclosed herein or a composition thereof.

Further, the present disclosure relates to a method of treating a subject having a disease caused by proliferation of vascular smooth muscle cells. The compounds disclosed herein effectively inhibit the proliferation and migration of vascular smooth muscle cells. The method comprises administering to a subject in need of treatment a compound disclosed herein or a composition thereof in an amount sufficient to inhibit vascular smooth muscle proliferation and/or migration.

The present disclosure further provides a method of treating a subject suffering from gout, comprising administering to the subject in need of treatment a compound disclosed herein or a composition thereof in an amount sufficient to treat the condition.

The present disclosure further provides a method of treating a subject having a renal disease, such as a polycystic kidney disease, comprising administering to the subject in need of treatment an amount of a compound disclosed herein or a composition thereof in an amount sufficient to treat the condition.

Due to their inhibitory activity against Flt3 and other kinases, the compounds disclosed herein are also useful research tools for studying the mechanism of action of these kinases in vitro and in vivo.

The compounds disclosed herein are useful in the treatment of cancer (e.g., leukemia and cancers of lung, breast, prostate and skin, such as melanoma) and other proliferative diseases including, but not limited to, psoriasis, HSV, HIV, restenosis, and atherosclerosis. To treat cancer with a compound disclosed herein, a therapeutically effective amount of a pharmaceutically acceptable composition comprising at least one compound disclosed herein is administered to a patient in need of such treatment, for example, who has cancer or another proliferative disorder.

An effective amount of a compound disclosed herein will generally be administered in a single or multiple doses at an average daily dose of from 0.01 mg to 50 mg of compound per kilogram of patient body weight, preferably from 0.1 mg to 25 mg of compound per kilogram of patient body weight. In general, the compounds disclosed herein may be administered to a patient in need of such treatment in a daily dosage range of from about 1 mg to about 3500 mg per patient, preferably from 10 mg to 1000 mg. For example, the daily dose per patient can be 10, 20, 30, 40, 50, 60, 70, 80, 90, 100, 150, 200, 250, 300, 350, 400, 500, 600, 700, 800, 900 or 1000 mg. It can be administered one or more times daily, weekly (or several days apart) or on an intermittent schedule. For example, the compound can be administered one or more times per day on a weekly basis (e.g., every Monday), continually or for several weeks, such as 4-10 weeks. Alternatively, the administration may be continued for several days (e.g., 2-10 days), followed by a few days (e.g., 1-30 days) without administration of the compound, and the cycle may be repeated indefinitely or repeated for a given number of times, such as 4-10. Cycles. For example, the compounds disclosed herein may be administered daily for 5 days, then intermittently for 9 days, then administered daily for 5 days, then intermittent for 9 days, and so on, and the cycle is repeated indefinitely or repeated 4-10 times.

### EXAMPLES

The following examples are provided to provide those skilled in the art with a complete disclosure and description of how to implement, prepare and evaluate the methods and compounds claimed herein, and are intended to be illustrative only and not limiting the scope of the invention.

The preparation protocol of the compound disclosed herein is shown in following Schemes.

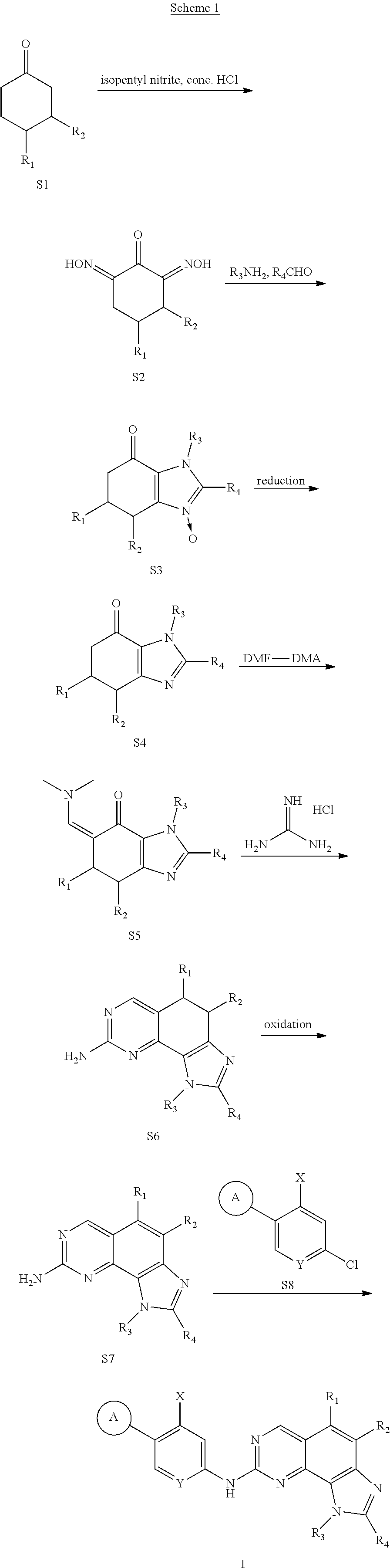

In step 1, S1 is reacted with conc. HCl and isopentyl nitrite to afford S2. In step 2, a mixture of S2 and R₄CHO is treated with R₃NH₂ to afford S3. In step 3, S3 is reduced to get S4. In step 4, S4 is reacted with DMF-DMA to give S5. In step 5, S5 is treated with guanidine hydrochloride to afford S6. In step 6, S6 is oxidized to give S7. In the last step 7, S7 is coupled with S8 to afford the title compound of formula (I).

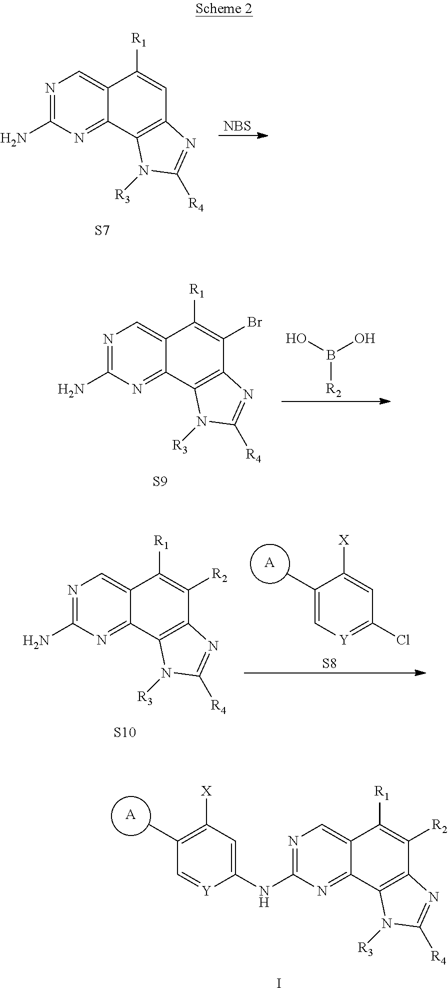

The compound of formula (I) can also be prepared using scheme 2, using S7 wherein R₂ is H as starting material, which is subjected to bromation following by coupling with a boronic acid.

### Example 1: Preparation of N-(5-(4-ethylpiperazin-1-yl)-4-methoxypyridin-2-yl)-1-isopropyl-1H-imidazo[4,5-h]quinazolin-8-amine (I-2)

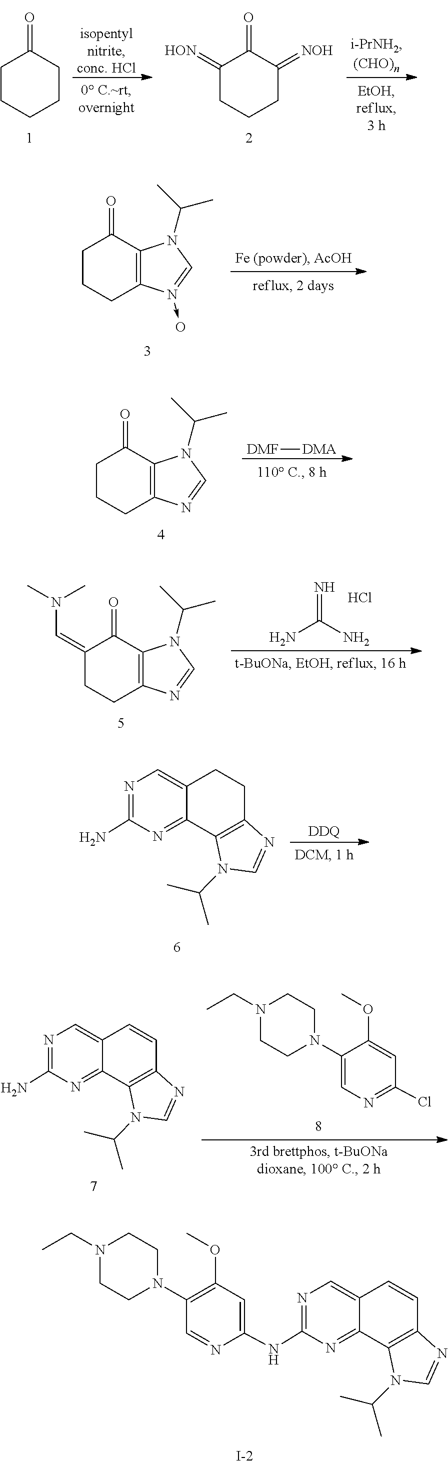

Step 1

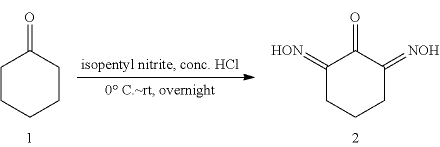

To 1 (450 g, 4.6 mol, 1.00 eq) was added conc. HCl (38 mL, 0.46 mol, 0.1 eq) and the mixture was cooled to 0° C. with ice water. Isopentyl nitrite (1076 g, 9.2 mol, 2.00 eq) was added at this temperature dropwise. Then the reaction mixture was allowed to warm to RT and stirred overnight. The solid was filtered and the filter cake was washed with petroleum ether (150 mL×2), dried in vacuum to afford the title compound (300 g, 95% purity) as a pale yellow solid.

Steps 2 and 3

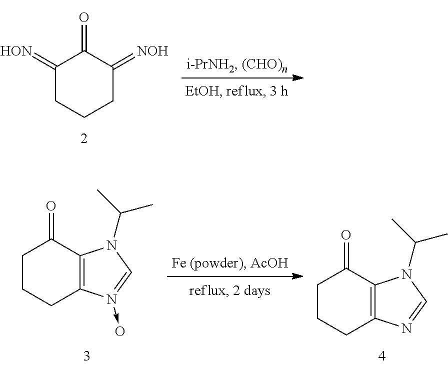

To a solution of 2 (300 g, 1.9 mol, 1.00 eq) and polyoxymethylene (43 g, 2.09 mmol, 1.10 eq) in EtOH (2.3 L) was added isopropyl amine (123 g, 2.09 mol, 1.10 eq) dropwise. The mixture was stirred at room temperature for 2 hours and then refluxed for another 3 hours. The mixture was concentrated to afford the crude compound 3.

To a solution of crude compound 3 in AcOH (2.1 L) was added Fe (powder) (620 g, 9.5 mol, 5 eq). The mixture was refluxed for 48 hours. After cooled to RT, the mixture was diluted with EtOAc (1000 mL) and filtered. The filtrate was concentrated. The residue was diluted with water (2000 mL) and basified with 2M NaOH (aq.) until pH ˜9. The mixture was then extracted with EtOAc (1000 mL×3). The organic layers were combined, dried over Na₂SO₄ and concentrated. The residue was purified by silica gel column chromatography eluted with PE/EtOAc (1/1) to afford the compound 4 (248 g, 96% purity) as brown oil.

Step 4

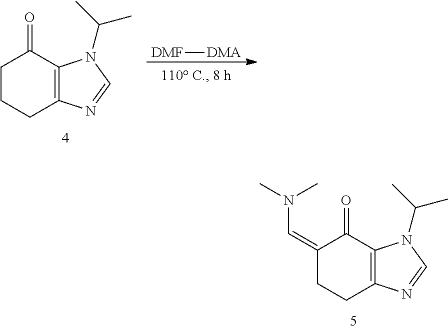

A solution of 4 (248 g, 1.4 mol, 1.00 eq) in DMF (700 mL) and DMF-DMA (700 mL) was heated at 110° C. for 8 h. The solvent was removed in vacuum to give 306 g of crude product 5 which was used in the next step directly without further purification.

Step 5

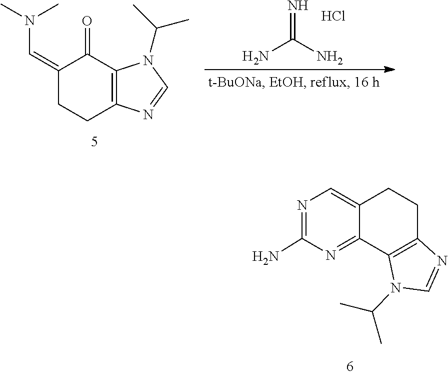

To a solution of 5 (306 g crude) in EtOH (2.5 L) was added guanidine hydrochloride (237 g) and t-BuONa (480 g). The mixture was stirred for 16 hours at 80° C. The solvent was removed in vacuo and the residue was purified by silica column chromatography eluted with PE/EtOAc (1/1) to afford the compound 6 (125 g, 95% purity) as a yellow solid.

Step 6

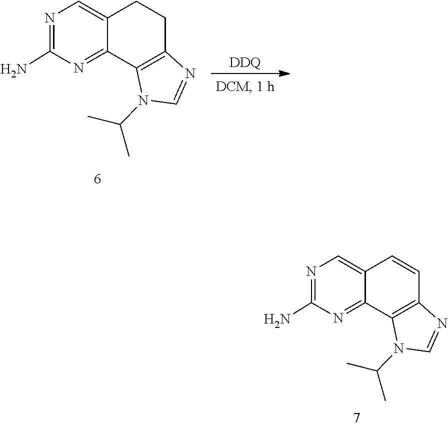

To a solution of 6 (22.9 g, 100 mmol, 1.00 eq) in DCM (800 mL) was added DDQ (27.2 g, 120 mmol, 1.20 eq). The mixture was stirred for 1 h at room temperature. The mixture solution was concentrated in vacuum. The residue was purified by silica gel column chromatography eluted with PE/EtOAc (1/1) to afford the compound 7 (11.3 g, 94% purity) as a yellow solid.

Step 7

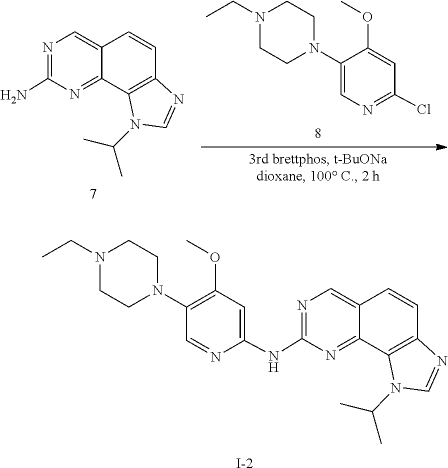

To a solution of 8 (148 mg, 579 μmol, 1.00 eq) in dioxane (3 mL) was added 7 (131 mg, 579 μmol, 1.00 eq), t-BuONa (111 mg, 1.16 mmol, 2.00 eq) and BrettPhos Pd G3 (53 mg, 57.9 μmol, 0.10 eq). The resulting mixture was stirred for 2 h at 100° C. under nitrogen atmosphere. The suspension was diluted with DCM (10 mL) and filtered. The filter cake was washed with DCM (10 mL). The filtrate was concentrated under vacuum. The residue was purified by silica gel column chromatography eluted with DCM/MeOH (10/1) to afford the crude (150 mg) as a faint yellow solid. The crude was purified by Prep-TLC to afford the target I-2 (50 mg, 20.0%) as an off-white solid. NH-NMR (DMSO-d₆, 400 MHz), 9.82 (s, 1H), 9.34 (s, 1H), 8.60 (s, 1H), 7.83 (s, 1H), 7.69-7.63 (m, 3H), 6.02-5.96 (n, 14), 3.91 (s, 3H), 3.13-3.07 (i, 4H), 2.82-2.77 (m, 4H), 2.67-2.64 (m, 2H), 1.56 (d, J=6.0 Hz, 6H), 1.11 (t, J=7.2 Hz, 3H).

### Examples 2-18

Examples 2-18 were prepared according to the similar operations as used in Example 1 by using different S8.

| Ex. No. | Structure | Identification |
|---|---|---|
| 2 | 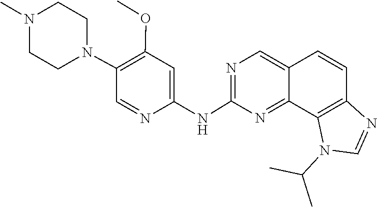 [SMILES: COc1cc(Nc2ncc3ccc4ncn(C(C)C)c4c3n2)ncc1N1CCN(C)CC1] | ¹H NMR (300 MHz, DMSO-d6) δ 9.75 (s, 1H), 9.34 (s, 1H), 8.59 (s, 1H), 7.81 (s, 1H), 7.73-7.61 (m, 3H), 6.04-5.93 (m, 1H), 3.91 (s, 3H), 3.07-2.97 (m, 4H), 2.57-2.47 (m, 4H), 2.24 (s, 3H), 1.56 (d, J = 6.6 Hz, 6H). |
| | I-1 | |
| 3 | 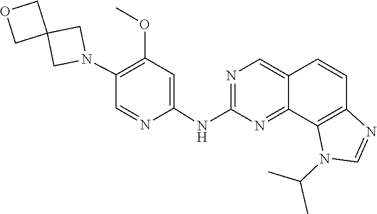 [SMILES: COc1cc(Nc2ncc3ccc4ncn(C(C)C)c4c3n2)ncc1N1CC2(COC2)C1] | ¹H NMR (300 MHz, DMSO-d6) δ 9.61 (s, 1H), 9.30 (s, 1H), 8.57 (s, 1H), 7.67-7.60 (m, 2H), 7.53 (s, 1H), 7.41 (s, 1H), 5.99-5.90 (m, 1H), 4.72 (s, 4H), 4.01 (s, 4H), 3.86 (s, 3H), 1.54 (d, J = 6.6 Hz, 6H). |
| | I-3 | |
| 4 | 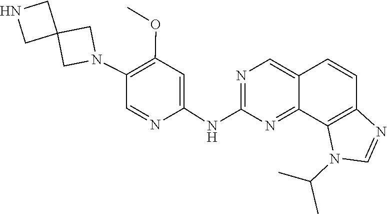 [SMILES: COc1cc(Nc2ncc3ccc4ncn(C(C)C)c4c3n2)ncc1N1CC2(CNC2)C1] | ¹H NMR (300 MHz, DMSO-d6) δ 9.64 (s, 1H), 9.31 (s, 1H), 8.58 (s, 1H), 7.68-7.55 (m, 2H), 7.55 (s, 1H), 7.42 (s, 1H), 6.00-5.93 (m, 1H), 4.15 (s, 4H), 4.01 (s, 4H), 3.87 (s, 3H), 1.55 (d, J = 6.6 Hz, 6H). |
| | I-4 | |
| 5 | 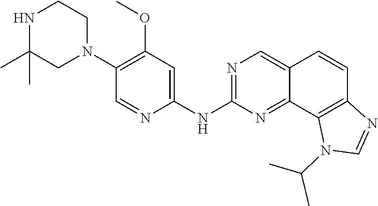 [SMILES: COc1cc(Nc2ncc3ccc4ncn(C(C)C)c4c3n2)ncc1N1CCNC(C)(C)C1] | ¹H NMR (300 MHz, DMSO-d6) δ 9.80 (s, 1H), 9.35 (s, 1H), 8.60 (s, 1H), 7.83 (s, 1H), 7.71-7.64 (m, 3H), 6.03-5.98 (m, 1H), 3.92 (s, 3H), 3.18-3.12 (m, 2H), 3.12-3.06 (m, 2H), 2.87 (s, 2H), 1.57 (d, J = 6.9 Hz, 6H), 1.32 (s, 6H). |
| | I-5 | |
| 6 | 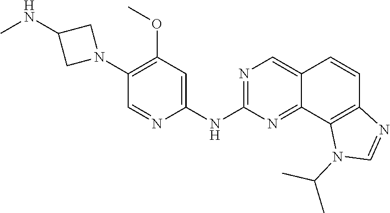 [SMILES: CNC1CN(c2cnc(Nc3ncc4ccc5ncn(C(C)C)c5c4n3)cc2OC)C1] | ¹H NMR (300 MHz, DMSO-d6) δ 9.34 (s, 1H), 9.04 (s, 1H), 8.60 (s, 1H), 7.68-7.63 (m, 2H), 7.57 (s, 1H), 7.50 (s, 1H), 5.99-5.92 (m, 1H), 4.17-4.11 (m, 2H), 4.08-4.00 (m, 2H), 3.93-3.90 (m, 4H), 2.62 (s, 3H), 1.57 (d, J = 6.6 Hz, 3H). |
| | I-6 | |
| 7 | 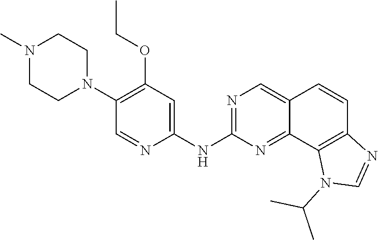 [SMILES: CCOc1cc(Nc2ncc3ccc4ncn(C(C)C)c4c3n2)ncc1N1CCN(C)CC1] | ¹H NMR (300 MHz, DMSO-d6) δ 9.72 (s, 1H), 9.33 (s, 1H), 8.59 (s, 1H), 7.80 (s, 1H), 7.69-7.65 (m, 2H), 7.63 (s, 1H), 6.01-5.95 (m, 1H), 4.16 (q, J = 6.9 Hz, 2H), 3.07-3.00 (m, 4H), 2.53 (s, 3H), 2.28-2.22 (m, 4H), 1.56 (d, J = 6.6 Hz, 6H), 1.42 (t, J = 6.9 Hz, 3H). |
| | I-7 | |
| 8 | 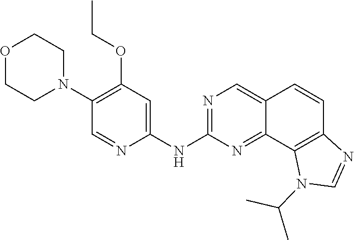 [SMILES: CCOc1cc(Nc2ncc3ccc4ncn(C(C)C)c4c3n2)ncc1N1CCOCC1] | ¹H NMR (300 MHz, DMSO-d6) δ 9.90 (br, 1H), 9.36 (s, 1H), 8.61 (s, 1H), 7.81 (s, 1H), 7.72-7.65 (m, 2H), 7.60 (s, 1H), 6.04-5.93 (m, 1H), 4.18 (q, J = 6.6 Hz, 2H), 3.76 (t, J = 4.8 Hz, 4H), 3.03 (t, J = 4.8 Hz, 4H), 1.58 (d, J = 6.6 Hz 6H), 1.43 (t, J = 6.9 Hz, 3H). |
| | I-8 | |
| 9 | 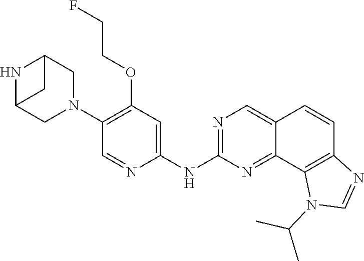 [SMILES: CC(C)n1cnc2ccc3cnc(Nc4cc(OCCF)c(N5CC6CC(C5)N6)cn4)nc3c21] | ¹H NMR (300 MHz, DMSO-d6) δ 9.70 (br, 1H), 9.32 (s, 1H), 8.58 (s, 1H), 7.95 (s, 1H), 7.69-7.62 (m, 3H), 6.00-5.96 (m, 1H), 4.89 (d, J = 4.5 Hz, 1H), 4.72 (d, J = 4.2 Hz, 1H), 4.40 (s, 1H), 4.30 (s, 1H), 3.68-3.64 (m, 2H), 3.58 (d, J = 5.4 Hz, 2H), 3.46 (d, J = 10.5 Hz, 2H), 2.42 (d, J = 6.6 Hz, 1H), 1.86 (d, J = 7.8 Hz, 1H), 1.57 (d, J = 6.6 Hz, 6H). |
| | I-9 | |
| 10 | 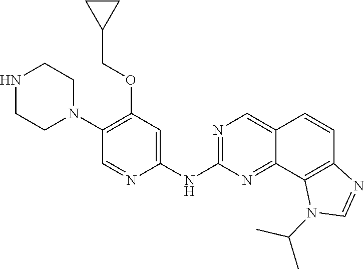 [SMILES: CC(C)n1cnc2ccc3cnc(Nc4cc(OCC5CC5)c(N5CCNCC5)cn4)nc3c21] | ¹H NMR (300 MHz, DMSO-d₆) δ 9.70 (s, 1H), 9.33 (s, 1H), 8.59 (s, 1H), 7.78 (s, 1H), 7.69-7.59 (m, 3H), 5.99-5.95 (m, 1H), 3.99 (d, J = 6.6 Hz, 2H), 3.44-3.40 (m, 4H), 2.90-2.87 (m, 4H), 1.57 (d, J = 6.9 Hz, 6H), 1.30-1.24 (m, 1H), 0.62-0.59 (m, 2H), 0.41-0.38 (m, 2H). |
| | I-10 | |
| 11 | 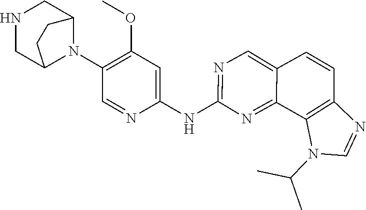 [SMILES: COc1cc(Nc2ncc3ccc4ncn(C(C)C)c4c3n2)ncc1N1C2CCC1CNC2] | ¹H NMR (300 MHz, DMSO-d₆) δ 9.65 (s, 1H), 9.32 (s, 1H), 8.58 (s, 1H), 7.76 (s, 1H), 7.68-7.59 (m, 2H), 7.55 (s, 1H), 5.99-5.92 (m, 1H), 3.99-3.94 (m, 2H), 3.90 (s, 3H), 3.01 (d, J = 11.7 Hz, 2H), 2.61 (d, J = 12.0 Hz, 2H), 1.90-1.85 (m, 4H), 1.55 (d, J = 6.9 Hz, 6H). |
| | I-11 | |
| 12 | 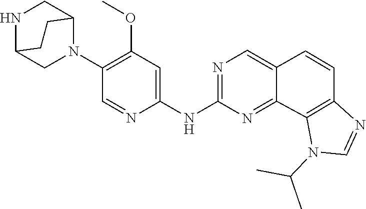 [SMILES: COc1cc(Nc2ncc3ccc4ncn(C(C)C)c4c3n2)ncc1N1CC2CCC1CN2] | ¹H NMR (300 MHz, DMSO-d₆) δ 9.66 (s, 1H), 9.32 (s, 1H), 8.58 (s, 1H), 7.78 (s, 1H), 7.68-7.59 (m, 3H), 6.02-5.93 (m, 1H), 3.89 (s, 3H), 3.61-3.53 (m, 4H), 3.13-3.02 (m, 2H), 2.10-2.05 (m, 1H), 1.90-1.77 (m, 3H), 1.55 (d, J = 6.6 Hz, 6H). |
| | I-12 | |
| 13 | 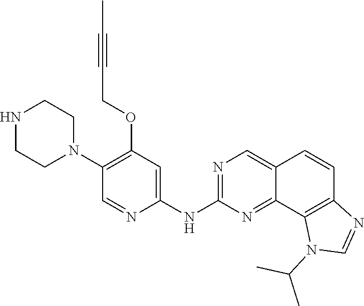 | ¹H NMR (300 MHz, DMSO-d₆) δ 9.79 (s, 1H), 9.33 (s, 1H), 8.59 (s, 1H), 7.84 (s, 1H), 7.75 (s, 1H), 7.70- 7.63 (m, 2H), 6.03-5.98 (m, 1H), 4.90 (d, J = 2.4 Hz, 2H), 2.96-2.93 (m, 4H), 2.90-2.86 (m, 4H), 1.82 (s, 3H), 1.58 (d, J = 6.6 Hz, 6H). |
| | I-13 | |
| 14 | 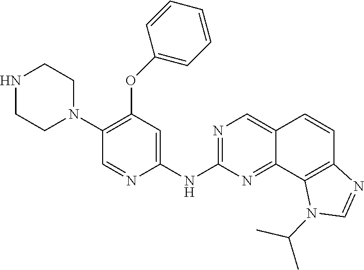 [SMILES: CC(C)n1cnc2ccc3cnc(Nc4cc(Oc5ccccc5)c(N5CCNCC5)cn4)nc3c21] | ¹H NMR (300 MHz, DMSO-d₆) δ 10.15 (s, 1H), 9.29 (s, 1H), 8.56 (s, 1H), 8.14 (s, 1H), 7.99 (s, 1H), 7.70- 7.64 (m, 2H), 7.45-7.40 (m, 2H), 7.20-7.15 (m, 1H), 7.08-7.05 (m, 2H), 5.90-5.86 (m, 1H), 3.23-3.19 (m, 4H), 2.87-2.85 (m, 4H), 1.46 (d, J = 6.6 Hz, 6H). |
| | I-14 | |
| 15 | 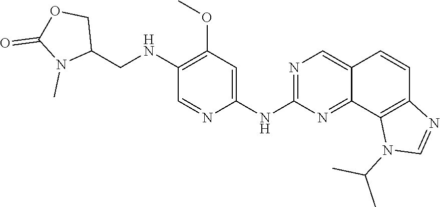 [SMILES: COc1cc(Nc2ncc3ccc4ncn(C(C)C)c4c3n2)ncc1NCC1COC(=O)N1C] | ¹H NMR (300 MHz, DMSO-d₆) δ 11.67 (s, 1H), 9.59 (s, 1H), 9.05 (s, 1H), 8.00 (d, J = 9 Hz, 1H), 7.88 (d, J = 9 Hz, 2H), 7.64 (s, 1H), 7.29 (s, 1H), 6.03-5.94 (m, 2H), 4.38 (t, J = 8.7 Hz, 1H), 4.14-3.99 (m, 5H), 3.47-3.41 (m, 1H), 3.33-3.26 (m, 1H), 2.86 (s, 3H), 1.68 (d, J = 6.6 Hz, 3H). |
| | I-15 | |
| 16 | 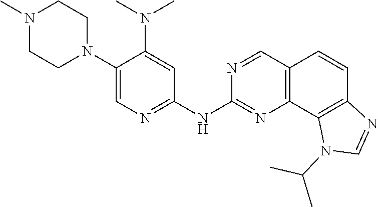 [SMILES: CC(C)n1cnc2ccc3cnc(Nc4cc(N(C)C)c(N5CCN(C)CC5)cn4)nc3c21] | |
| | I-16 | |
| 17 | 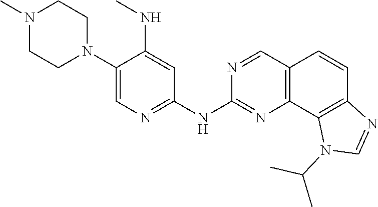 [SMILES: CNc1cc(Nc2ncc3ccc4ncn(C(C)C)c4c3n2)ncc1N1CCN(C)CC1] | |
| | I-17 | |
| 18 | 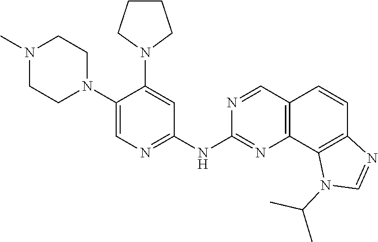 [SMILES: CC(C)n1cnc2ccc3cnc(Nc4cc(N5CCCC5)c(N5CCN(C)CC5)cn4)nc3c21] | |
| | I-18 | |

### Example 19: Preparation of 1-isopropyl-N-(3-methoxy-4-morpholinophenyl)-4-methyl-1H-imidazo[4,5-h]quinazolin-8-amine (IT-1)

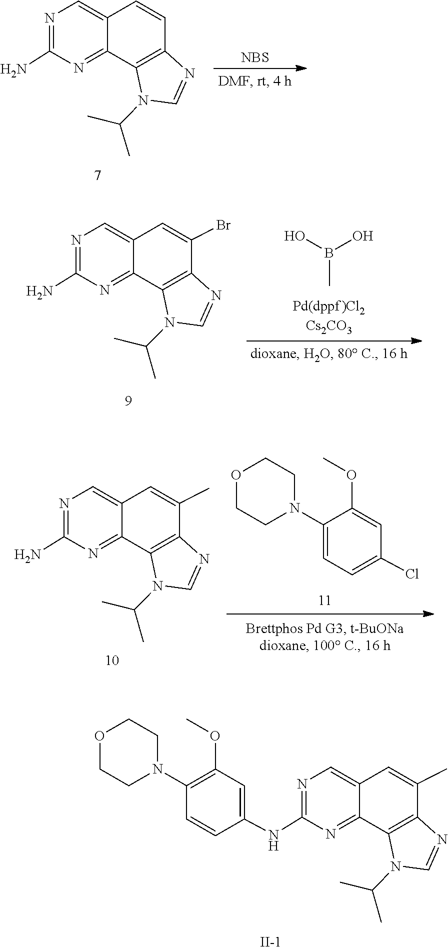

Step 1

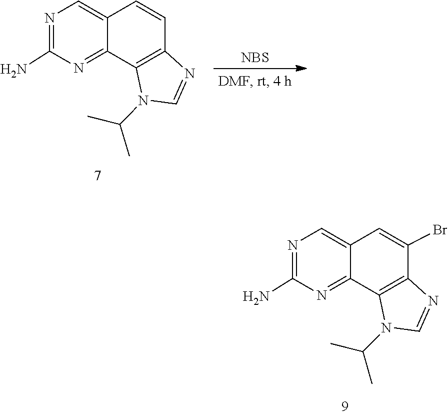

To a solution of 7 (200 mg, 880 μmol, 1.00 eq) in DMF (10 mL) was added N-bromosuccinimide (235 mg, 1.32 mmol, 1.50 eq). The resulting reaction was stirred for 4 h at room temperature. The suspension was diluted with EtOAc (40 mL, washed with brine (10 mL×3). The combined organic layers were dried over anhydrous Na₂SO₄ and filtered, and the filter cake was washed with EtOAc (10 mL). The filtrate was concentrated in vacuum. The residue was purified by silica gel column chromatography eluted with EtOAc to afford the compound 9 (240 mg, 89.1%) as an off-white solid. LCMS [M+1]+=306.1, and 308.0.

Step 2

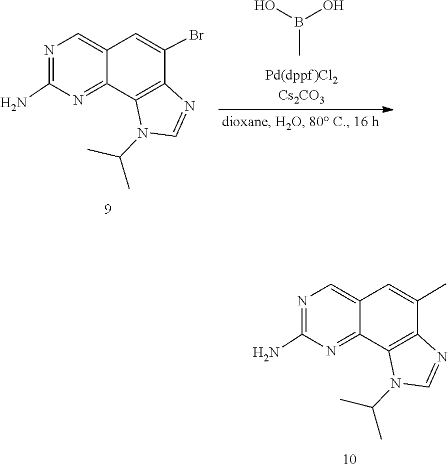

To a solution of 9 (240 mg, 784 μmol, 1.00 eq) in dioxane (16 mL) and H₂O (4 mL) was added methylboronic acid (469 mg, 7.84 mmol, 10.0 eq), Cs₂CO₃ (769 mg, 2.35 mmol, 3.00 eq) and Pd(dppf)Cl₂ (57 mg, 78.4 μmol, 0.10 eq). The resulting mixture was stirred for 16 h at 80° C. under nitrogen atmosphere. The suspension was diluted with water (20 mL), and extracted with EtOAc (10 mL×3). The combined organic layers were dried over anhydrous Na₂SO₄ and filtered. The filtrate was concentrated in vacuum. The residue was purified by silica gel column chromatography eluted with EtOAc to afford the compound 10 (82 mg, 43.4%) as an off-white solid. LCMS [M+1]⁺=242.2.

Step 3

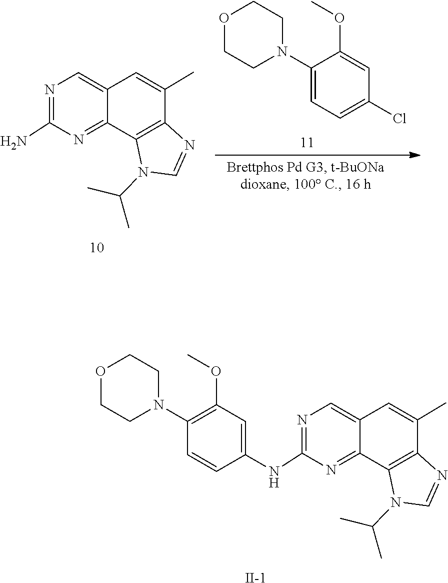

To a solution of 11 (77 mg, 337 μmol, 1.00 eq) in dioxane (5 mL) was added 10 (81 mg, 337 μmol, 1.0 eq), t-BuONa (81 mg, 842 μmol, 2.50 eq) and Brettphos Pd G3 (31 mg, 33.7 μmol, 0.10 eq). The resulting mixture stirred for 16 h at 100° C. under nitrogen atmosphere. The mixture was concentrated in vacuum. The residue was purified by silica gel column chromatography eluted with DCM/MeOH (10/1) to afford the title compound (40 mg, 27.4%) as a yellow solid. LCMS [M+1]⁺=434.3. ¹H NMR (300 MHz, DMSO-d6) δ 9.66 (s, 1H), 9.25 (s, 1H), 8.57 (s, 1H), 7.81 (s, 1H), 7.68 (s, 1H), 7.46 (d, J=1.2 Hz, 1H), 6.05-5.96 (m, 1H), 3.92 (s, 3H), 3.76-3.73 (m, 4H), 3.02-2.98 (m, 4H), 2.61 (s, 3H), 1.57 (d, J=6.6 Hz, 6H).

### Example 20: Preparation of N-(4-ethoxy-5-(piperazin-1-yl)pyridin-2-yl)-1-isopropyl-5-methyl-1H-imidazo[4,5-h]quinazolin-8-amine (III-1)

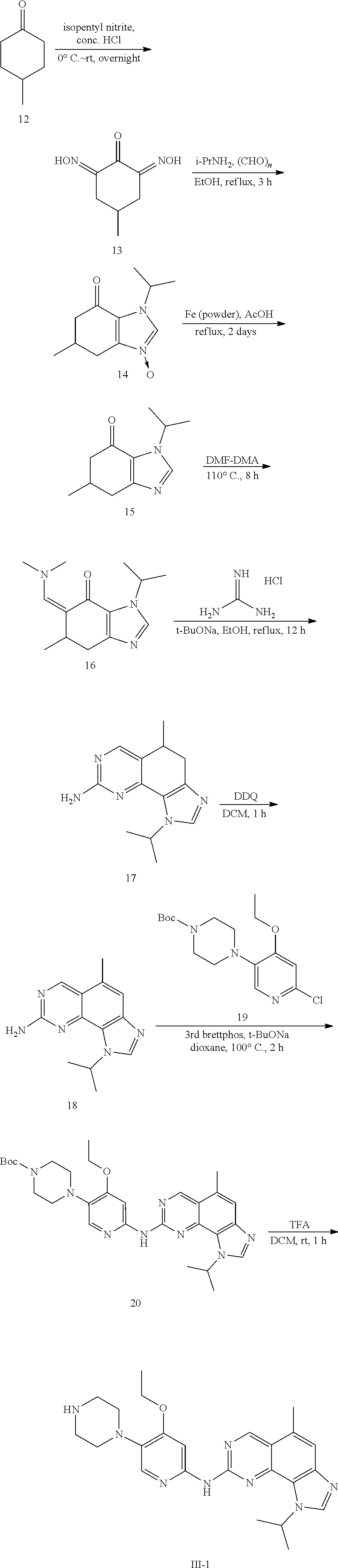

Step 1

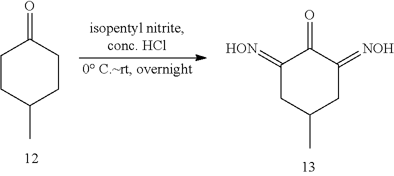

To 12 (20.0 g, 178 mmol, 1.00 eq) was added conc. HCl (1.5 mL, 12 M, 17.8 mmol, 0.10 eq), followed by isopentyl nitrite (41.7 g, 357 mmol, 2.00 eq) at 5° C. Then the mixture was allowed to warm to room temperature and stirred for 14 h. The solids were filtered out. The filter cake was washed with PE (300 mL×3), dried in vacuum to afford the compound 13 (28.8 g, 94.9%) as a pale yellow solid.

Steps 2 and 3

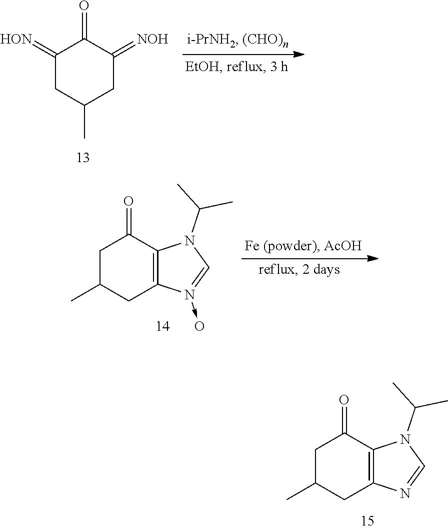

To a solution of 13 (28.8 g, 169 mmol, 1.00 eq) in EtOH (500 mL) was added polyoxymethylene (5.58 g, 186 mmol, 1.10 eq), isopropyl amine (11.0 g, 186 mmol, 1.10 eq). The mixture was stirred at room temperature for 1 h and then refluxed for another 2 h. The mixture was concentrated to afford the crude compound 14.

To a solution of crude compound 14 in AcOH (500 mL) was added Fe (powder) (66.2 g, 1.18 mol, 7.00 eq). The resulting mixture was refluxed for 48 h. After cooled to room temperature, the mixture was diluted with EtOAc (500 mL) and filtered. The filtrate was concentrated in vacuum. The residue was suspended in EtOAc (1000 mL) and saturated sodium bicarbonate (500 mL). The solids were filtered out. The organic layer was separated and the aqueous was extracted with DCM/MeOH (10/1, 500 mL×2). The combined organic layers were dried over Na₂SO₄ and concentrated in vacuum. The residue was purified by silica gel column chromatography eluted with PE/EtOAc (1/2) to afford the compound 15 (23.1 g, two steps 76.6%) as a brown oil. LCMS [M+H]⁺=193.2.

Steps 4 and 5

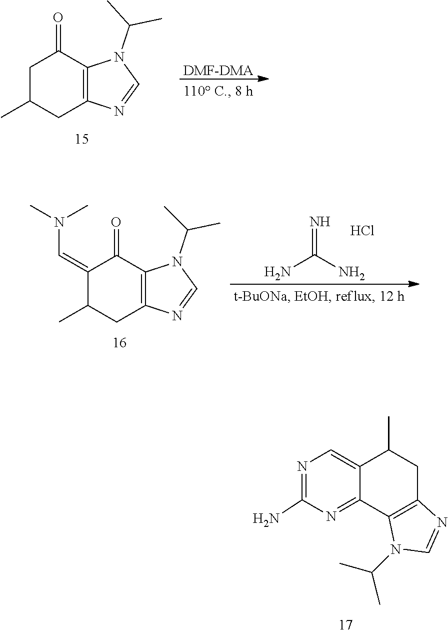

To a solution of 15 (23.1 g, 120 mmol, 1.00 eq) in DMF (250 mL) was added DMF-DMA (250 mL). The resulting solution was stirred for 8 h at 110° C. The solvent was removed in vacuum to give the crude compound 16 (30.0 g) which was used in the next step directly without further purification.

To a solution of the crude compound 16 (30.0 g) in EtOH (300 mL) was added guanidine hydrochloride (11.5 g, 120 mmol, 1.00 eq) and t-BuONa (34.6 g, 360 mmol, 3.00 eq). The resulting mixture was stirred for 12 h at 80° C. The solvent was removed in vacuum. The residue was purified by silica gel column chromatography eluted with PE/EtOAc (1/4) to afford the compound 17 (9.70 g, two steps 33.2%) as a yellow solid. LCMS [M+H]⁺=244.3.

Step 6

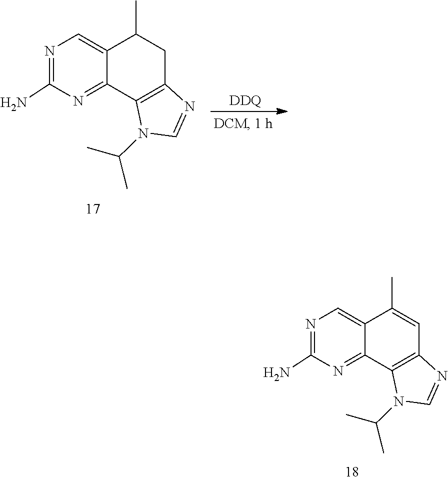

To a solution of 17 (9.70 g, 39.9 mmol, 1.00 eq) in DCM (400 mL) was added DDQ (10.9 g, 48 mmol, 1.20 eq). The resulting mixture was stirred for 1 h at room temperature. The mixture solution was concentrated in vacuum. The residue was purified by silica gel column chromatography eluted with PE/EtOAc (1/1) to afford the compound 18 (800 mg, 8.32%) and 5.1 g (crude) as a light yellow solid. LCMS [M+H]⁺=242.2.

Step 7

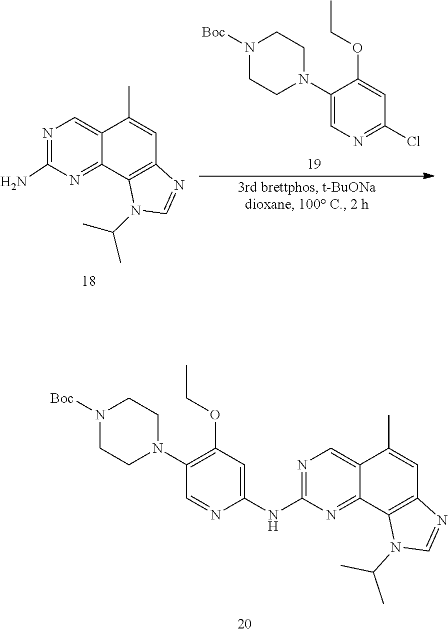 [SMILES: Cc1cc2ncn(C(C)C)c2c2nc(N)ncc12 ; CCOc1cc(Cl)ncc1N1CCN(C(=O)OC(C)(C)C)CC1 ; CCOc1cc(Nc2ncc3c(C)cc4ncn(C(C)C)c4c3n2)ncc1N1CCN(C(=O)OC(C)(C)C)CC1]

To a solution of 19 (63 mg, 184 μmol, 1.00 eq) in dioxane (3 mL) was added 18 (44 mg, 184 μmol, 1.00 eq), t-BuONa (35 mg, 369 μmol, 2.00 eq) and Brettphos Pd G3 (33 mg, 36.9 μmol, 0.20 eq). The resulting mixture stirred for 2 h at 110° C. under nitrogen atmosphere. The mixture was concentrated in vacuum. The residue was purified by prep-TLC eluted with EtOAc to afford the compound 20 (63 mg, 62.5%) as a light yellow solid. LCMS [M+H]⁺=547.4.

Step 8

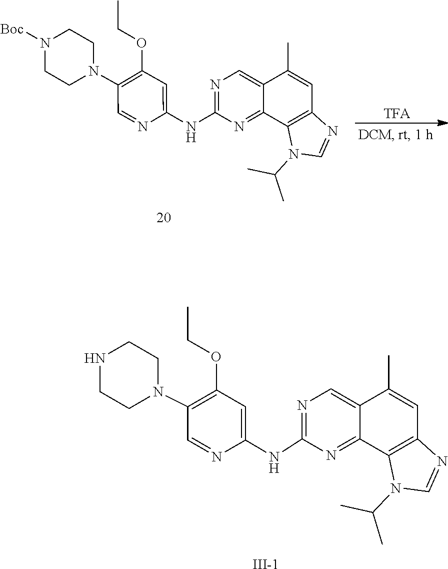 [SMILES: CCOc1cc(Nc2ncc3c(C)cc4ncn(C(C)C)c4c3n2)ncc1N1CCN(C(=O)OC(C)(C)C)CC1 ; CCOc1cc(Nc2ncc3c(C)cc4ncn(C(C)C)c4c3n2)ncc1N1CCNCC1 ; "III-1"]

To a solution of 20 (63 mg, 115 μmol, 1.00 eq) in DCM (5 mL) was added TFA (1.0 mL). The resulting mixture was stirred for 1 h at room temperature. The solvent was concentrated in vacuum to afford the crude product. The crude product was suspended in MeOH (5 mL) and saturated sodium carbonate aqueous (2 mL) and stirring for 10 min. The solids were filtered out. The filtrate was concentrated in vacuum. The residue was purified by prep-TLC eluted with DCM/MeOH (10/1) to afford the target III-1 (20 mg, 38.9%) as an off-white solid. LCMS [M+H]⁺=447.3. ¹H NMR (300 MHz, DMSO-d₆) δ 9.79 (s, 1H), 9.42 (s, 1H), 8.51 (s, 1H), 7.84 (s, 1H), 7.71 (s, 1H), 7.44 (d, J=1.2 Hz, 1H), 6.01-5.97 (m, 1H), 4.17 (q, J=6.9 Hz, 2H), 3.16-3.11 (m, 8H), 2.71 (s, 3H), 1.54 (d, J=6.6 Hz, 6H), 1.43 (t, J=6.9 Hz, 3H).

### Example 21: Preparation of N-(4-isopropoxy-5-(piperazin-1-yl)pyridin-2-yl)-1-methyl-1H-imidazo[4,5-h]quinazolin-8-amine (IV-1)

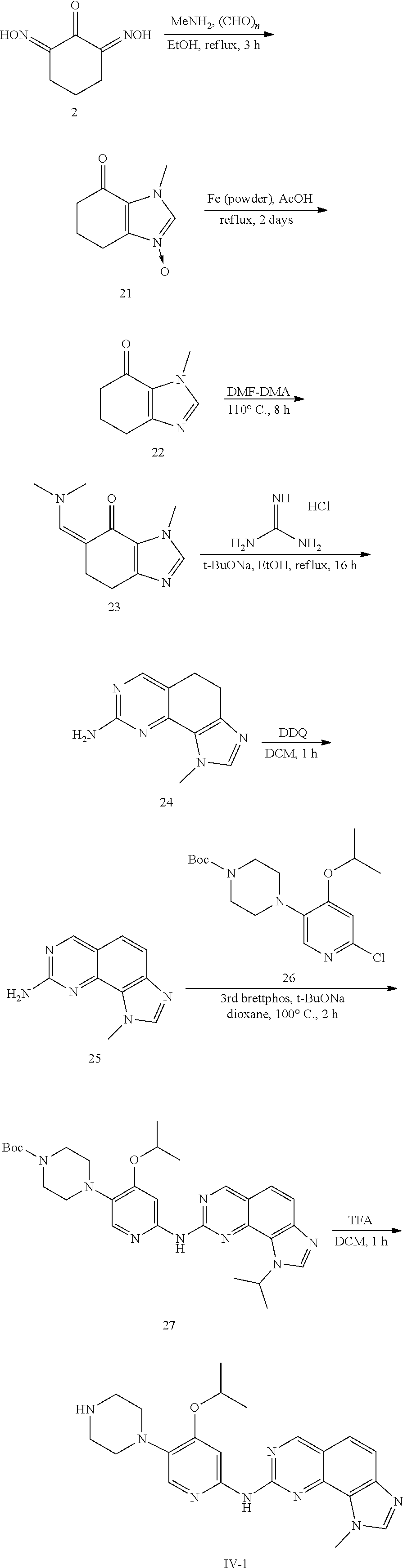

Steps 1 and 2

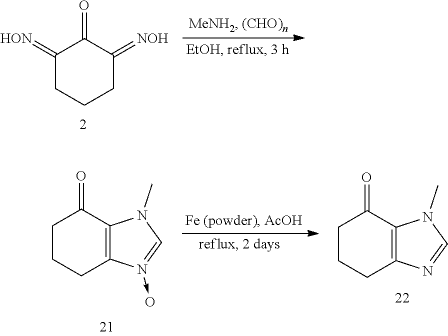

To a solution of 2 (21.0 g, 135 mmol, 1.00 eq) in EtOH (300 mL) was added polyoxymethylene (4.44 g, 148 mmol, 1.10 eq), methylamine (74 mL, 2M in THF, 148 mmol, 1.10 eq). The resulting mixture was stirred at room temperature for 1 h and then refluxed for another 2 h. The mixture was concentrated to afford the crude compound 21.

To a solution of crude compound 21 in AcOH (300 mL) was added Fe (powder) (37.5 g, 672 mol, 5.00 eq). The resulting mixture was stirred for 48 h at 120° C. After cooled to room temperature, the mixture was diluted with EtOAc (500 mL) and filtered. The filtrate was concentrated. The residue was suspended in EtOAc (500 mL) and saturated sodium bicarbonate (300 mL), filtered. The organic layer was separated and the aqueous was extracted with DCM/MeOH (10/1, 500 mL×2). The combined organic layers were dried over Na₂SO₄ and concentrated in vacuum. The residue was purified by silica gel column chromatography eluted with EtOAc to afford the compound 22 (15.8 g, 78.2%) as a brown oil. LCMS [M+H]⁺=151.1.

Steps 3 and 4

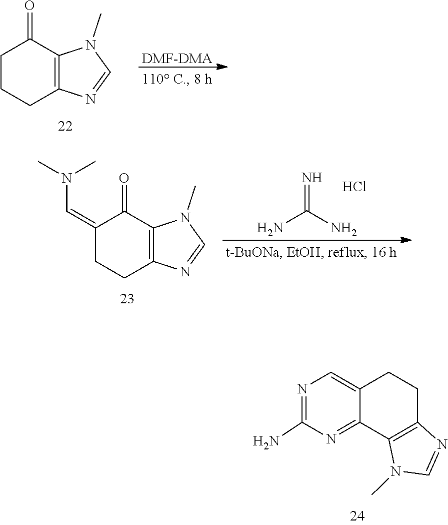

To a solution of 22 (15.8 g, 105 mmol, 1.00 eq) in DMF (45 mL) was added DMF-DMA (45 mL). The resulting solution was stirred for 8 h at 110° C. The solvent was removed in vacuum to give the crude product 23 which was used in the next step directly without further purification. LCMS [M+H]⁺=205.2.

To a solution of the crude product 23 in EtOH (300 mL) was added guanidine hydrochloride (25.1 g, 263 mmol, 2.50 eq) and t-BuONa (50.6 g, 526 mmol, 5.00 eq). The resulting mixture was stirred for 16 h at 70° C. The solvent was removed in vacuum. The residue was purified by silica gel column chromatography eluted with EtOAc to afford the compound 24 (6.30 g, 29.8%) as a yellow solid. LCMS [M+H]⁺=202.2.

Step 5

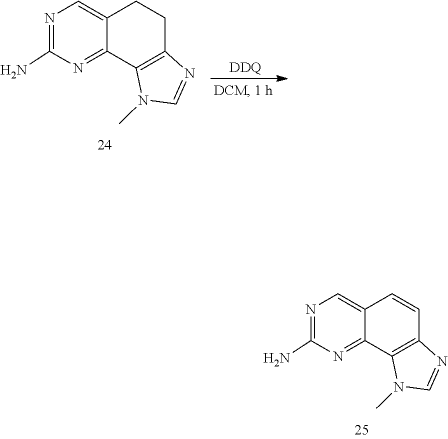

To a solution of 24 (3.0 g, 14.9 mmol, 1.00 eq) in DCM (100 mL) was added DDQ (4.1 g, 18 mmol, 1.20 eq). The resulting mixture was stirred for 1 h at room temperature. The mixture solution was concentrated in vacuum. The residue was purified by silica gel column chromatography eluted with PE/EtOAc (1/1) to afford the compound 25 (1.20 g, 40.4%) as a yellow solid. LCMS [M+H]⁺=200.1.

Step 6

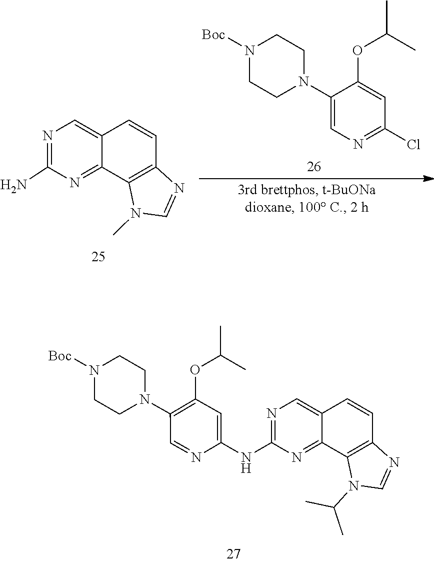 [SMILES: Cn1cnc2ccc3cnc(N)nc3c21 ; CC(C)Oc1cc(Cl)ncc1N1CCN(C(=O)OC(C)(C)C)CC1 ; CC(C)Oc1cc(Nc2ncc3ccc4ncn(C(C)C)c4c3n2)ncc1N1CCN(C(=O)OC(C)(C)C)CC1]

To a solution of 26 (109 mg, 306 μmol, 1.00 eq) in dioxane (5 mL) was added 25 (61 mg, 306 μmol, 1.00 eq), t-BuONa (60 mg, 613 μmol, 2.00 eq) and Brettphos Pd G3 (14 mg, 15.3 μmol, 0.05 eq). The resulting mixture was stirred for 2 h at 100° C. under nitrogen atmosphere. The mixture was concentrated. The residue was purified by silica gel column chromatography eluted with EtOAc to afford the compound 27 (92 mg, 57.9%) as a yellow solid. LCMS [M+H]⁺=519.3.

Step 7

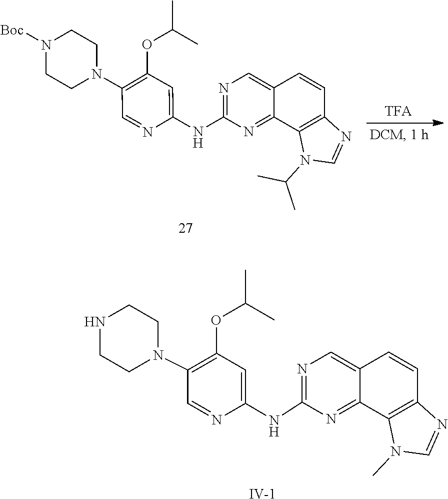 [SMILES: CC(C)Oc1cc(Nc2ncc3ccc4ncn(C(C)C)c4c3n2)ncc1N1CCN(C(=O)OC(C)(C)C)CC1 ; CC(C)Oc1cc(Nc2ncc3ccc4ncn(C)c4c3n2)ncc1N1CCNCC1 ; "IV-1"]

To a solution of 27 (92 mg, 177 μmol, 1.00 eq) in DCM (5 mL) was added TFA (0.1 mL). The resulting mixture was stirred for 1 h at room temperature. The mixture was concentrated in vacuum to afford the crude product. To a suspension of the crude product in MeOH (2 mL) was added Na₂CO₃ (57 mg, 0.48 mmol, 3.00 eq) with stirring for 10 min, followed by DCM (10 mL). The solids were filtered out. The filtrate was concentrated and the residue was purified by prep-TLC to afford the title compound IV-1 (19.4 mg, 26.1%). LCMS [M+H]⁺=419.3. ¹H NMR (300 MHz, DMSO-d₆) δ 9.64 (s, 1H), 9.33 (s, 1H), 8.35 (s, 1H), 7.80 (s, 2H), 7.69-7.62 (m, 2H), 4.86-4.77 (m, 1H), 4.42 (s, 3H), 2.95-2.91 (m, 4H), 2.85-2.82 (m, 4H), 1.35 (d, J=6.0 Hz, 6H).

### Example 22: Preparation of N-(4-ethoxy-5-(4-ethylpiperazin-1-yl)pyridin-2-yl)-1-ethyl-1H-imidazo[4,5-h]quinazolin-8-amine (V-1)

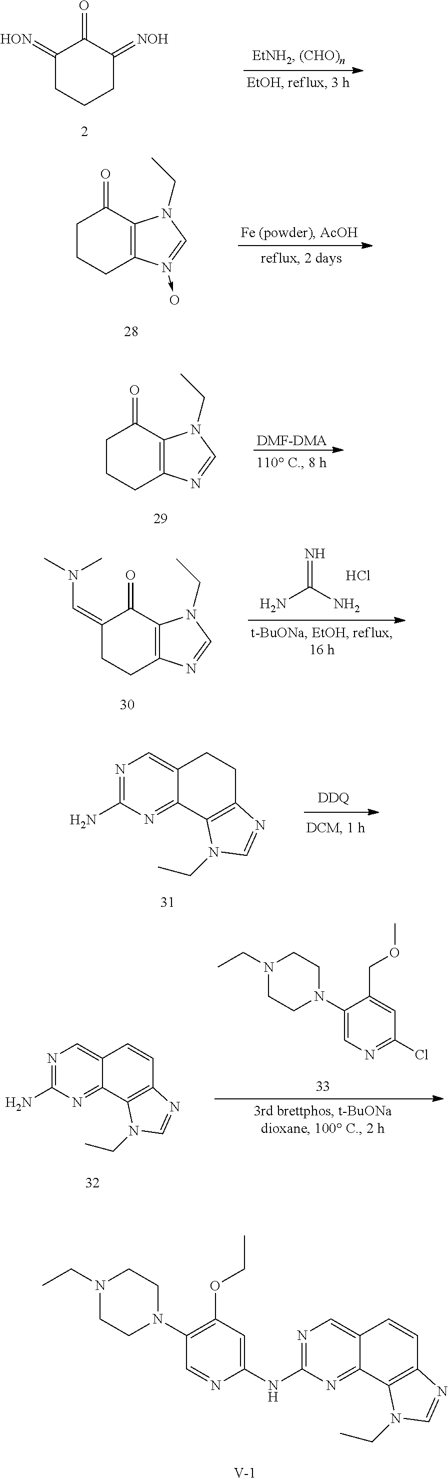

Steps 1 and 2

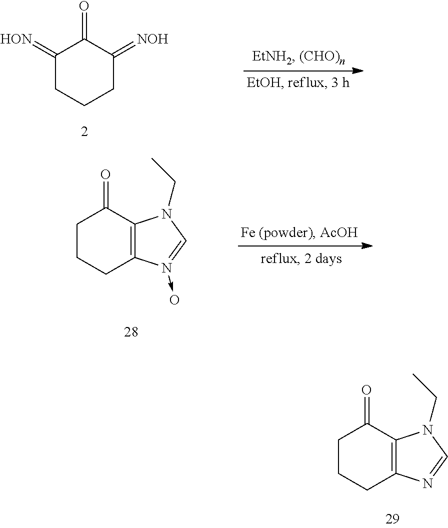

To a solution of 2 (21.0 g, 135 mmol, 1.00 eq) in EtOH (300 mL) was added polyoxymethylene (4.44 g, 148 mmol, 1.10 eq), ethylamine (6.67 g, 148 mmol, 1.10 eq). The resulting solution was stirred at room temperature for 1 h and then refluxed for another 2 h. The mixture was concentrated in vacuum to afford the crude compound 28 which was used in the next step directly without further purification. LCMS [M+H]⁺=181.1.

To a solution of crude compound 28 in AcOH (300 mL) was added Fe (powder) (37.6 g, 672 mmol, 5.0 eq). The resulting mixture was refluxed for 48 h. After cooled to room temperature, the mixture was diluted with EtOAc (500 mL) and filtered. The filtrate was concentrated. The residue was suspended in EtOAc (500 mL) and saturated sodium bicarbonate (300 mL). The mixture was filtered. The organic layer was separated and the aqueous was extracted with DCM/MeOH (10/1, 500 mL×2). The combined organic layers were dried over Na₂SO₄ and concentrated in vacuum. The residue was purified by silica gel column chromatography eluted with EtOAc to afford the compound 29 (14.3 g, two steps 64.7%) as a brown oil. LCMS [M+H]⁺=165.1.

Steps 3 and 4

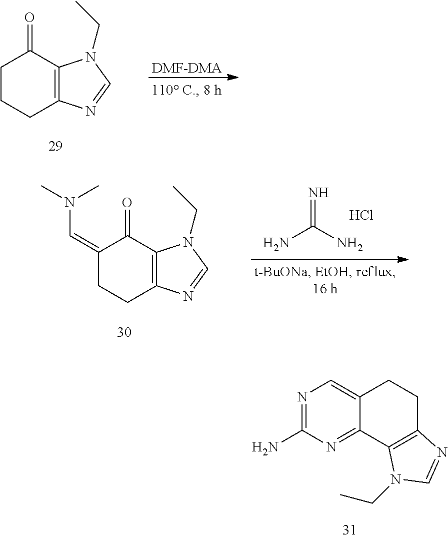

To a solution of 29 (14.3 g, 87.1 mmol, 1.00 eq) in DMF (42 mL) was added DMF-DMA (42 mL). The resulting solution was stirred for 8 h at 130° C. The solvent was removed in vacuum to give the crude product 30 which was used in the next step directly without further purification. LCMS [M+H]⁺=220.2.

To a solution of the crude product 30 in EtOH (300 mL) was added guanidine hydrochloride (8.32 g, 89.1 mmol, 1.00 eq) and t-BuONa (25.1 g, 261 mmol, 3.00 eq). The resulting mixture was refluxed for 16 h. The solvent was removed in vacuum. The residue was purified by silica gel column chromatography eluted with PE/EtOAc (1/1) to afford the compound 31 (6.4 g, 34.1%) as a yellow solid. LCMS [M+H]⁺=216.2.

Step 5

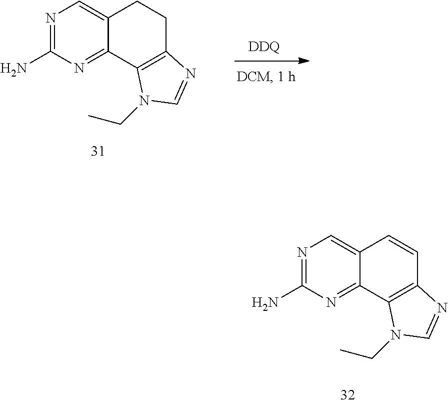

To a solution of 31 (6.40 g, 29.7 mmol, 1.00 eq) in DCM (200 mL) was added DDQ (8.09 g, 35.6 mmol, 1.20 eq). The resulting mixture was stirred for 1 h at room temperature. The mixture solution was concentrated in vacuum. The residue was purified by silica gel column chromatography eluted with PE/EtOAc (1/1) to afford the compound 32 (2.10 g, 33.1%) as a yellow solid. LCMS [M+H]⁺=214.2.

Step 6

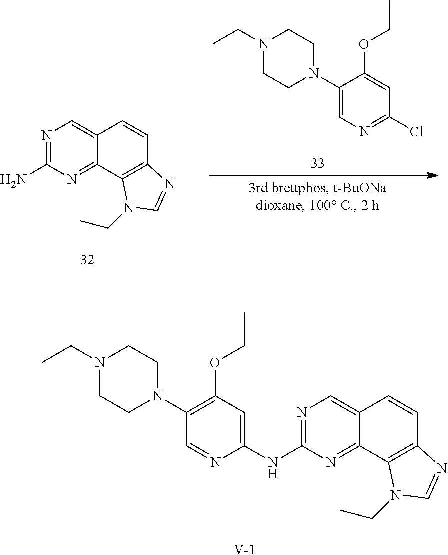

To a solution of 33 (87 mg, 323 μmol, 1.00 eq) in dioxane (5 mL) was added 32 (69 mg, 323 μmol, 1.00 eq), t-BuONa (62 mg, 645 μmol, 2.00 eq) and Brettphos Pd G3 (15 mg, 16.1 μmol, 0.05 eq). The resulting mixture was stirred for 2 h at 90° C. under nitrogen atmosphere. The solids were filtered out. The filtrate was concentrated. The residue was purified by silica gel column chromatography eluted with DCM/MeOH (15/1) to afford the target V-1 (21.2 mg, 14.6%). LCMS [M+H]⁺=447.4. ¹H NMR (300 MHz, DMSO-d₆) δ 9.69 (s, 1H), 9.33 (s, 1H), 8.43 (s, 1H), 7.81 (s, 1H), 7.70-7.63 (m, 3H), 4.88 (q, J=7.2 Hz, 2H), 4.17 (q, J=6.9 Hz, 2H), 3.09-2.59 (m, 4H), 2.59-2.56 (m, 2H), 2.43-2.36 (m, 4H), 1.46-1.39 (m, 6H), 1.05 (t, J=7.2 Hz, 3H).

### Example 23: Preparation of 1-isopropyl-N-(4-methoxy-5-morpholinopyridin-2-yl)-2-methyl-1H-imidazo[4,5-h]quinazolin-8-amine (VI-1)

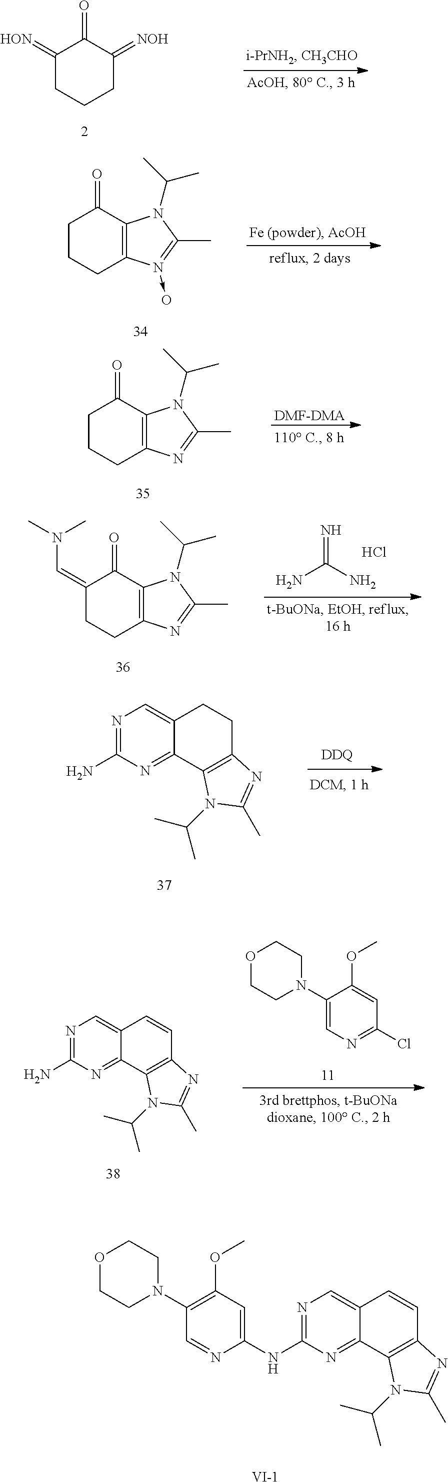

Steps 1 and 2

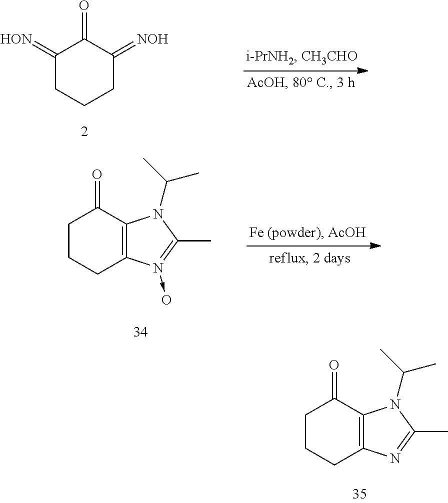

To a suspension of 2 (12.5 g, 80.1 mmol, 1.00 eq) in AcOH (150 mL) was added isopropyl amine (5.21 g, 88.1 mol, 1.10 eq) followed by CH₃CHO (3.88 g, 88.1 mmol, 1.10 eq). The resulting mixture was stirred for 3 h at 80° C. The mixture was used in the next step directly without further purification. LCMS [M+H]⁺=209.2.

After cooled to room temperature, Fe powder (22.4 g, 400 mmol, 5.00 eq) was added. The resulting mixture was refluxed for 48 h. The solvent was removed in vacuum. The residue was dilute with EtOAc (200 mL) and filtered. The filtrate was concentrated to afford the crude product. The crude product was poured into water (250 mL). The pH was adjusted to 8-9 with 1 N NaOH aqueous solution. The mixture was extracted with EtOAc (100 mL×3). The combined organic layers were dried over Na₂SO₄, filtered and concentrated. The residue was purified by silica gel column chromatography eluted with DCM/MeOH (20/1) to afford the compound 35 (10.5 g, two steps 68.2%) as a gray solid. LCMS [M+H]⁺=193.2.

Steps 3 and 4

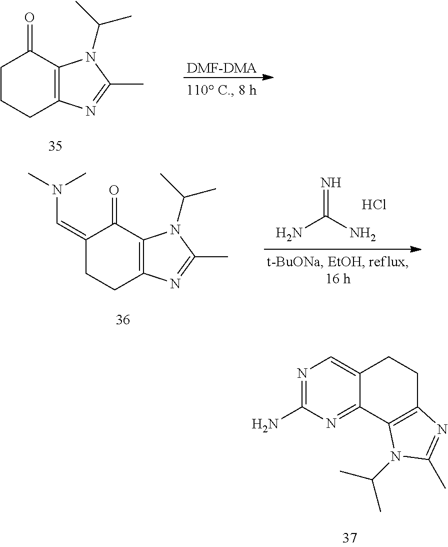

To a suspension of 35 (5.00 g, 26.0 mmol, 1.00 eq) in DMF (30 mL) was added DMF-DMA (30 mL). The resulting mixture was stirred for 8 h at 110° C. The mixture was concentrated in vacuum to afford the crude product 36.

To a solution of the crude product 36 in EtOH (150 mL) was added guanidine hydrochloride (6.15 g, 65.0 mmol, 2.50 eq), t-BuONa (12.4 g, 130 mmol, 5.00 eq). The resulting mixture was refluxed 16 h. The mixture was concentrated in vacuum. The residue was purified by silica gel column chromatography eluted with DCM/MeOH (20/1) to afford the compound 37 (1.50 g, two steps 23.7%) as a gray solid. LCMS [M+H]⁺=244.2.

Step 5

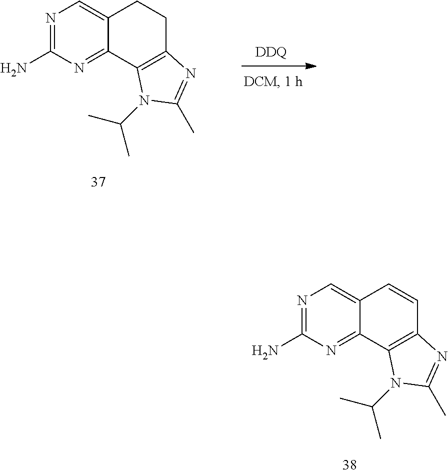

To a solution of 37 (200 mg, 822 μmol, 1.00 eq) in DCM (8 mL) was added DDQ (224 mg, 986 μmol, 1.20 eq). The resulting mixture was stirred for 1 h at room temperature. The mixture solution was concentrated in vacuum. The residue was purified by silica gel column chromatography eluted with PE/EtOAc (1/1) to afford the compound 38 (110 mg, 55.5%) as a brown solid. LCMS [M+H]⁺=242.2.

Step 6

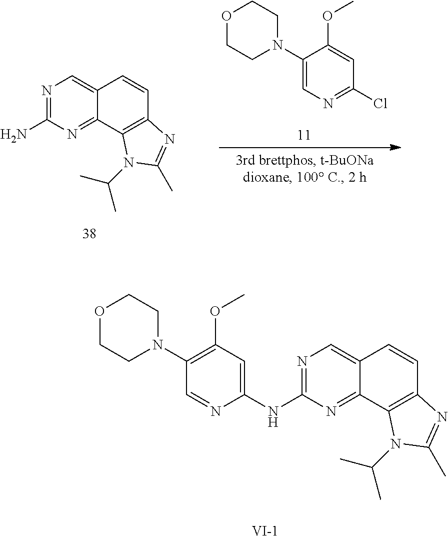 [SMILES: Cc1nc2ccc3cnc(N)nc3c2n1C(C)C ; COc1cc(Cl)ncc1N1CCOCC1 ; COc1cc(Nc2ncc3ccc4nc(C)n(C(C)C)c4c3n2)ncc1N1CCOCC1 ; "VI-1"]

To a suspension of 38 (100 mg, 414 μmol, 1.00 eq) in dioxane (10 mL) was added 11 (117 mg, 497 μmol, 1.20 eq), t-BuONa (80 mg, 829 μmol, 2.00 eq) and Brettphos Pd G3 (19 mg, 20.7 μmol, 0.05 eq). The resulting mixture was stirred for 2 h at 100° C. The mixture was concentrated in vacuum. The residue was purified by silica gel column chromatography eluted with PE/EtOAc (1/1) to afford the target VI-1 (22.4 mg, 12.5%) as a white solid. LCMS [M+H]⁺=434.3. ¹H NMR (300 MHz, DMSO-d₆) δ 9.73 (br, 1H), 9.30 (s, 1H), 7.82 (s, 1H), 7.64-7.59 (m, 2H), 7.52 (d, J=8.7 Hz, 1H), 3.89 (s, 3H), 3.76-3.73 (m, 4H), 3.08-3.00 (m, 4H), 2.69 (s, 3H), 1.60 (d, J=6.9 Hz, 6H).

Biological Assay

Compounds of the present disclosure were tested in a 10-dose IC50 mode with a 3-fold serial dilution starting from 0.5 μM, and the control compound Staurosporine was tested in a 10-dose IC50 mode with a 4-fold serial dilution starting from 20 μM. Reactions were carried out in the presence of 10 μM ATP.

Conditions and Protocol:

Procedures:
1. Compounds were prepared in freshly prepared reaction buffers containing 20 mM HEPES (pH 7.5), 10 mM MgCl₂, 1 mM EGTA, 0.01% Brij35, 0.02 mg/ml, BSA, 0.1 mM Na₃VO₄, 2 mM DTT, and 1% DMSO.
2. Required cofactor such as 1 μg (1.5 μM) of recombinant retinoblastoma protein in the case of CDK subtypes was added to the substrate solutions as mentioned above.
3. Kinase such as 10 ng of recombinant CDK4/cyclin D1 (Life Technologies PV4204) diluted in a kinase buffer (20 mM Tris pH7.5, 10 mM MgCl₂, 0.01% NP-40, 2 mM DTT), and incubated at room temperature for 30 minutes together with indicated concentration of inhibitors.
4. Compounds in DMSO were added into the kinase reaction mixture utilizing acoustic technology (Echo550).
5. 33P-ATP (specific activity 0.01 μCi/μl final) was added into the reaction mixture to initiate the reaction.
6. The reaction mixture was incubated for 120 minutes at room temperature.
7. Reactions are spotted onto P81 ion exchange paper (Whatman #3698-915).
8. Filters were extensively washed with 0.75% phosphoric acid.
9. The radioactive phosphorylated substrate remaining on the filter paper was measured.

Data Analysis:

Kinase activity data were expressed as the percent remaining kinase activity in test samples compared to vehicle (dimethyl sulfoxide) reactions. IC50 values and curve fits were obtained using Prism4 Software (GraphPad).

All the kinase inhibition results of the representative compounds using the similar assay methods as described above are shown in the following table.

| Compound | Flt3 IC50 | Flt3 (ITD) | C-Klt IC50 | CDK1/cyclin | CDK2/cyclin | CDK4/cyclin | CDK6/cyclin |
|---|---|---|---|---|---|---|---|
| No. | (nM) | IC50 (nM) | (nM) | B IC50 (nM) | E IC50 (nM) | IC50 (nM) | D1 IC50 (nM) |
| staurosporine | 1.55 | 1.56 | 0.984 | 2.63 | 3.54 | 1 | 6.18 |
| I-1 | 0.21 | 1.59 | 32.1 | NA | 189 | 61 | 61.8 |
| I-2 | 3.38 | 1.09 | NA | NA | 162 | 66.8 | 30.9 |
| I-3 | 0.455 | 3.71 | 60.6 | NA | 46.9 | 186 | 344 |
| I-4 | <0.0254 | 2.20 | 16.6 | NA | 69.5 | 52.9 | 123 |
| I-5 | <0.0254 | 1.99 | 25.6 | NA | 483 | 95 | 214 |
| I-6 | 11.5 | 15.4 | 94.7 | NA | >500 | 245 | 293 |
| I-7 | <0.0254 | 1.34 | 31.5 | NA | >500 | 99.1 | 189 |
| I-8 | 0.0306 | 2.95 | 89.1 | NA | >500 | >500 | >500 |
| I-9 | <0.0254 | 0.88 | 15 | NA | 91.1 | 39.5 | 22.5 |
| I-10 | 0.143 | 3.63 | 111 | 451 | >500 | 292 | 435 |
| I-11 | 1.19 | 1.58 | 29.8 | NA | >500 | 98.3 | 116 |
| I-12 | 1.25 | 1.26 | 15 | NA | 301 | 66 | 67 |
| I-13 | 3.48 | 3.17 | 93.2 | NA | 104 | 109 | 104 |
| I-14 | 11.9 | 8.34 | 70.9 | NA | 436 | 46.1 | 23.9 |
| I-15 | 26.3 | 19.3 | 118 | NA | >500 | 267 | 330 |
| II-1 | 0.214 | 2.91 | 75.1 | NA | NA | NA | NA |
| III-1 | 135 | 47.2 | NA | NA | NA | NA | NA |
| IV-1 | 59.9 | 32.1 | 137 | NA | NA | NA | NA |
| V-1 | 0.175 | 1.44 | 21.3 | NA | NA | 221 | 298 |
| VI-1 | 1.55 | 1.50 | 46 | 397 | 89.1 | 245 | 388 |

The above is a further detailed description of the present disclosure in connection with the specific preferred embodiments, and the specific embodiments of the present disclosure are not limited to the description. It will be apparent to those skilled in the art that the present disclosure may be practiced by making various simple deduction and replacement, without departing from the spirit and scope of the invention.

1. A compound of formula (I), or a pharmaceutically acceptable salt, enantiomer, diastereomer, racemate, or mixture thereof: 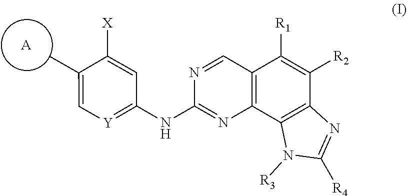
wherein:
Y is N, or CR₆;
wherein R₆ is H, —ORₐ, —SRₐ, —NR_{b}R_{c}, —C(O)Rₐ, —C(O)ORₐ, —C(O)NR_{b}R_{c}, —O—C(O)Rₐ, —O—C(O)ORₐ, —O—C(O)NR_{b}R_{c}, —N(R_{b})—C(O)Rₐ, —N(R_{b})—C(O)ORₐ, or —N(R_{b})—C(O)NR_{b}R_{c}, C₁₋₆ alkyl, or C₁₋₆ haloalkyl;
X is —ORₐ, —SRₐ, —NR_{b}R_{c}, —C(O)Rₐ, —C(O)ORₐ, —C(O)NR_{b}R_{c}, —O—C(O)Rₐ, —O—C(O)ORₐ, —O—C(O)NR_{b}R_{c}, —N(R_{b})—C(O)Rₐ, —N(R_{b})—C(O)ORₐ, or —N(R_{b})—C(O)NR_{b}R_{c};
wherein Rₐ is H, C₁₋₆ alkyl, C₁₋₆ haloalkyl, C₂₋₆ alkenyl, C₂₋₆ alkynyl, -L-C₃₋₇ cycloalkyl, -L-3- to 7-membered heterocyclyl, -L-C₆₋₁₀ aryl, or -L-5- to 10-membered heteroaryl;
R_{b} is H, C₁₋₆ alkyl, C₁₋₆ haloalkyl, C₂₋₆ alkenyl, C₂₋₆ alkynyl, -L-C₃₋₇ cycloalkyl, -L-3- to 7-membered heterocyclyl, -L-C₆₋₁₀ aryl, or -L-5- to 10-membered heteroaryl;
R_{c} is H, C₁₋₆ alkyl, C₁₋₆ haloalkyl, C₂₋₆ alkenyl, C₂₋₆ alkynyl, -L-C₃₋₇ cycloalkyl, -L-3- to 7-membered heterocyclyl, -L-C₆₋₁₀ aryl, or -L-5- to 10-membered heteroaryl;
or R_{b}, R_{c} and N atom to which they are attached to form a 3- to 7-membered heterocyclyl, or 5- to 10-membered heteroaryl;
wherein L is selected from a chemical bond, —C₁₋₆ alkylene-, —C₂₋₆ alkenylene-, or —C₂₋₆ alkynylene-;
ring A is -L′-3- to 11-membered heterocyclyl, which is optionally substituted with 1, 2, 3, 4, 5, 6, 7, or 8 R₅ groups;
wherein L′ is selected from a chemical bond, —O—, —S—, —NH—, —O—CH₂—, —CH₂—O—, —NH—CH₂—, or —CH₂—NH—;
R₅ is H, halo, oxo, —OR, —SR—, —NR′R″, C₁₋₆ alkyl, or C₁₋₆ haloalkyl; or, two of R₅s may link together to form a —C₁₋₄ alkylene-, —C₂₋₄ alkenylene- or —C₂₋₄ alkynylene-;
R, R′ and R″ is each independently H, C₁₋₆ alkyl, C₁₋₆ haloalkyl, C₂₋₆ alkenyl, or C₂₋₆ alkynyl; or R′, R″, and N atom to which they are attached to form a 3- to 7-membered heterocyclyl, or 5- to 10-membered heteroaryl;
R₁ is H, halogen, —CN, —ORₐ, —SRₐ, —NR_{b}R_{c}, C₁₋₆ alkyl, or C₁₋₆ haloalkyl;
R₂ is H, halogen, —CN, —ORₐ, —SRₐ, —NR_{b}R_{c}, C₁₋₆ alkyl, or C₁₋₆ haloalkyl;
R₃ is C₁₋₆ alkyl, C₁₋₆ haloalkyl, C₃₋₇ cycloalkyl, 3- to 7-membered heterocyclyl, C₆₋₁₀ aryl, or 5- to 10-membered heteroaryl; and
R₄ is H, halogen, —CN, —ORₐ, —SRₐ, —NR_{b}R_{c}, C₁₋₆ alkyl, or C₁₋₆ haloalkyl.

2. The compound of formula (I), or the pharmaceutically acceptable salt, enantiomer, diastereomer, racemate, or mixture thereof according to claim 1, wherein, Y is N.

3. The compound of formula (I), or the pharmaceutically acceptable salt, enantiomer, diastereomer, racemate, or mixture thereof according to claim 1, wherein,
X is —ORₐ, —SRₐ, —NR_{b}R_{c}, —C(O)Rₐ, —C(O)ORₐ, —C(O)NR_{b}R_{c}, —O—C(O)Rₐ, or —N(R_{b})—C(O)Rₐ;
preferably, X is —ORₐ, —SRₐ, or —NRₐR_{c};
preferably, X is —ORₐ;
preferably, X is —NR_{b}R_{c}.

4. The compound of formula (I), or the pharmaceutically acceptable salt, enantiomer, diastereomer, racemate, or mixture thereof according to claim 3, wherein,
Rₐ is H, C₁₋₆ alkyl, C₁₋₆ haloalkyl, C₂₋₆ alkenyl, C₂₋₆ alkynyl, -L-C₃₋₇ cycloalkyl, or -L-C₆₋₁₀ aryl; and
wherein L is selected from a chemical bond, or —C₁₋₆ alkylene-;
preferably,
Rₐ is C₁₋₆ alkyl, C₁₋₆ haloalkyl, C₂₋₆ alkenyl, C₂₋₆ alkynyl, or -L-C₃₋₇ cycloalkyl; and
wherein L is selected from a chemical bond, or —C₁₋₆ alkylene-.

5. The compound of formula (I), or the pharmaceutically acceptable salt, enantiomer, diastereomer, racemate, or mixture thereof according to claim 3, wherein,
R_{b} is H, C₁₋₆ alkyl, C₁₋₆ haloalkyl, C₂₋₆ alkenyl, C₂₋₆ alkynyl, -L-C₃₋₇ cycloalkyl, or -L-C₆₋₁₀ aryl;
R_{c} is H, C₁₋₆ alkyl, C₁₋₆ haloalkyl, C₂₋₆ alkenyl, C₂₋₆ alkynyl, -L-C₃₋₇ cycloalkyl, or -L-C₆₋₁₀ aryl;
or R_{b}, R_{c} and N atom to which they are attached to form a 3- to 7-membered heterocyclyl, or 5- to 10-membered heteroaryl; and
wherein L is selected from a chemical bond, or —C₁₋₆ alkylene-;
preferably,
R_{b} is H, C₁₋₆ alkyl, C₁₋₆ haloalkyl, C₂₋₆ alkenyl, C₂₋₆ alkynyl, or -L-C₃₋₇ cycloalkyl;
R_{c} is H, C₁₋₆ alkyl, C₁₋₆ haloalkyl, C₂₋₆ alkenyl, C₂₋₆ alkynyl, or -L-C₃₋₇ cycloalkyl;
or R_{b}, R_{c} and N atom to which they are attached to form a 3- to 7-membered heterocyclyl; and
wherein L is selected from a chemical bond, or —C₁₋₆ alkylene-;
preferably,
R_{b} is H, C₁₋₆ alkyl, or C₁₋₆ haloalkyl;
R_{c} is H, C₁₋₆ alkyl, or C₁₋₆ haloalkyl;
or R_{b}, R_{c} and N atom to which they are attached to form a 4- to 6-membered heterocyclyl.

6. The compound of formula (I), or the pharmaceutically acceptable salt, enantiomer, diastereomer, racemate, or mixture thereof according to claim 1, wherein,
ring A is -L′-3- to 7-membered heterocyclyl, which is optionally substituted with 1, 2, 3, 4, 5, 6, 7, or 8 R₅ groups;
wherein L′ is selected from a chemical bond, —O—CH₂—, —CH₂—O—, —NH—CH₂—, or —CH₂—NH—;
R₅ is H, halo, oxo, —OR, —SR—, —NR′R″, C₁₋₆ alkyl, or C₁₋₆ haloalkyl; or, two of R₅s may link together to form a —C₁₋₄ alkylene-, —C₂₋₄ alkenylene- or —C₂₋₄ alkynylene-; and
R, R′ and R″ is each independently H, C₁₋₆ alkyl, or C₁₋₆ haloalkyl; or R′, R″, and N atom to which they are attached to form a 3- to 7-membered heterocyclyl, or 5- to 10-membered heteroaryl;
preferably,
ring A is -L′-4- to 6-membered heterocyclyl, which is optionally substituted with 1, 2, 3, 4, 5, 6, 7, or 8 R₅ groups;
wherein L′ is selected from a chemical bond, —O—CH₂—, —CH₂—O—, —NH—CH₂—, or —CH₂—NH—;
R₅ is H, oxo, —OR, —NR′R″, C₁₋₆ alkyl, or C₁₋₆ haloalkyl; or, two of R₅s may link together to form a —C₁₋₄ alkylene-; and
R, R′ and R″ is each independently H, C₁₋₆ alkyl, or C₁₋₆ haloalkyl; or R′, R″, and N atom to which they are attached to form a 4- to 6-membered heterocyclyl;
preferably,
ring A is -L′-4- to 6-membered heterocyclyl, which is optionally substituted with 1, 2, 3, 4, 5, 6, 7, or 8 R₅ groups;
wherein L′ is selected from a chemical bond, —O—CH₂—, —CH₂—O—, —NH—CH₂—, or —CH₂—NH—;
R₅ is H, oxo, —OR, —NR′R″, or C₁₋₆ alkyl; or, two of R₅s may link together to form a —C₁₋₄ alkylene-; and
R, R′ and R″ is each independently H, or C₁₋₆ alkyl; or R′, R″, and N atom to which they are attached to form a 4- to 6-membered heterocyclyl;
preferably,
ring A is 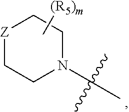
wherein Z is O, S, or NR₅;
R₅ is H, C₁₋₆ alkyl, or C₁₋₆ haloalkyl; or, two of R₅s may link together to form a —C₁₋₄ alkylene-, —C₂₋₄ alkenylene- or —C₂₋₄ alkynylene-;
m=1, 2, 3, 4, 5, 6, 7, or 8;
preferably,
ring A is 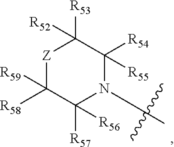
wherein Z: O, or NR₅₁;
R₅₁ to R₅₉ is H, or C₁₋₆ alkyl; or, two of R₅₁ to R₅₉ may link together to form a —C₁₋₄ alkylene-;
preferably,
ring A is 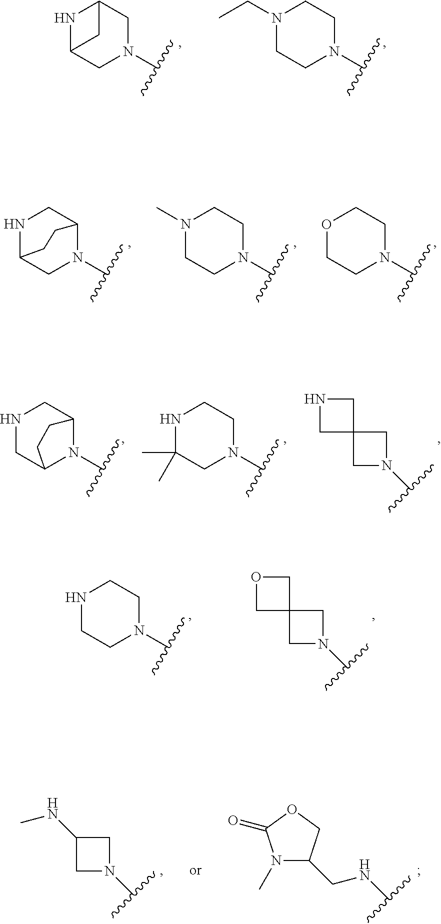
preferably,
ring A is

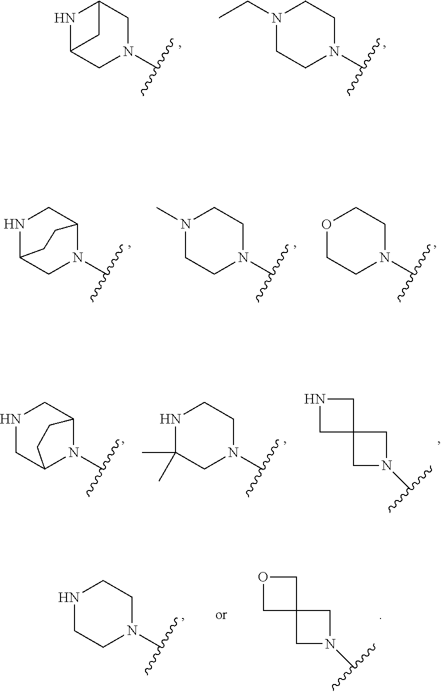

7. The compound of formula (I), or the pharmaceutically acceptable salt, enantiomer, diastereomer, racemate, or mixture thereof according to claim 1, wherein,
R₁ is H, C₁₋₆ alkyl, or C₁₋₆ haloalkyl;
preferably, R₁ is H.

8. The compound of formula (I), or the pharmaceutically acceptable salt, enantiomer, diastereomer, racemate, or mixture thereof according to claim 1, wherein,
R₂ is H, C₁₋₆ alkyl, or C₁₋₆ haloalkyl;
preferably, R₂ is H.

9. The compound of formula (I), or the pharmaceutically acceptable salt, enantiomer, diastereomer, racemate, or mixture thereof according to claim 1, wherein,
R₃ is C₁₋₆ alkyl, C₁₋₆ haloalkyl, C₃₋₇ cycloalkyl, or 3- to 7-membered heterocyclyl;
preferably, R₃ is C₁₋₆ alkyl, or C₁₋₆ haloalkyl;
preferably, R₃ is C₁₋₆ alkyl;
preferably, R₃ is Et or iPr.

10. The compound of formula (I), or the pharmaceutically acceptable salt, enantiomer, diastereomer, racemate, or mixture thereof according to claim 1, wherein,
R₄ is H, C₁₋₆ alkyl, or C₁₋₆ haloalkyl.
preferably, R₄ is H.

11. The compound of formula (I), or the pharmaceutically acceptable salt, enantiomer, diastereomer, racemate, or mixture thereof according to claim 1, wherein:
Y is N, or CR₆;
wherein R₆ is H, C₁₋₆ alkyl, or C₁₋₆ haloalkyl;
X is —ORₐ, —SRₐ, or —NR_{b}R_{c};
wherein Rₐ is H, C₁₋₆ alkyl, C₁₋₆ haloalkyl, C₂₋₆ alkenyl, C₂₋₆ alkynyl, -L-C₃₋₇ cycloalkyl, -L-3- to 7-membered heterocyclyl, -L-C₆₋₁₀ aryl, or -L-5- to 10-membered heteroaryl;
R_{b} is H, C₁₋₆ alkyl, or C₁₋₆ haloalkyl;
R_{c} is H, C₁₋₆ alkyl, or C₁₋₆ haloalkyl;
or R_{b}, R_{c} and N atom to which they are attached to form a 4- to 6-membered heterocyclyl, or 5- to 10-membered heteroaryl;
wherein L is selected from a chemical bond, —C₁₋₆ alkylene-, —C₂₋₆ alkenylene-, or —C₂₋₆ alkynylene-;
ring A is -L′-3- to 7-membered heterocyclyl, which is optionally substituted with 1, 2, 3, 4, 5, 6, 7, or 8 R₅ groups;
wherein L′ is selected from a chemical bond, —O—, —S—, —NH—, —O—CH₂—, —CH₂—O—, —NH—CH₂—, or —CH₂—NH—;
R₅ is H, halo, oxo, C₁₋₆ alkyl, C₁₋₆ haloalkyl, —OR, —SR—, or —NR′R″; or, two of R₅s may link together to form a —C₁₋₄ alkylene-, —C₂₋₄ alkenylene- or —C₂₋₄ alkynylene-;
R, R′ and R″ is each independently H, C₁₋₆ alkyl, or C₁₋₆ haloalkyl; or R′, R″, and N atom to which they are attached to form a 3- to 7-membered heterocyclyl, or 5- to 10-membered heteroaryl;
R₁ is H, C₁₋₆ alkyl, or C₁₋₆ haloalkyl;
R₂ is H, C₁₋₆ alkyl, or C₁₋₆ haloalkyl;
R₃ is C₁₋₆ alkyl, or C₁₋₆ haloalkyl; and
R₄ is H, C₁₋₆ alkyl, or C₁₋₆ haloalkyl.

12. The compound of formula (I), or the pharmaceutically acceptable salt, enantiomer, diastereomer, racemate, or mixture thereof according to claim 11, wherein:
Y is N, or CR₆;
wherein R₆ is H, or C₁₋₆ alkyl;
X is —ORₐ, or —NR_{b}R_{c};
wherein Rₐ is C₁₋₆ alkyl, C₁₋₆ haloalkyl, C₂₋₆ alkenyl, C₂₋₆ alkynyl, -L-C₃₋₇ cycloalkyl, or -L-C₆₋₁₀ aryl;
R_{b} is H, or C₁₋₆ alkyl;
R_{c} is H, or C₁₋₆ alkyl;
or R_{b}, R_{c} and N atom to which they are attached to form a 4- to 6-membered heterocyclyl;
wherein L is selected from a chemical bond, or —C₁₋₆ alkylene-;
ring A is -L′-4- to 6-membered heterocyclyl, which is optionally substituted with 1, 2, 3, 4, 5, 6, 7, or 8 R₅ groups;
wherein L′ is selected from a chemical bond, —O—CH₂—, —CH₂—O—, —NH—CH₂—, or —CH₂—NH—;
R₅ is H, oxo, C₁₋₆ alkyl, —OR, or —NR′R″; or, two of R₅s may link together to form a —C₁₋₄ alkylene-;
R, R′ and R″ is each independently H, or C₁₋₆ alkyl; or R′, R″, and N atom to which they are attached to form a 4- to 6-membered heterocyclyl, or 5- to 6-membered heteroaryl;
R₁ is H, or C₁₋₆ alkyl;
R₂ is H, or C₁₋₆ alkyl;
R₃ is C₁₋₆ alkyl; and
R₄ is H, or C₁₋₆ alkyl.

13. The compound of formula (I), or the pharmaceutically acceptable salt, enantiomer, diastereomer, racemate, or mixture thereof according to claim 11, wherein:
Y is N, or CH;
X is —ORₐ, or —NR_{b}R_{c};
wherein Rₐ is Me, Et, iPr, 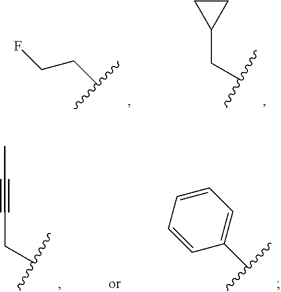
R_{b} is H, Me;
R_{c} is H, Me;
or R_{b}, R_{c} and N atom to which they are attached to form a 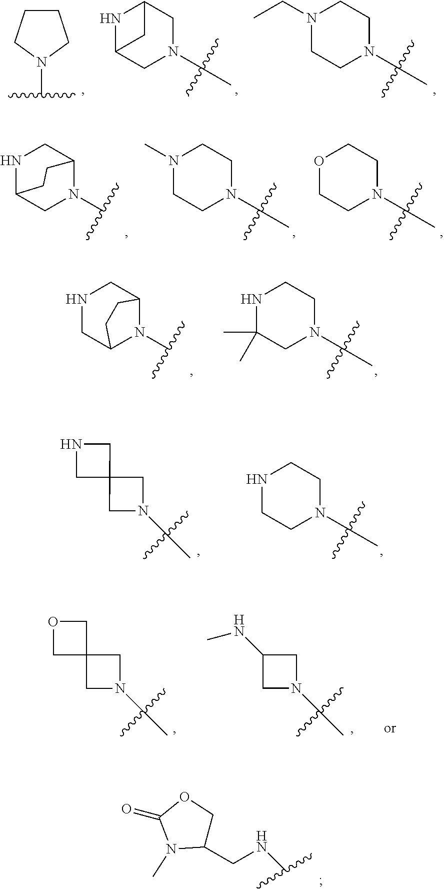
ring A is 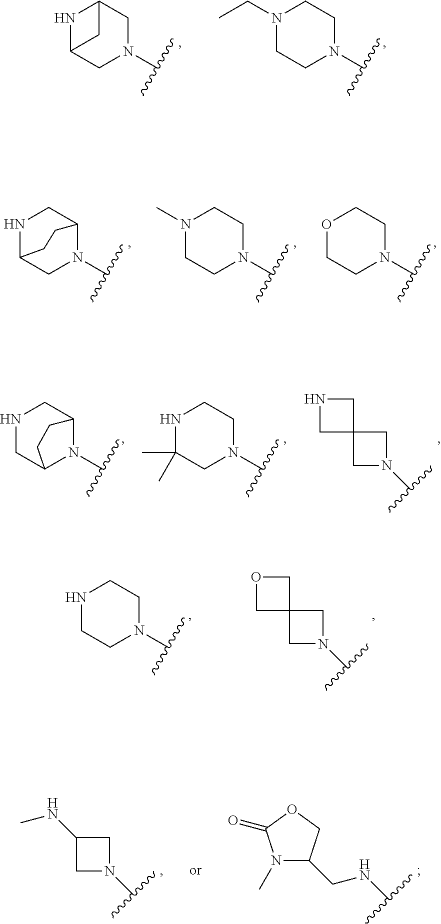
R₁ is H, or Me;
R₂ is H, or Me;
R₃ is Me, Et, or iPr; and
R₄ is H, or Me.

14. The compound of formula (I), or the pharmaceutically acceptable salt, enantiomer, diastereomer, racemate, or mixture thereof according to claim 1, which is a compound of formula (II): 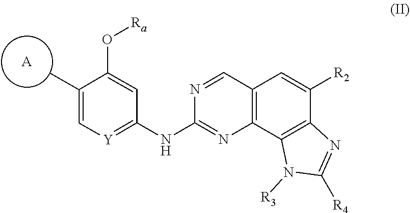
Y is N, or CH;
ring A is -L′-3- to 7-membered heterocyclyl, which is optionally substituted with 1, 2, 3, 4, 5, 6, 7, or 8 R₅ groups;
wherein L′ is selected from a chemical bond, —O—, —S—, —NH—, —O—CH₂—, —CH₂—O—, —NH—CH₂—, or —CH₂—NH—;
R₅ is H, halo, oxo, C₁₋₆ alkyl, C₁₋₆ haloalkyl, —OR, —SR—, or —NR′R″; or, two of R₅s may link together to form a —C₁₋₄ alkylene-, —C₂₋₄ alkenylene- or —C₂₋₄ alkynylene-;
R, R′ and R″ is each independently H, C₁₋₆ alkyl, or C₁₋₆ haloalkyl; or R′, R″, and N atom to which they are attached to form a 4- to 6-membered heterocyclyl, or 5- to 10-membered heteroaryl;
Rₐ is H, C₁₋₆ alkyl, C₁₋₆ haloalkyl, C₂₋₆ alkenyl, C₂₋₆ alkynyl, -L-C₃₋₇ cycloalkyl, -L-3- to 7-membered heterocyclyl, -L-C₆₋₁₀ aryl, or -L-5- to 10-membered heteroaryl;
wherein L is selected from a chemical bond, —C₁₋₆ alkylene-, —C₂₋₆ alkenylene-, or —C₂₋₆ alkynylene-;
R₂ is H, C₁₋₆ alkyl, or C₁₋₆ haloalkyl;
R₃ is Et, or iPr; and
R₄ is H, C₁₋₆ alkyl, or C₁₋₆ haloalkyl,
alternatively,
Y is N, or CH;
ring A is -L′-4- to 6-membered heterocyclyl, which is optionally substituted with 1, 2, 3, 4, 5, 6, 7, or 8 R₅ groups;
L′ is selected from a chemical bond, —O—CH₂—, —CH₂—O—, —NH—CH₂—, or —CH₂—NH—;
R₅ is H, oxo, C₁₋₆ alkyl, —OR, or —NR′R″; or, two of R₅s may link together to form a —C₁₋₄ alkylene-;
R, R′ and R″ is each independently H, or C₁₋₆ alkyl; or R′, R″, and N atom to which they are attached to form a 4- to 6-membered heterocyclyl, or 5- to 6-membered heteroaryl;
Rₐ is C₁₋₆ alkyl, C₁₋₆ haloalkyl, C₂₋₆ alkenyl, C₂₋₆ alkynyl, -L-C₃₋₇ cycloalkyl, or -L-C₆₋₁₀ aryl;
wherein L is selected from a chemical bond, or —C₁₋₆ alkylene-,
R₂ is H, or C₁₋₆ alkyl;
R₃ is Et, or iPr; and
R₄ is H, or C₁₋₆ alkyl;
alternatively,
Y is N, or CH;
ring A is 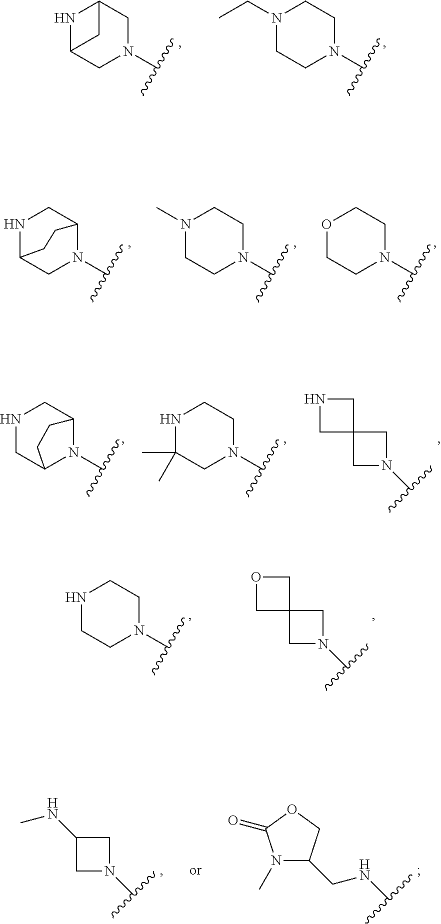
Rₐ is Me, Et, iPr, 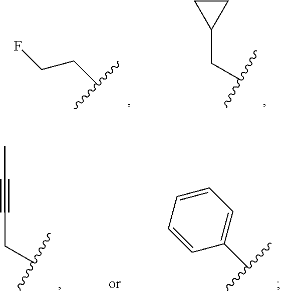
R₂ is H, or Me;
R₃ is Et, or iPr; and
R₄ is H, or Me.

15-16. (canceled)

17. The compound of formula (I), or the pharmaceutically acceptable salt, enantiomer, diastereomer, racemate, or mixture thereof according to claim 1, which is a compound of formula (II): 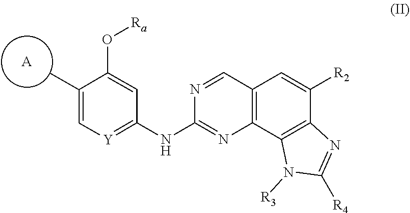
wherein:
Y is N, or CH;
ring A is 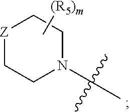
wherein Z is O, S, or NR₅;
R₅ is H, C₁₋₆ alkyl, or C₁₋₆ haloalkyl; or, two of R₅s may link together to form a —C₁₋₄ alkylene-, —C₂₋₄ alkenylene- or —C₂₋₄ alkynylene-;
m=1, 2, 3, 4, 5, 6, 7, or 8;
Rₐ is H, C₁₋₆ alkyl, C₁₋₆ haloalkyl, C₂₋₆ alkenyl, C₂₋₆ alkynyl, -L-C₃₋₇ cycloalkyl, or -L-3- to 7-membered heterocyclyl;
wherein L is selected from a chemical bond, —C₁₋₆ alkylene-, —C₂₋₆ alkenylene-, or —C₂₋₆ alkynylene-;
R₂ is H, C₁₋₆ alkyl, or C₁₋₆ haloalkyl;
R₃ is Et, or iPr; and
R₄ is H, C₁₋₆ alkyl, or C₁₋₆ haloalkyl;
alternatively,
Y is N, or CH;
ring A is 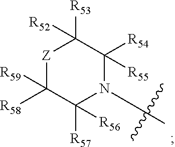
wherein Z is O, or NR₅₁;
R₅₁ to R₅₉ is each independently H, or C₁₋₆ alkyl; or, two of R₅₁ to R₅₉ may link together to form a —C₁₋₄ alkylene-;
Rₐ is C₁₋₆ alkyl, C₁₋₆ haloalkyl, C₂₋₆ alkenyl, C₂₋₆ alkynyl, or -L-C₃₋₇ cycloalkyl; wherein L is selected from a chemical bond, or —C₁₋₆ alkylene-;
R₂ is H, or C₁₋₆ alkyl;
R₃ is Et, or iPr; and
R₄ is H, or C₁₋₆ alkyl-,
alternatively,
Y is N, or CH;
ring A is 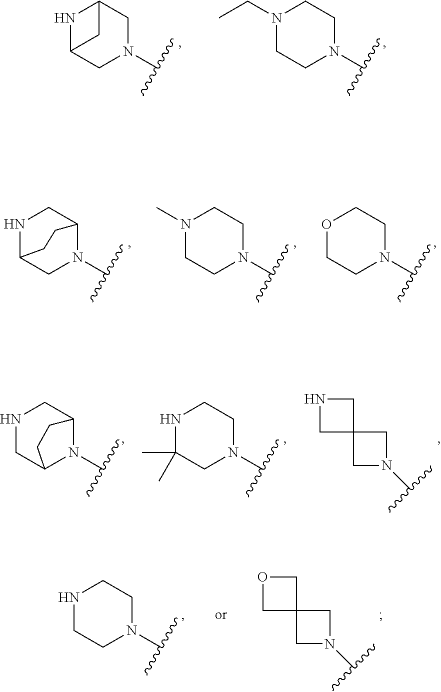
Rₐ is Me, Et, iPr, 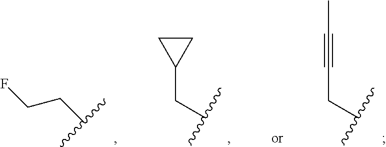
R₂ is H, or Me,
R₃ is Et, or iPr;
R₄ is H, or Me.

18-19. (canceled)

20. The compound, or the pharmaceutically acceptable salt, enantiomer, diastereomer, racemate, or mixture thereof according to claim 1, wherein the said compound is selected from:

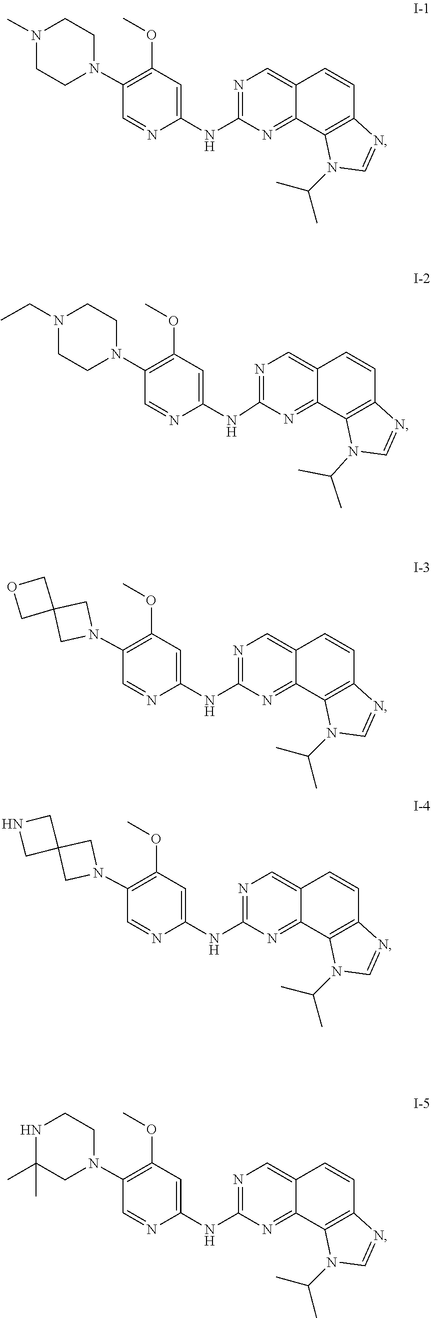 [SMILES: COc1cc(Nc2ncc3ccc4ncn(C(C)C)c4c3n2)ncc1N1CCN(C)CC1 ; CCN1CCN(c2cnc(Nc3ncc4ccc5ncn(C(C)C)c5c4n3)cc2OC)CC1 ; COc1cc(Nc2ncc3ccc4ncn(C(C)C)c4c3n2)ncc1N1CC2(COC2)C1 ; COc1cc(Nc2ncc3ccc4ncn(C(C)C)c4c3n2)ncc1N1CC2(CNC2)C1 ; COc1cc(Nc2ncc3ccc4ncn(C(C)C)c4c3n2)ncc1N1CCNC(C)(C)C1]

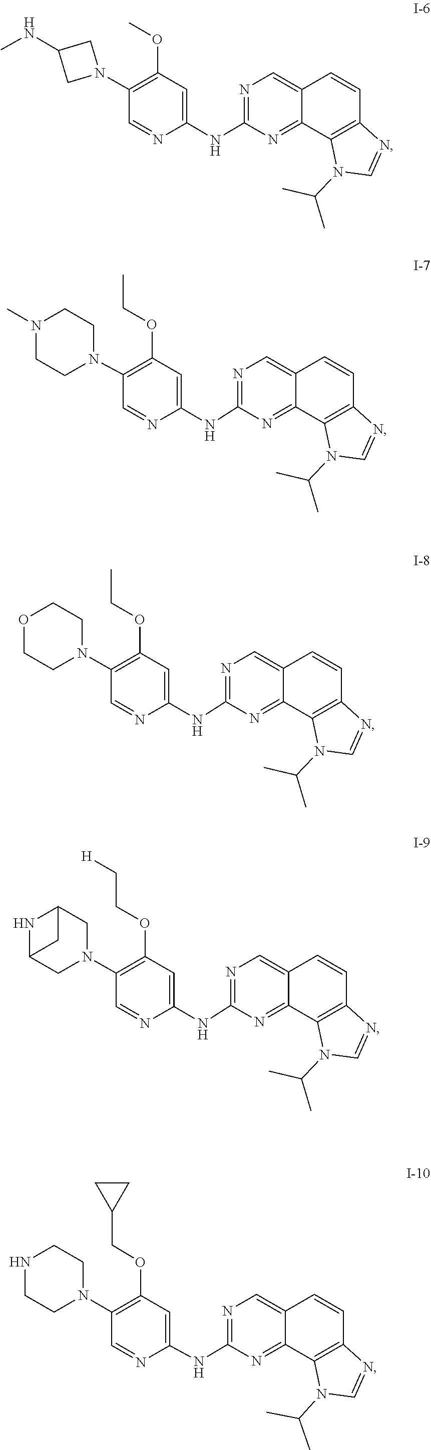 [SMILES: CNC1CN(c2cnc(Nc3ncc4ccc5ncn(C(C)C)c5c4n3)cc2OC)C1 ; CCOc1cc(Nc2ncc3ccc4ncn(C(C)C)c4c3n2)ncc1N1CCN(C)CC1 ; CCOc1cc(Nc2ncc3ccc4ncn(C(C)C)c4c3n2)ncc1N1CCOCC1 ; CCOc1cc(Nc2ncc3ccc4ncn(C(C)C)c4c3n2)ncc1N1CC2CC(C1)N2 ; CC(C)n1cnc2ccc3cnc(Nc4cc(OCC5CC5)c(N5CCNCC5)cn4)nc3c21]

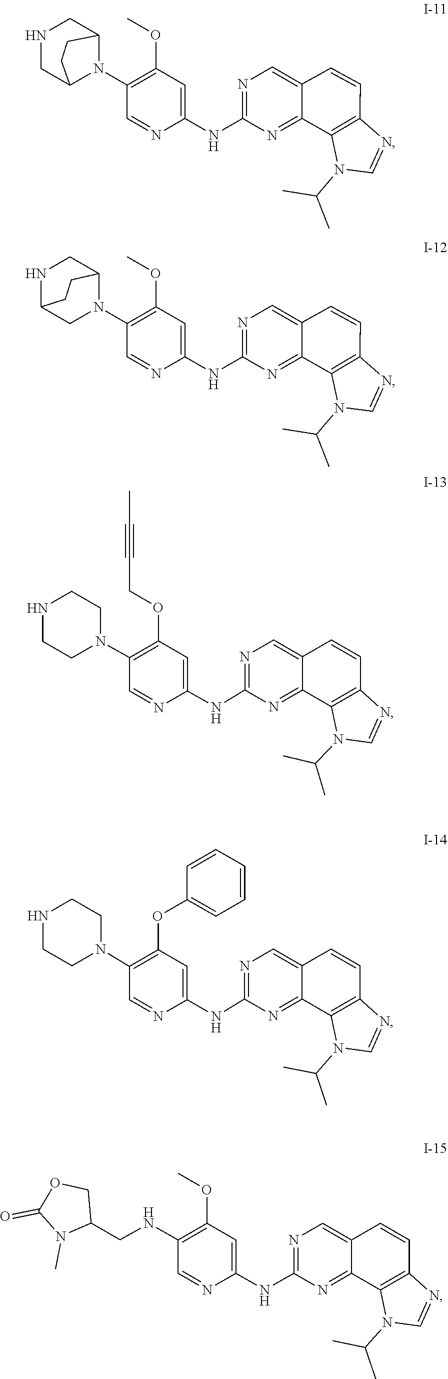

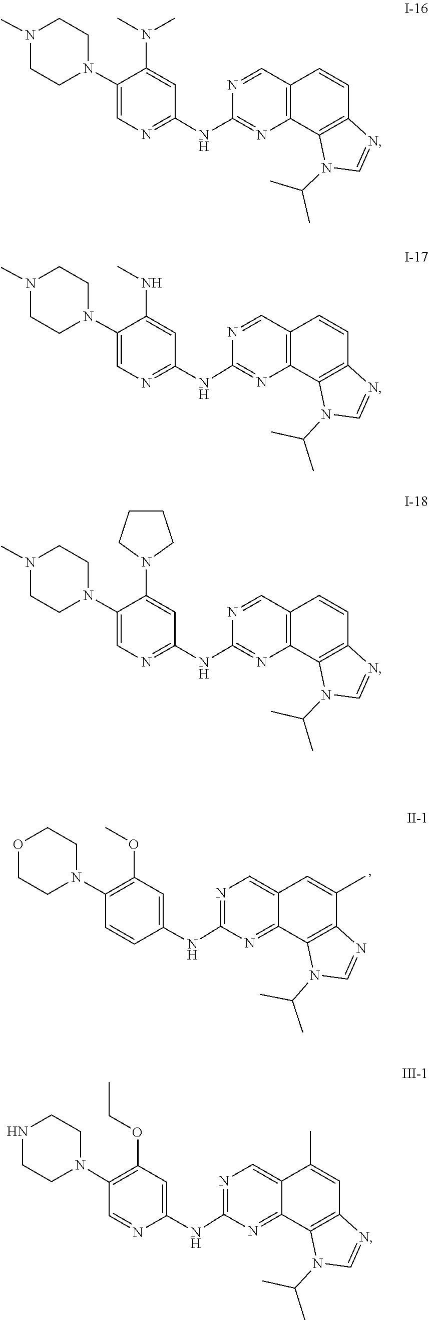 [SMILES: CC(C)n1cnc2ccc3cnc(Nc4cc(N(C)C)c(N5CCN(C)CC5)cn4)nc3c21 ; CNc1cc(Nc2ncc3ccc4ncn(C(C)C)c4c3n2)ncc1N1CCN(C)CC1 ; CC(C)n1cnc2ccc3cnc(Nc4cc(N5CCCC5)c(N5CCN(C)CC5)cn4)nc3c21 ; COc1cc(Nc2ncc3cc(C)c4ncn(C(C)C)c4c3n2)ccc1N1CCOCC1 ; CCOc1cc(Nc2ncc3c(C)cc4ncn(C(C)C)c4c3n2)ncc1N1CCNCC1]

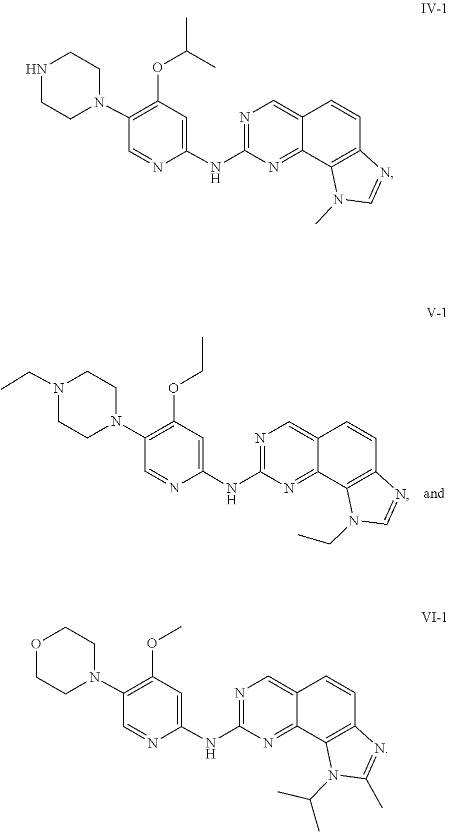 [SMILES: CC(C)Oc1cc(Nc2ncc3ccc4ncn(C)c4c3n2)ncc1N1CCNCC1 ; CCOc1cc(Nc2ncc3ccc4ncn(CC)c4c3n2)ncc1N1CCN(CC)CC1 ; COc1cc(Nc2ncc3ccc4nc(C)n(C(C)C)c4c3n2)ncc1N1CCOCC1]

21. A pharmaceutical composition, comprising:
the compound, or the pharmaceutically acceptable salt, enantiomer, diastereomer, racemate, or mixture thereof according to claim 1;
pharmaceutically acceptable excipient(s); and
optionally, one or more other therapeutic agents.

22. A kit, comprising:
a first container which contains the compound, or the pharmaceutically acceptable salt, enantiomer, diastereomer, racemate, or mixture thereof according to claim 1; and
optionally, a second container which contains one or more other therapeutic agents; and
optionally, a third container which contains pharmaceutically acceptable excipient(s) for diluting or suspending the said compound and/or other therapeutic agent(s).

23. (canceled)

24. A method of treating and/or preventing a FLT3 mediated disease in a subject, which comprises administering to the subject the compound, or the pharmaceutically acceptable salt, enantiomer, diastereomer, racemate, or mixture thereof according to claim 1.

25. (canceled)

26. The method of claim 24, wherein the said FLT3 mediated disease includes a cell proliferative disorder, including but not limited to, a leukemia, myeloma, myeloproliferative disease, mylodysplastic syndrome, idiopathic hypereosinophilic syndrome (HES), bladder cancer, breast cancer, cervical cancer, CNS cancer, colon cancer, esophageal cancer, head and neck cancer, liver cancer, lung cancer, nasopharyngeal cancer, neuroendocrine cancer, ovarian cancer, pancreatic cancer, prostate cancer, renal cancer, salivary gland cancer, small cell lung cancer, skin cancer, stomach cancer, testicular cancer, thyroid cancer, uterine cancer, and hematologic malignancy.
